(19) United States
(12) Patent Application Publication
(10) Pub. No.: US 2023/0410977 A1
(43) Pub. Date: Dec. 21, 2023

(54) METHODS AND MATERIALS FOR ASSESSING RADIATION EXPOSURE

(71) Applicant: NUtech Ventures, Lincoln, NE (US)

(72) Inventors: Alexandra Crook, Lincoln, NE (US); Aline De Lima Leite, Lincoln, NE (US); Thomas Payne, Lincoln, NE (US); Fatema Bhinderwala, Lincoln, NE (US); Robert Powers, Lincoln, NE (US)

(21) Appl. No.: 18/268,721

(22) PCT Filed: Dec. 22, 2021
(86) PCT No.: PCT/US2021/064896

§ 371 Date: Jun. 21, 2023

Related U.S. Application Data
(60) Provisional application No. 63/129,394, filed on Dec. 22, 2020.

(51) Int. Cl. G16H 20/40; A61N 5/10; G01N 33/574; G01N 33/68; G16H 10/40

(52) U.S. Cl. CPC G16H 20/40; A61N 5/103; G01N 33/57488; G01N 33/6848; G16H 10/40

(57) ABSTRACT

This document relates to methods and materials for assessing radiation exposure. For example, methods and materials that can be used to determine whether a mammal (e.g., a human) has been exposed to radiation and/or whether a mammal (e.g., a human) is at risk of developing one or more radiation injuries (e.g., cutaneous radiation injury (CRI) and/or acute radiation syndrome (ARS)) following radiation exposure (e.g., radiation therapy) are provided.

### CROSS-REFERENCE TO RELATED APPLICATIONS

This application claims the benefit of U.S. Patent Application Ser. No. 63/129,394, filed on Dec. 22, 2020. The disclosure of the prior application is considered part of (and is incorporated by reference in) the disclosure of this application.

### STATEMENT REGARDING FEDERAL FUNDING

This invention was made with government support under 17-FU910 awarded by the United States Air Force, National Strategic Research Institute and under P20 GM113126 awarded by the National Institutes of Health. The government has certain rights in the invention.

### BACKGROUND

### 1. Technical Field

This document relates to methods and materials for assessing radiation exposure. For example, methods and materials provided herein can be used to determine if a mammal (e.g., a human) has been exposed to radiation and/or if a mammal (e.g., a human) is at risk of developing one or more radiation injuries (e.g., cutaneous radiation injury (CRI) and/or acute radiation syndrome (ARS)) following radiation exposure (e.g., radiation therapy). In some cases, this document provides methods and materials for using one or more radiation counter-measures to treat a mammal exposed to radiation and/or at risk of developing one or more radiation injuries (e.g., CRI and/or ARS) following radiation exposure (e.g., radiation therapy).

### 2. Background Information

Exposure to γ-radiation has both short- and long-term effects on human health and survivability (Gonzalez et al., 2009 Health Physics, 97:6-49). Injuries incurred as a result of radiation exposure fall into three categories, ARS, delayed or late arising pathologies, and chronic illnesses (DiCarlo et al., 2011 Disaster Med Public Health Prep, 5(Suppl 1):S32-44). Tools for diagnosing radiation injury and effective treatment strategies for radiation injuries are needed (Pellmar et al., 2005 Radiation Research, 163(1):115-123).

### SUMMARY

This document provides methods and materials for assessing radiation exposure. For example, methods and materials provided herein can be used to determine whether a mammal (e.g., a human) has been exposed to radiation and/or whether a mammal (e.g., a human) is at risk of developing one or more radiation injuries (e.g., CRI and/or ARS) following radiation exposure (e.g., radiation therapy administered as a part of a cancer treatment). In some cases, this document provides methods and materials for using one or more radiation counter-measures to treat a mammal exposed to radiation and/or at risk of developing one or more radiation injuries (e.g., CRI and/or ARS) following radiation exposure (e.g., radiation therapy administered as a part of a cancer treatment). In some cases, this document provides methods and materials for monitoring a mammal (e.g., a human such as a human having cancer) for effectiveness of a treatment with one or more radiation counter measures (e.g., radioprotective agents, radio-mitigation agents, and/or radio-therapeutic agents) following radiation exposure (e.g., radiation therapy administered as a part of a cancer treatment). In some cases, this document provides methods and materials for identifying one or more candidate radiation counter measures (e.g., radioprotective agents, radio-mitigation agents, and/or radio-therapeutic agents) and/or assessing one or more candidate radiation counter measures (e.g., radioprotective agents, radio-mitigation agents, and/or radio-therapeutic agents) for effectiveness.

As demonstrated herein, the presence of an altered level (e.g., an increased level or a decreased level) of one or more metabolites and/or one or more metabolic pathways in a sample obtained from a mammal can be used to determine whether a mammal (e.g., a human) has been exposed to radiation. Also as demonstrated herein, the presence of an altered level (e.g., an increased level or a decreased level) of one or more metabolites and/or one or more metabolic pathways in a sample obtained from a mammal can be used to determine whether or not that mammal is likely to develop one or more radiation injuries (e.g., CRI and/or ARS) following radiation exposure (e.g., radiation therapy administered as a part of a cancer treatment). The metabolic biomarkers and the metabolic pathways described herein provide a unique and unrealized opportunity to identify a mammal as having been exposed to radiation and/or as being likely to develop one or more radiation injuries (e.g., CRI and/or ARS) following radiation exposure (e.g., radiation therapy administered as a part of a cancer treatment). For example, a mammal identified as being likely to develop one or more radiation injuries (e.g., CRI and/or ARS) following radiation exposure (e.g., radiation therapy administered as a part of a cancer treatment) as described herein (e.g., based, at least in part on the metabolic biomarkers and/or metabolic pathways described herein) can be administered one or more radiation counter-measures, such as amifostine, prior to radiation exposure to minimize or even prevent the development of any radiation injury.

Having the ability to identify a mammal that has been exposed to radiation (or is suspected of having been exposed to radiation) as being likely to develop one or more radiation injuries based, at least in part, on changes in one or more metabolic biomarkers and/or metabolic pathways as described herein provides a unique and unrealized opportunity to provide a personalized or precision approach to treatments.

In general, one aspect of this document features methods for determining whether or not a mammal is likely to develop a radiation injury following radiation exposure. The methods can include, or consist essentially of, (a) detecting a presence or absence of an altered level of at least five metabolites in a sample from a mammal having been exposed to radiation; (b) classifying the mammal as being likely to develop a radiation injury following radiation exposure if said presence of the altered level is detected; and (c) classifying the mammal as not being likely to develop a radiation injury following radiation exposure if the altered level is not detected. The altered level of the at least five metabolites can be an increased level, and the at least five metabolites can be 10,11-dihydro-12r-hydroxy-leukotriene E4, 12-oxo-20-trihydroxy-leukotriene B4, 2-amino-3-phosphonopropionic acid, 3-hydroxybutyrate, 5-hydroxyindoleacetic acid, 5-hydroxytryptophol glucuronide, 7-methylguanosine, acetyldigitoxin, alpha-CEHC, D-glucose, formamidopyrimidine nucleoside triphosphate, gamma glutamylglutamic acid, gamma-glutamylthreonine, ganglioside GA1 (d18:1/16:0), ganglioside GM2 (d18:1/18:1(11z)), L-beta-aspartyl-L-threonine, leucyl-valine, leukotriene F4, L-isoleucine, L-leucine, lysoPC(22:2(13z,16z)), N-a-acetyl-L-arginine, N-acetyltryptophan, N-gamma-L-glutamyl-D-alanine, PG(18:3(9Z,12Z,15Z)/20:3(5Z,8Z,11Z)), phenylalanylphenylalanine, phosphoribosyl-AMP, p-hydroxyphenylacetic acid, PS(14:0/14:1(9Z)), PS(20:4(5Z,8Z,11Z,14Z)/14:1(9Z)), pyruvate, tetrahexosylceramide (d18:1/16:0), trans-3-hydroxycotinine glucuronide, trimethylamine N-oxide, uridine, valine, vanylglycol, L-carnitine, tiglyl-CoA, 2,2-dimethylsuccinic acid, 3-methyl-2-oxovalerate, anserine, L-phenylalanine, L-tryptophan, N,N-Dimethylglycine, N6-acetyllysine, N-acetylysteine, propylene glycol, undecanoic acid, or xanthurenate. The altered level of the at least five metabolites can be a decreased level, and the at least five metabolites can be 1-methylhistidine, 2-aminomuconic acid semialdehyde, 2-methylguanosine, 2-oxoarginine, 2-pyrroloylglycine, 3-(3,5-diiodo-4-hydroxyphenyl)pyruvate, 3-dehydroxycarnitine, 3-hydroxyoctanoyl carnitine, 3-nitrotyrosine, 3′-O-methyladenosine, 4-guanidinobutanoic acid, 7-methylguanine, alanyl-serine, allantoic acid, argininic acid, arginyl-methionine, citric acid, creatine, cysteic acid, cysteinyl-cysteine, DL-2-aminooctanoic acid, dUMP, gamma-glutamyltyrosine, glutaminyl-lysine, glutarylcarnitine, glycerophosphocholine, glyceryl lactopalmitate, hippuric acid, IDP, L-acetylcarnitine, L-alanine, L-aspartyl-4-phosphate, L-carnitine, L-glutamine, L-histidine, L-isoleucine, L-phenylalanine, L-tyrosine, melatonin, N1-methyl-2-pyridone-5-carboxamide, N6,N6,N6-trimethyl-L-lysine, N-acetyl-L-methionine, nicotine imine, N-ribosylhistidine, 0-isobutyryl-L-carnitine, phenol sulphate, phenylpyruvic acid, picolinoylglycine, proline betaine, pyrogallol-2-O-glucuronide, riboflavin, serinyl-hydroxyproline, sphinganine 1-phosphate, trans-3-coumarate, tyrosyl-glutamate, urocanic acid, 1H-indole-3-carboxaldehyde, 2-hydroxyadenine, allodesmosine, arginyl-histidine, guanine, hypoxanthine, LysoPC(14:0), LysoPC(15:0), LysoPC(18:3(6Z,9Z,12Z)), LysoPE(0:0/18:1(11Z)), phosphoric acid, pyrrolidine, uridine, 1,9-dimethylurate, 2-(3-carboxy-3-aminopropyl)-L-histidine, adenosine diphosphate ribose, adenosine thiamine triphosphate, ADP, ATP, cholesterol glucuronide, D-galactose, D-glucose, GTP, malonyl-camitin, N-acetyl-D-glucosamine, NAD⁺, NADH, NADP⁺, N-decanoylglycine, phosphoribosyl-AMP, purine, or tiglylcarnitine. The detecting can include liquid chromatography-mass spectrometry (LC-MS), nuclear magnetic resonance (NMR), gas chromatography-mass spectrometry (GC-MS), capillary electrophoresis-mass spectrometry (CE-MS), Fourier transform infrared spectroscopy (FTIR), or combinations thereof. The mammal can be a human. The sample can be whole blood, serum, plasma, urine, cerebrospinal fluid (CSF), saliva, jejunum tissue, lung tissue, heart tissue, kidney tissue, skin tissue, bone marrow, gastrointestinal tract tissue, cardiovascular system tissue, central nervous system (CNS) tissue, hematopoietic cells, or a fecal sample. The radiation exposure can include radiation therapy. The mammal can have cancer. The radiation exposure can occur during an event selected from the group consisting of an industrial accident, terrorist attack, military action, commercial exposure, medical procedure, environmental exposure, and any combinations thereof. The radiation exposure ca include a radiation source selected from the group consisting of ionizing radiation, non-ionizing radiation, low energy particles, and high energy particles. The radiation injury can include a CRI and/or ARS.

In another aspect, this document features methods for determining whether or not a mammal is likely to develop a radiation injury following radiation exposure. The methods can include, or consist essentially of, (a) detecting a presence or absence of at least one enriched metabolic pathway in a sample from a mammal having been exposed to radiation; (b) classifying the mammal as being likely to develop a radiation injury following radiation exposure if stheaid presence of satheid enriched metabolic pathways is detected; and (c) classifying the mammal as not being likely to develop a radiation injury following radiation exposure if the enriched metabolic pathways is not detected. The at least one enriched metabolic pathway can be Warburg effect, arginine and proline metabolism, glutamate metabolism, pyruvate metabolism, fatty acid metabolism, propanoate metabolism, nicotinate and nicotinamide metabolism, gluconeogenesis, ammonia recycling, pyrimidine metabolism, urea cycle, selenoamino acid metabolism, mitochondrial beta-oxidation of long chain saturated fatty acids, mitochondrial beta-oxidation of short chain saturated fatty acids, cysteine metabolism, oxidation of branched chain fatty acids, glycolysis, purine metabolism, carnitine synthesis, glutathione metabolism, lactose synthesis, beta oxidation of very long chain fatty acids, or valine, leucine and isoleucine degradation. The mammal can be a human. The sample can be whole blood, serum, plasma, urine, CSF, saliva, jejunum tissue, lung tissue, heart tissue, kidney tissue, skin tissue, bone marrow, gastrointestinal tract tissue, cardiovascular system tissue, CNS tissue, hematopoietic cells, or a fecal sample. The radiation exposure can include radiation therapy. The mammal can have cancer. The radiation exposure can occur during an event selected from the group consisting of an industrial accident, terrorist attack, military action, commercial exposure, medical procedure, environmental exposure, and any combinations thereof. The radiation exposure ca include a radiation source selected from the group consisting of ionizing radiation, non-ionizing radiation, low energy particles, and high energy particles. The radiation injury can include a CRI and/or ARS.

In another aspect, this document features methods for treating a mammal having cancer. The methods can include, or consist essentially of, (a) detecting an absence of an altered level of at least five metabolites in a sample from a mammal having cancer; and (b) administering a radiation therapy to the mammal. The altered level of the at least five metabolites can be an increased level, and the at least five metabolites can be 10,11-dihydro-12r-hydroxy-leukotriene E4, 12-oxo-20-trihydroxy-leukotriene B4, 2-amino-3-phosphonopropionic acid, 3-hydroxybutyrate, 5-hydroxyindoleacetic acid, 5-hydroxytryptophol glucuronide, 7-methylguanosine, acetyldigitoxin, alpha-CEHC, D-glucose, formamidopyrimidine nucleoside triphosphate, gamma glutamylglutamic acid, gamma-glutamylthreonine, ganglioside GA1 (d18:1/16:0), ganglioside GM2 (d18:1/18:1(11z)), L-beta-aspartyl-L-threonine, leucyl-valine, leukotriene F4, L-isoleucine, L-leucine, lysoPC(22:2(13z,16z)), N-a-acetyl-L-arginine, N-acetyltryptophan, N-gamma-L-glutamyl-D-alanine, PG(18:3(9Z,12Z,15Z)/20:3(5Z,8Z,11Z)), phenylalanylphenylalanine, phosphoribosyl-AMP, p-hydroxyphenylacetic acid, PS(14:0/14:1(9Z)), PS(20:4(5Z,8Z,11Z,14Z)/14:1(9Z)), pyruvate, tetrahexosylceramide (d18:1/16:0), trans-3-hydroxycotinine glucuronide, trimethylamine N-oxide, uridine, valine, vanylglycol, L-carnitine, tiglyl-CoA, 2,2-dimethylsuccinic acid, 3-methyl-2-oxovalerate, anserine, L-phenylalanine, L-tryptophan, N,N-Dimethylglycine, N6-acetyllysine, N-acetylysteine, propylene glycol, undecanoic acid, or xanthurenate. The altered level of the at least five metabolites can be a decreased level, and threat least five metabolites can be of 1-methylhistidine, 2-aminomuconic acid semialdehyde, 2-methylguanosine, 2-oxoarginine, 2-pyrroloylglycine, 3-(3,5-diiodo-4-hydroxyphenyl)pyruvate, 3-dehydroxycarnitine, 3-hydroxyoctanoyl carnitine, 3-nitrotyrosine, 3′-O-methyladenosine, 4-guanidinobutanoic acid, 7-methylguanine, alanyl-serine, allantoic acid, argininic acid, arginyl-methionine, citric acid, creatine, cysteic acid, cysteinyl-cysteine, DL-2-aminooctanoic acid, dUMP, gamma-glutamyltyrosine, glutaminyl-lysine, glutarylcarnitine, glycerophosphocholine, glyceryl lactopalmitate, hippuric acid, IDP, L-acetylcarnitine, L-alanine, L-aspartyl-4-phosphate, L-carnitine, L-glutamine, L-histidine, L-isoleucine, L-phenylalanine, L-tyrosine, melatonin, N1-methyl-2-pyridone-5-carboxamide, N6,N6,N6-trimethyl-L-lysine, N-acetyl-L-methionine, nicotine imine, N-ribosylhistidine, O-isobutyryl-L-carnitine, phenol sulphate, phenylpyruvic acid, picolinoylglycine, proline betaine, pyrogallol-2-O-glucuronide, riboflavin, serinyl-hydroxyproline, sphinganine 1-phosphate, trans-3-coumarate, tyrosyl-glutamate, urocanic acid, 1H-indole-3-carboxaldehyde, 2-hydroxyadenine, allodesmosine, arginyl-histidine, guanine, hypoxanthine, LysoPC(14:0), LysoPC(15:0), LysoPC(18:3(6Z,9Z,12Z)), LysoPE(0:0/18:1(11Z)), phosphoric acid, pyrrolidine, uridine, 1,9-dimethylurate, 2-(3-carboxy-3-aminopropyl)-L-histidine, adenosine diphosphate ribose, adenosine thiamine triphosphate, ADP, ATP, cholesterol glucuronide, D-galactose, D-glucose, GTP, malonyl-carnitine, N-acetyl-D-glucosamine, NAD⁺, NADH, NADP⁺, N-decanoylglycine, phosphoribosyl-AMP, purine, or tiglylcarnitine. The detecting can include LC-MS, NMR, GC-MS, CE-MS, FTIR, or combinations thereof. The mammal can be a human. The sample can be whole blood, serum, plasma, urine, CSF, saliva, jejunum tissue, lung tissue, heart tissue, kidney tissue, skin tissue, bone marrow, gastrointestinal tract tissue, cardiovascular system tissue, CNS tissue, hematopoietic cells, or a fecal sample.

In another aspect, this document features methods for treating a mammal having cancer. The methods can include, or consist essentially of, (a) detecting an absence of at least one enriched metabolic pathway in a sample from a mammal having cancer; and (b) administering a radiation therapy to the mammal. The at least one enriched metabolic pathway can be Warburg effect, arginine and proline metabolism, glutamate metabolism, pyruvate metabolism, fatty acid metabolism, propanoate metabolism, nicotinate and nicotinamide metabolism, gluconeogenesis, ammonia recycling, pyrimidine metabolism, urea cycle, selenoamino acid metabolism, mitochondrial beta-oxidation of long chain saturated fatty acids, mitochondrial beta-oxidation of short chain saturated fatty acids, cysteine metabolism, oxidation of branched chain fatty acids, glycolysis, purine metabolism, carnitine synthesis, glutathione metabolism, lactose synthesis, beta oxidation of very long chain fatty acids, or valine, leucine and isoleucine degradation. The mammal can be a human. The sample can be whole blood, serum, plasma, urine, CSF, saliva, jejunum tissue, lung tissue, heart tissue, kidney tissue, skin tissue, bone marrow, gastrointestinal tract tissue, cardiovascular system tissue, CNS tissue, hematopoietic cells, or a fecal sample.

In another aspect, this document features methods for treating a mammal having cancer. The methods can include, or consist essentially of, (a) detecting a presence of an altered level of at least five metabolites in a sample from a mammal having cancer; and (b) administering a cancer treatment to the mammal, where the cancer treatment is not a radiation therapy. The altered level of the at least five metabolites can be an increased level, and the at least five metabolites can be 10,11-dihydro-12r-hydroxy-leukotriene E4, 12-oxo-20-trihydroxy-leukotriene B4, 2-amino-3-phosphonopropionic acid, 3-hydroxybutyrate, 5-hydroxyindoleacetic acid, 5-hydroxytryptophol glucuronide, 7-methylguanosine, acetyldigitoxin, alpha-CEHC, D-glucose, formamidopyrimidine nucleoside triphosphate, gamma glutamylglutamic acid, gamma-glutamylthreonine, ganglioside GA1 (d18:1/16:0), ganglioside GM2 (d18:1/18:1(11z)), L-beta-aspartyl-L-threonine, leucyl-valine, leukotriene F4, L-isoleucine, L-leucine, lysoPC(22:2(13z,16z)), N-a-acetyl-L-arginine, N-acetyltryptophan, N-gamma-L-glutamyl-D-alanine, PG(18:3(9Z,12Z,15Z)/20:3(5Z,8Z,11Z)), phenylalanylphenylalanine, phosphoribosyl-AMP, p-hydroxyphenylacetic acid, PS(14:0/14:1(9Z)), PS(20:4(5Z,8Z,11Z,14Z)/14:1(9Z)), pyruvate, tetrahexosylceramide (d18:1/16:0), trans-3-hydroxycotinine glucuronide, trimethylamine N-oxide, uridine, valine, vanylglycol, L-carnitine, tiglyl-CoA, 2,2-dimethylsuccinic acid, 3-methyl-2-oxovalerate, anserine, L-phenylalanine, L-tryptophan, N,N-Dimethylglycine, N6-acetyllysine, N-acetylysteine, propylene glycol, undecanoic acid, or xanthurenate. The altered level of the at least five metabolites can be a decreased level, and the at least five metabolites can be 1-methylhistidine, 2-aminomuconic acid semialdehyde, 2-methylguanosine, 2-oxoarginine, 2-pyrroloylglycine, 3-(3,5-diiodo-4-hydroxyphenyl)pyruvate, 3-dehydroxycarnitine, 3-hydroxyoctanoyl carnitine, 3-nitrotyrosine, 3′-O-methyladenosine, 4-guanidinobutanoic acid, 7-methylguanine, alanyl-serine, allantoic acid, argininic acid, arginyl-methionine, citric acid, creatine, cysteic acid, cysteinyl-cysteine, DL-2-aminooctanoic acid, dUMP, gamma-glutamyltyrosine, glutaminyl-lysine, glutarylcarnitine, glycerophosphocholine, glyceryl lactopalmitate, hippuric acid, IDP, L-acetylcarnitine, L-alanine, L-aspartyl-4-phosphate, L-carnitine, L-glutamine, L-histidine, L-isoleucine, L-phenylalanine, L-tyrosine, melatonin, N1-methyl-2-pyridone-5-carboxamide, N6,N6,N6-trimethyl-L-lysine, N-acetyl-L-methionine, nicotine imine, N-ribosylhistidine, O-isobutyryl-L-carnitine, phenol sulphate, phenylpyruvic acid, picolinoylglycine, proline betaine, pyrogallol-2-O-glucuronide, riboflavin, serinyl-hydroxyproline, sphinganine 1-phosphate, trans-3-coumarate, tyrosyl-glutamate, urocanic acid, 1H-indole-3-carboxaldehyde, 2-hydroxyadenine, allodesmosine, arginyl-histidine, guanine, hypoxanthine, LysoPC(14:0), LysoPC(15:0), LysoPC(18:3(6Z,9Z,12Z)), LysoPE(0:0/18:1(11Z)), phosphoric acid, pyrrolidine, uridine, 1,9-dimethylurate, 2-(3-carboxy-3-aminopropyl)-L-histidine, adenosine diphosphate ribose, adenosine thiamine triphosphate, ADP, ATP, cholesterol glucuronide, D-galactose, D-glucose, GTP, malonyl-carnitin, N-acetyl-D-glucosamine, NAD⁺, NADH, NADP⁺, N-decanoylglycine, phosphoribosyl-AMP, purine, or tiglylcarnitine. The detecting can include LC-MS, NMR, GC-MS, CE-MS, FTIR, or combinations thereof. The mammal can be a human. The sample can be whole blood, serum, plasma, urine, CSF, saliva, jejunum tissue, lung tissue, heart tissue, kidney tissue, skin tissue, bone marrow, gastrointestinal tract tissue, cardiovascular system tissue, CNS tissue, hematopoietic cells, or a fecal sample. The cancer treatment can include administering an anti-cancer agent selected from the group consisting of a chemotherapeutic agent, a targeted cancer drug, an immunotherapy drug, and a hormone therapy drug. The cancer treatment can include surgery.

In another aspect, this document features methods for treating a mammal having cancer. The methods can include, or consist essentially of, (a) detecting a presence of at least one enriched metabolic pathway in a sample from a mammal having cancer; and (b) administering a cancer treatment to the mammal, where the cancer treatment is not a radiation therapy. The at least one enriched metabolic pathway can be Warburg effect, arginine and proline metabolism, glutamate metabolism, pyruvate metabolism, fatty acid metabolism, propanoate metabolism, nicotinate and nicotinamide metabolism, gluconeogenesis, ammonia recycling, pyrimidine metabolism, urea cycle, selenoamino acid metabolism, mitochondrial beta-oxidation of long chain saturated fatty acids, mitochondrial beta-oxidation of short chain saturated fatty acids, cysteine metabolism, oxidation of branched chain fatty acids, glycolysis, purine metabolism, carnitine synthesis, glutathione metabolism, lactose synthesis, beta oxidation of very long chain fatty acids, or valine, leucine and isoleucine degradation. The mammal can be a human. The sample can be whole blood, serum, plasma, urine, CSF, saliva, jejunum tissue, lung tissue, heart tissue, kidney tissue, skin tissue, bone marrow, gastrointestinal tract tissue, cardiovascular system tissue, CNS tissue, hematopoietic cells, or a fecal sample. The cancer treatment can include administering an anti-cancer agent selected from the group consisting of a chemotherapeutic agent, a targeted cancer drug, an immunotherapy drug, and a hormone therapy drug. The cancer treatment can include surgery.

In another aspect, this document features methods for treating a mammal. The methods can include, or consist essentially of, (a) identifying a mammal as being likely to develop a radiation injury following radiation exposure by detecting a presence of an altered level of at least five metabolites in a sample from the mammal; and (b) administering a radioprotective agent or radio-mitigation agent to the mammal. The altered level of the at least five metabolites can be an increased level, and the at least five metabolites can be 10,11-dihydro-12r-hydroxy-leukotriene E4, 12-oxo-20-trihydroxy-leukotriene B4, 2-amino-3-phosphonopropionic acid, 3-hydroxybutyrate, 5-hydroxyindoleacetic acid, 5-hydroxytryptophol glucuronide, 7-methylguanosine, acetyldigitoxin, alpha-CEHC, D-glucose, formamidopyrimidine nucleoside triphosphate, gamma glutamylglutamic acid, gamma-glutamylthreonine, ganglioside GA1 (d18:1/16:0), ganglioside GM2 (d18:1/18:1(11z)), L-beta-aspartyl-L-threonine, leucyl-valine, leukotriene F4, L-isoleucine, L-leucine, lysoPC(22:2(13z,16z)), N-a-acetyl-L-arginine, N-acetyltryptophan, N-gamma-L-glutamyl-D-alanine, PG(18:3(9Z,12Z,15Z)/20:3(5Z,8Z,11Z)), phenylalanylphenylalanine, phosphoribosyl-AMP, p-hydroxyphenylacetic acid, PS(14:0/14:1(9Z)), PS(20:4(5Z,8Z,11Z,14Z)/14:1(9Z)), pyruvate, tetrahexosylceramide (d18:1/16:0), trans-3-hydroxycotinine glucuronide, trimethylamine N-oxide, uridine, valine, vanylglycol, L-carnitine, tiglyl-CoA, 2,2-dimethylsuccinic acid, 3-methyl-2-oxovalerate, anserine, L-phenylalanine, L-tryptophan, N,N-Dimethylglycine, N6-acetyllysine, N-acetylysteine, propylene glycol, undecanoic acid, or xanthurenate. The altered level of the at least five metabolites can be a decreased level, and the at least five metabolites can be 1-methylhistidine, 2-aminomuconic acid semialdehyde, 2-methylguanosine, 2-oxoarginine, 2-pyrroloylglycine, 3-(3,5-diiodo-4-hydroxyphenyl)pyruvate, 3-dehydroxycarnitine, 3-hydroxyoctanoyl carnitine, 3-nitrotyrosine, 3′-O-methyladenosine, 4-guanidinobutanoic acid, 7-methylguanine, alanyl-serine, allantoic acid, argininic acid, arginyl-methionine, citric acid, creatine, cysteic acid, cysteinyl-cysteine, DL-2-aminooctanoic acid, dUMP, gamma-glutamyltyrosine, glutaminyl-lysine, glutarylcarnitine, glycerophosphocholine, glyceryl lactopalmitate, hippuric acid, IDP, L-acetylcarnitine, L-alanine, L-aspartyl-4-phosphate, L-carnitine, L-glutamine, L-histidine, L-isoleucine, L-phenylalanine, L-tyrosine, melatonin, N1-methyl-2-pyridone-5-carboxamide, N6,N6,N6-trimethyl-L-lysine, N-acetyl-L-methionine, nicotine imine, N-ribosylhistidine, O-isobutyryl-L-carnitine, phenol sulphate, phenylpyruvic acid, picolinoylglycine, proline betaine, pyrogallol-2-O-glucuronide, riboflavin, serinyl-hydroxyproline, sphinganine 1-phosphate, trans-3-coumarate, tyrosyl-glutamate, urocanic acid, 1H-indole-3-carboxaldehyde, 2-hydroxyadenine, allodesmosine, arginyl-histidine, guanine, hypoxanthine, LysoPC(14:0), LysoPC(15:0), LysoPC(18:3(6Z,9Z,12Z)), LysoPE(0:0/18:1(11Z)), phosphoric acid, pyrrolidine, uridine, 1,9-dimethylurate, 2-(3-carboxy-3-aminopropyl)-L-histidine, adenosine diphosphate ribose, adenosine thiamine triphosphate, ADP, ATP, cholesterol glucuronide, D-galactose, D-glucose, GTP, malonyl-carnitin, N-acetyl-D-glucosamine, NAD⁺, NADH, NADP⁺, N-decanoylglycine, phosphoribosyl-AMP, purine, or tiglylcarnitine. The detecting can include LC-MS, NMR, GC-MS, CE-MS, FTIR, or combinations thereof. The method of any one of claims 32-37, wherein said mammal is a human. The sample can be whole blood, serum, plasma, urine, CSF, saliva, jejunum tissue, lung tissue, heart tissue, kidney tissue, skin tissue, bone marrow, gastrointestinal tract tissue, cardiovascular system tissue, CNS tissue, hematopoietic cells, or a fecal sample. The radioprotective agent can be amifostine (2-(3-aminpropyl) aminoethylphosphorothioate), potassium iodide (KI), Prussian blue, diethylenetriamine pentaacetate (DTPA), or filgrastim. The radiation exposure can include radiation therapy. The mammal can have cancer. The radiation exposure can occur during an event selected from the group consisting of an industrial accident, terrorist attack, military action, commercial exposure, medical procedure, environmental exposure, and any combinations thereof. The radiation exposure can include a radiation source selected from the group consisting of ionizing radiation, non-ionizing radiation, low energy particles, and high energy particles. The radiation injury can include a CRI and/or ARS.

In another aspect, this document features methods for treating a mammal. The methods can include, or consist essentially of, (a) identifying a mammal as being likely to develop a radiation injury following radiation exposure by detecting a presence of at least one enriched metabolic pathway in a sample from the mammal; and (b) administering a radioprotective agent or a radio-mitigation agent to the mammal. The at least one enriched metabolic pathway can be Warburg effect, arginine and proline metabolism, glutamate metabolism, pyruvate metabolism, fatty acid metabolism, propanoate metabolism, nicotinate and nicotinamide metabolism, gluconeogenesis, ammonia recycling, pyrimidine metabolism, urea cycle, selenoamino acid metabolism, mitochondrial beta-oxidation of long chain saturated fatty acids, mitochondrial beta-oxidation of short chain saturated fatty acids, cysteine metabolism, oxidation of branched chain fatty acids, glycolysis, purine metabolism, carnitine synthesis, glutathione metabolism, lactose synthesis, beta oxidation of very long chain fatty acids, or valine, leucine and isoleucine degradation. The method of any one of claims 32-37, wherein said mammal is a human. The sample can be whole blood, serum, plasma, urine, CSF, saliva, jejunum tissue, lung tissue, heart tissue, kidney tissue, skin tissue, bone marrow, gastrointestinal tract tissue, cardiovascular system tissue, CNS tissue, hematopoietic cells, or a fecal sample. The radioprotective agent can be amifostine (2-(3-aminpropyl) aminoethylphosphorothioate), potassium iodide (KI), Prussian blue, diethylenetriamine pentaacetate (DTPA), or filgrastim. The radiation exposure can include radiation therapy. The mammal can have cancer. The radiation exposure can occur during an event selected from the group consisting of an industrial accident, terrorist attack, military action, commercial exposure, medical procedure, environmental exposure, and any combinations thereof. The radiation exposure can include a radiation source selected from the group consisting of ionizing radiation, non-ionizing radiation, low energy particles, and high energy particles. The radiation injury can include a CRI and/or ARS.

In another aspect, this document features methods for monitoring a mammal for radiation injury following radiation exposure. The methods can include, or consist essentially of, (a) detecting a presence or absence of an altered level of at least five metabolites in a sample from a mammal having been exposed to radiation; (b) classifying the mammal as being likely to develop a radiation injury following radiation exposure if the presence of the altered level is detected; and (c) classifying the mammal as not being likely to develop a radiation injury if the presence of the altered level is not detected. The altered level of the at least five metabolites can be an increased level, and the at least five metabolites can be 10,11-dihydro-12r-hydroxy-leukotriene E4, 12-oxo-20-trihydroxy-leukotriene B4, 2-amino-3-phosphonopropionic acid, 3-hydroxybutyrate, 5-hydroxyindoleacetic acid, 5-hydroxytryptophol glucuronide, 7-methylguanosine, acetyldigitoxin, alpha-CEHC, D-glucose, formamidopyrimidine nucleoside triphosphate, gamma glutamylglutamic acid, gamma-glutamylthreonine, ganglioside GA1 (d18:1/16:0), ganglioside GM2 (d18:1/18:1(11z)), L-beta-aspartyl-L-threonine, leucyl-valine, leukotriene F4, L-isoleucine, L-leucine, lysoPC(22:2(13z,16z)), N-a-acetyl-L-arginine, N-acetyltryptophan, N-gamma-L-glutamyl-D-alanine, PG(18:3(9Z,12Z,15Z)/20:3(5Z,8Z,11Z)), phenylalanylphenylalanine, phosphoribosyl-AMP, p-hydroxyphenylacetic acid, PS(14:0/14:1(9Z)), PS(20:4(5Z,8Z,11Z,14Z)/14:1(9Z)), pyruvate, tetrahexosylceramide (d18:1/16:0), trans-3-hydroxycotinine glucuronide, trimethylamine N-oxide, uridine, valine, vanylglycol, L-carnitine, tiglyl-CoA, 2,2-dimethylsuccinic acid, 3-methyl-2-oxovalerate, anserine, L-phenylalanine, L-tryptophan, N,N-Dimethylglycine, N6-acetyllysine, N-acetylysteine, propylene glycol, undecanoic acid, or xanthurenate. The altered level of the at least five metabolites can be a decreased level, and the at least five metabolites can be 1-methylhistidine, 2-aminomuconic acid semialdehyde, 2-methylguanosine, 2-oxoarginine, 2-pyrroloylglycine, 3-(3,5-diiodo-4-hydroxyphenyl)pyruvate, 3-dehydroxycarnitine, 3-hydroxyoctanoyl carnitine, 3-nitrotyrosine, 3′-O-methyladenosine, 4-guanidinobutanoic acid, 7-methylguanine, alanyl-serine, allantoic acid, argininic acid, arginyl-methionine, citric acid, creatine, cysteic acid, cysteinyl-cysteine, DL-2-aminooctanoic acid, dUMP, gamma-glutamyltyrosine, glutaminyl-lysine, glutarylcarnitine, glycerophosphocholine, glyceryl lactopalmitate, hippuric acid, IDP, L-acetylcarnitine, L-alanine, L-aspartyl-4-phosphate, L-carnitine, L-glutamine, L-histidine, L-isoleucine, L-phenylalanine, L-tyrosine, melatonin, N1-methyl-2-pyridone-5-carboxamide, N6,N6,N6-trimethyl-L-lysine, N-acetyl-L-methionine, nicotine imine, N-ribosylhistidine, O-isobutyryl-L-carnitine, phenol sulphate, phenylpyruvic acid, picolinoylglycine, proline betaine, pyrogallol-2-O-glucuronide, riboflavin, serinyl-hydroxyproline, sphinganine 1-phosphate, trans-3-coumarate, tyrosyl-glutamate, urocanic acid, 1H-indole-3-carboxaldehyde, 2-hydroxyadenine, allodesmosine, arginyl-histidine, guanine, hypoxanthine, LysoPC(14:0), LysoPC(15:0), LysoPC(18:3(6Z,9Z,12Z)), LysoPE(0:0/18:1(11Z)), phosphoric acid, pyrrolidine, uridine, 1,9-dimethylurate, 2-(3-carboxy-3-aminopropyl)-L-histidine, adenosine diphosphate ribose, adenosine thiamine triphosphate, ADP, ATP, cholesterol glucuronide, D-galactose, D-glucose, GTP, malonyl-carnitin, N-acetyl-D-glucosamine, NAD+, NADH, NADP+, N-decanoylglycine, phosphoribosyl-AMP, purine, or tiglylcarnitine. The detecting can include LC-MS, NMR, GC-MS, CE-MS, FTIR, or combinations thereof. The mammal can be a human. The sample can be whole blood, serum, plasma, urine, CSF, saliva, jejunum tissue, lung tissue, heart tissue, kidney tissue, skin tissue, bone marrow, gastrointestinal tract tissue, cardiovascular system tissue, CNS tissue, hematopoietic cells, or a fecal sample. The radiation exposure can include radiation therapy. The mammal can have cancer. The radiation exposure can occur during an event selected from the group consisting of an industrial accident, terrorist attack, military action, commercial exposure, medical procedure, environmental exposure, and any combinations thereof. The radiation exposure can include a radiation source selected from the group consisting of ionizing radiation, non-ionizing radiation, low energy particles, and high energy particles. The radiation injury can include a CRI and/or ARS.

In another aspect, this document features methods for monitoring a mammal for radiation injury following radiation exposure. The methods can include, or consist essentially of, (a) detecting a presence or absence of at least one enriched metabolic pathway in a sample from a mammal having been exposed to radiation; (b) classifying the mammal as being likely to develop a radiation injury following radiation exposure if the presence of said enriched metabolic pathways is detected; and (c) classifying the mammal as not being likely to develop a radiation injury if the presence of the enriched metabolic pathways is not detected. The at least one enriched metabolic pathway can be Warburg effect, arginine and proline metabolism, glutamate metabolism, pyruvate metabolism, fatty acid metabolism, propanoate metabolism, nicotinate and nicotinamide metabolism, gluconeogenesis, ammonia recycling, pyrimidine metabolism, urea cycle, selenoamino acid metabolism, mitochondrial beta-oxidation of long chain saturated fatty acids, mitochondrial beta-oxidation of short chain saturated fatty acids, cysteine metabolism, oxidation of branched chain fatty acids, glycolysis, purine metabolism, carnitine synthesis, glutathione metabolism, lactose synthesis, beta oxidation of very long chain fatty acids, or valine, leucine and isoleucine degradation. The mammal can be a human. The sample can be whole blood, serum, plasma, urine, CSF, saliva, jejunum tissue, lung tissue, heart tissue, kidney tissue, skin tissue, bone marrow, gastrointestinal tract tissue, cardiovascular system tissue, CNS tissue, hematopoietic cells, or a fecal sample. The radiation exposure can include radiation therapy. The mammal can have cancer. The radiation exposure can occur during an event selected from the group consisting of an industrial accident, terrorist attack, military action, commercial exposure, medical procedure, environmental exposure, and any combinations thereof. The radiation exposure can include a radiation source selected from the group consisting of ionizing radiation, non-ionizing radiation, low energy particles, and high energy particles. The radiation injury can include a CRI and/or ARS.

In another aspect, this document features methods for identifying a radioprotective agent. The methods can include, or consist essentially of, (a) subjecting a non-human mammal to radiation exposure; (b) administering a candidate compound to the non-human mammal; (c) detecting a presence or absence of an altered level of at least five metabolites in a sample from the non-human mammal; (d) not classifying the candidate compound as a radioprotective agent if the presence of the altered level is detected; and (e) classifying the candidate compound as a radioprotective agent if the presence of the altered level is not detected. The altered level of the at least five metabolites can be an increased level, and the at least five metabolites can be 10,11-dihydro-12r-hydroxy-leukotriene E4, 12-oxo-20-trihydroxy-leukotriene B4, 2-amino-3-phosphonopropionic acid, 3-hydroxybutyrate, 5-hydroxyindoleacetic acid, 5-hydroxytryptophol glucuronide, 7-methylguanosine, acetyldigitoxin, alpha-CEHC, D-glucose, formamidopyrimidine nucleoside triphosphate, gamma glutamylglutamic acid, gamma-glutamylthreonine, ganglioside GA1 (d18:1/16:0), ganglioside GM2 (d18:1/18:1(11z)), L-beta-aspartyl-L-threonine, leucyl-valine, leukotriene F4, L-isoleucine, L-leucine, lysoPC(22:2(13z,16z)), N-a-acetyl-L-arginine, N-acetyltryptophan, N-gamma-L-glutamyl-D-alanine, PG(18:3(9Z,12Z,15Z)/20:3(5Z,8Z,11Z)), phenylalanylphenylalanine, phosphoribosyl-AMP, p-hydroxyphenylacetic acid, PS(14:0/14:1(9Z)), PS(20:4(5Z,8Z,11Z,14Z)/14:1(9Z)), pyruvate, tetrahexosylceramide (d18:1/16:0), trans-3-hydroxycotinine glucuronide, trimethylamine N-oxide, uridine, valine, vanylglycol, L-carnitine, tiglyl-CoA, 2,2-dimethylsuccinic acid, 3-methyl-2-oxovalerate, anserine, L-phenylalanine, L-tryptophan, N,N-Dimethylglycine, N6-acetyllysine, N-acetylysteine, propylene glycol, undecanoic acid, or xanthurenate. The altered level of the at least five metabolites can be a decreased level, and the at least five metabolites can be 1-methylhistidine, 2-aminomuconic acid semialdehyde, 2-methylguanosine, 2-oxoarginine, 2-pyrroloylglycine, 3-(3,5-diiodo-4-hydroxyphenyl)pyruvate, 3-dehydroxycarnitine, 3-hydroxyoctanoyl carnitine, 3-nitrotyrosine, 3′-O-methyladenosine, 4-guanidinobutanoic acid, 7-methylguanine, alanyl-serine, allantoic acid, argininic acid, arginyl-methionine, citric acid, creatine, cysteic acid, cysteinyl-cysteine, DL-2-aminooctanoic acid, dUMP, gamma-glutamyltyrosine, glutaminyl-lysine, glutarylcarnitine, glycerophosphocholine, glyceryl lactopalmitate, hippuric acid, IDP, L-acetylcarnitine, L-alanine, L-aspartyl-4-phosphate, L-carnitine, L-glutamine, L-histidine, L-isoleucine, L-phenylalanine, L-tyrosine, melatonin, N1-methyl-2-pyridone-5-carboxamide, N6,N6,N6-trimethyl-L-lysine, N-acetyl-L-methionine, nicotine imine, N-ribosylhistidine, 0-isobutyryl-L-carnitine, phenol sulphate, phenylpyruvic acid, picolinoylglycine, proline betaine, pyrogallol-2-O-glucuronide, riboflavin, serinyl-hydroxyproline, sphinganine 1-phosphate, trans-3-coumarate, tyrosyl-glutamate, urocanic acid, 1H-indole-3-carboxaldehyde, 2-hydroxyadenine, allodesmosine, arginyl-histidine, guanine, hypoxanthine, LysoPC(14:0), LysoPC(15:0), LysoPC(18:3(6Z,9Z,12Z)), LysoPE(0:0/18:1(11Z)), phosphoric acid, pyrrolidine, uridine, 1,9-dimethylurate, 2-(3-carboxy-3-aminopropyl)-L-histidine, adenosine diphosphate ribose, adenosine thiamine triphosphate, ADP, ATP, cholesterol glucuronide, D-galactose, D-glucose, GTP, malonyl-camitin, N-acetyl-D-glucosamine, NAD⁺, NADH, NADP⁺, N-decanoylglycine, phosphoribosyl-AMP, purine, or tiglylcarnitine. The detecting can include LC-MS, NMR, GC-MS, CE-MS, FTIR, or combinations thereof. The non-human mammal can be a non-human primate. The sample can be whole blood, serum, plasma, urine, CSF, saliva, jejunum tissue, lung tissue, heart tissue, kidney tissue, skin tissue, bone marrow, gastrointestinal tract tissue, cardiovascular system tissue, CNS tissue, hematopoietic cells, or a fecal sample. The radiation exposure can occur during an event selected from the group consisting of an industrial accident, terrorist attack, military action, commercial exposure, medical procedure, environmental exposure, and any combinations thereof. The radiation exposure can include a radiation source selected from the group consisting of ionizing radiation, non-ionizing radiation, low energy particles, and high energy particles.

In another aspect, this document features methods for identifying a radioprotective agent. The methods can include, or consist essentially of, (a) subjecting a non-human mammal to radiation exposure; (b) administering a candidate compound to the non-human mammal; (c) detecting a presence or absence of at least one enriched metabolic pathway in a sample from the non-human mammal; (d) not classifying said candidate compound as a radioprotective agent if the presence of the enriched metabolic pathways is detected; and (e) classifying the candidate compound as a radioprotective agent if the presence of the enriched metabolic pathways is not detected. The at least one enriched metabolic pathway can be Warburg effect, arginine and proline metabolism, glutamate metabolism, pyruvate metabolism, fatty acid metabolism, propanoate metabolism, nicotinate and nicotinamide metabolism, gluconeogenesis, ammonia recycling, pyrimidine metabolism, urea cycle, selenoamino acid metabolism, mitochondrial beta-oxidation of long chain saturated fatty acids, mitochondrial beta-oxidation of short chain saturated fatty acids, cysteine metabolism, oxidation of branched chain fatty acids, glycolysis, purine metabolism, carnitine synthesis, glutathione metabolism, lactose synthesis, beta oxidation of very long chain fatty acids, or valine, leucine and isoleucine degradation. The non-human mammal can be a non-human primate. The sample can be whole blood, serum, plasma, urine, CSF, saliva, jejunum tissue, lung tissue, heart tissue, kidney tissue, skin tissue, bone marrow, gastrointestinal tract tissue, cardiovascular system tissue, CNS tissue, hematopoietic cells, or a fecal sample. The radiation exposure can occur during an event selected from the group consisting of an industrial accident, terrorist attack, military action, commercial exposure, medical procedure, environmental exposure, and any combinations thereof. The radiation exposure can include a radiation source selected from the group consisting of ionizing radiation, non-ionizing radiation, low energy particles, and high energy particles.

In another aspect, this document features methods for monitoring a mammal having been administered a radiation counter measure following radiation exposure. The methods can include, or consist essentially of, (a) detecting a presence or absence of an altered level of at least five metabolites in a sample from a mammal having been administered a radiation counter measure following radiation exposure; (b) classifying the radiation counter measure as being an effective treatment for the mammal if the presence of the altered level is not detected; and (c) classifying the radiation counter measure as not being an effective treatment for the mammal if the presence of the altered level is detected. The altered level of the at least five metabolites can be an increased level, and the at least five metabolites can be 10,11-dihydro-12r-hydroxy-leukotriene E4, 12-oxo-20-trihydroxy-leukotriene B4, 2-amino-3-phosphonopropionic acid, 3-hydroxybutyrate, 5-hydroxyindoleacetic acid, 5-hydroxytryptophol glucuronide, 7-methylguanosine, acetyldigitoxin, alpha-CEHC, D-glucose, formamidopyrimidine nucleoside triphosphate, gamma glutamylglutamic acid, gamma-glutamylthreonine, ganglioside GA1 (d18:1/16:0), ganglioside GM2 (d18:1/18:1(11z)), L-beta-aspartyl-L-threonine, leucyl-valine, leukotriene F4, L-isoleucine, L-leucine, lysoPC(22:2(13z,16z)), N-a-acetyl-L-arginine, N-acetyltryptophan, N-gamma-L-glutamyl-D-alanine, PG(18:3(9Z,12Z,15Z)/20:3(5Z,8Z,11Z)), phenylalanylphenylalanine, phosphoribosyl-AMP, p-hydroxyphenylacetic acid, PS(14:0/14:1(9Z)), PS(20:4(5Z,8Z,11Z,14Z)/14:1(9Z)), pyruvate, tetrahexosylceramide (d18:1/16:0), trans-3-hydroxycotinine glucuronide, trimethylamine N-oxide, uridine, valine, vanylglycol, L-carnitine, tiglyl-CoA, 2,2-dimethylsuccinic acid, 3-methyl-2-oxovalerate, anserine, L-phenylalanine, L-tryptophan, N,N-Dimethylglycine, N6-acetyllysine, N-acetylysteine, propylene glycol, undecanoic acid, or xanthurenate. The altered level of the at least five metabolites can be a decreased level, and the at least five metabolites can be 1-methylhistidine, 2-aminomuconic acid semialdehyde, 2-methylguanosine, 2-oxoarginine, 2-pyrroloylglycine, 3-(3,5-diiodo-4-hydroxyphenyl)pyruvate, 3-dehydroxycarnitine, 3-hydroxyoctanoyl carnitine, 3-nitrotyrosine, 3′-O-methyladenosine, 4-guanidinobutanoic acid, 7-methylguanine, alanyl-serine, allantoic acid, argininic acid, arginyl-methionine, citric acid, creatine, cysteic acid, cysteinyl-cysteine, DL-2-aminooctanoic acid, dUMP, gamma-glutamyltyrosine, glutaminyl-lysine, glutarylcarnitine, glycerophosphocholine, glyceryl lactopalmitate, hippuric acid, IDP, L-acetylcarnitine, L-alanine, L-aspartyl-4-phosphate, L-carnitine, L-glutamine, L-histidine, L-isoleucine, L-phenylalanine, L-tyrosine, melatonin, N1-methyl-2-pyridone-5-carboxamide, N6,N6,N6-trimethyl-L-lysine, N-acetyl-L-methionine, nicotine imine, N-ribosylhistidine, 0-isobutyryl-L-carnitine, phenol sulphate, phenylpyruvic acid, picolinoylglycine, proline betaine, pyrogallol-2-O-glucuronide, riboflavin, serinyl-hydroxyproline, sphinganine 1-phosphate, trans-3-coumarate, tyrosyl-glutamate, urocanic acid, 1H-indole-3-carboxaldehyde, 2-hydroxyadenine, allodesmosine, arginyl-histidine, guanine, hypoxanthine, LysoPC(14:0), LysoPC(15:0), LysoPC(18:3(6Z,9Z,12Z)), LysoPE(0:0/18:1(11Z)), phosphoric acid, pyrrolidine, uridine, 1,9-dimethylurate, 2-(3-carboxy-3-aminopropyl)-L-histidine, adenosine diphosphate ribose, adenosine thiamine triphosphate, ADP, ATP, cholesterol glucuronide, D-galactose, D-glucose, GTP, malonyl-camitin, N-acetyl-D-glucosamine, NAD⁺, NADH, NADP⁺, N-decanoylglycine, phosphoribosyl-AMP, purine, or tiglylcarnitine. The detecting can include LC-MS, NMR, GC-MS, CE-MS, FTIR, or combinations thereof. The mammal can be a human. The sample can be whole blood, serum, plasma, urine, CSF, saliva, jejunum tissue, lung tissue, heart tissue, kidney tissue, skin tissue, bone marrow, gastrointestinal tract tissue, cardiovascular system tissue, CNS tissue, hematopoietic cells, or a fecal sample. The radiation exposure can occur during an event selected from the group consisting of an industrial accident, terrorist attack, military action, commercial exposure, medical procedure, environmental exposure, and any combinations thereof. The radiation exposure can include a radiation source selected from the group consisting of ionizing radiation, non-ionizing radiation, low energy particles, and high energy particles.

In another aspect, this document features methods for monitoring a mammal having been administered a radiation counter measure following radiation exposure. The methods can include, or consist essentially of, (a) detecting a presence or absence of at least one enriched metabolic pathway in a sample from a mammal having been administered a radiation counter measure following radiation exposure; (b) classifying the radiation counter measure as being an effective treatment for the mammal if the presence of the enriched metabolic pathways is not detected; and (c) classifying the radiation counter measure as not being an effective treatment for the mammal if the presence of the enriched metabolic pathways is detected. The at least one enriched metabolic pathway can be Warburg effect, arginine and proline metabolism, glutamate metabolism, pyruvate metabolism, fatty acid metabolism, propanoate metabolism, nicotinate and nicotinamide metabolism, gluconeogenesis, ammonia recycling, pyrimidine metabolism, urea cycle, selenoamino acid metabolism, mitochondrial beta-oxidation of long chain saturated fatty acids, mitochondrial beta-oxidation of short chain saturated fatty acids, cysteine metabolism, oxidation of branched chain fatty acids, glycolysis, purine metabolism, carnitine synthesis, glutathione metabolism, lactose synthesis, beta oxidation of very long chain fatty acids, or valine, leucine and isoleucine degradation. The mammal can be a human. The sample can be whole blood, serum, plasma, urine, CSF, saliva, jejunum tissue, lung tissue, heart tissue, kidney tissue, skin tissue, bone marrow, gastrointestinal tract tissue, cardiovascular system tissue, CNS tissue, hematopoietic cells, or a fecal sample. The radiation exposure can occur during an event selected from the group consisting of an industrial accident, terrorist attack, military action, commercial exposure, medical procedure, environmental exposure, and any combinations thereof. The radiation exposure can include a radiation source selected from the group consisting of ionizing radiation, non-ionizing radiation, low energy particles, and high energy particles.

In another aspect, this document features methods for monitoring a mammal having been administered a radiation counter measure following radiation exposure. The methods can include, or consist essentially of, (a) identifying a mammal as having been exposed to radiation; (b) administering a radiation counter measure to the mammal; (c) detecting a presence or absence of an altered level of at least five metabolites in a sample from the mammal; (d) classifying the radiation counter measure as being an effective treatment for the mammal if the presence of the altered level is not detected; and (e) classifying the radiation counter measure as not being an effective treatment for said mammal if the presence of the altered level is detected. The altered level of the at least five metabolites can be an increased level, and the at least five metabolites can be 10,11-dihydro-12r-hydroxy-leukotriene E4, 12-oxo-20-trihydroxy-leukotriene B4, 2-amino-3-phosphonopropionic acid, 3-hydroxybutyrate, 5-hydroxyindoleacetic acid, 5-hydroxytryptophol glucuronide, 7-methylguanosine, acetyldigitoxin, alpha-CEHC, D-glucose, formamidopyrimidine nucleoside triphosphate, gamma glutamylglutamic acid, gamma-glutamylthreonine, ganglioside GA1 (d18:1/16:0), ganglioside GM2 (d18:1/18:1(11z)), L-beta-aspartyl-L-threonine, leucyl-valine, leukotriene F4, L-isoleucine, L-leucine, lysoPC(22:2(13z,16z)), N-a-acetyl-L-arginine, N-acetyltryptophan, N-gamma-L-glutamyl-D-alanine, PG(18:3(9Z,12Z,15Z)/20:3(5Z,8Z,11Z)), phenylalanylphenylalanine, phosphoribosyl-AMP, p-hydroxyphenylacetic acid, PS(14:0/14:1(9Z)), PS(20:4(5Z,8Z,11Z,14Z)/14:1(9Z)), pyruvate, tetrahexosylceramide (d18:1/16:0), trans-3-hydroxycotinine glucuronide, trimethylamine N-oxide, uridine, valine, vanylglycol, L-carnitine, tiglyl-CoA, 2,2-dimethylsuccinic acid, 3-methyl-2-oxovalerate, anserine, L-phenylalanine, L-tryptophan, N,N-Dimethylglycine, N6-acetyllysine, N-acetylysteine, propylene glycol, undecanoic acid, or xanthurenate. The altered level of the at least five metabolites can be a decreased level, and the at least five metabolites can be 1-methylhistidine, 2-aminomuconic acid semialdehyde, 2-methylguanosine, 2-oxoarginine, 2-pyrroloylglycine, 3-(3,5-diiodo-4-hydroxyphenyl)pyruvate, 3-dehydroxycarnitine, 3-hydroxyoctanoyl carnitine, 3-nitrotyrosine, 3′-O-methyladenosine, 4-guanidinobutanoic acid, 7-methylguanine, alanyl-serine, allantoic acid, argininic acid, arginyl-methionine, citric acid, creatine, cysteic acid, cysteinyl-cysteine, DL-2-aminooctanoic acid, dUMP, gamma-glutamyltyrosine, glutaminyl-lysine, glutarylcarnitine, glycerophosphocholine, glyceryl lactopalmitate, hippuric acid, IDP, L-acetylcarnitine, L-alanine, L-aspartyl-4-phosphate, L-carnitine, L-glutamine, L-histidine, L-isoleucine, L-phenylalanine, L-tyrosine, melatonin, N1-methyl-2-pyridone-5-carboxamide, N6,N6,N6-trimethyl-L-lysine, N-acetyl-L-methionine, nicotine imine, N-ribosylhistidine, O-isobutyryl-L-carnitine, phenol sulphate, phenylpyruvic acid, picolinoylglycine, proline betaine, pyrogallol-2-O-glucuronide, riboflavin, serinyl-hydroxyproline, sphinganine 1-phosphate, trans-3-coumarate, tyrosyl-glutamate, urocanic acid, 1H-indole-3-carboxaldehyde, 2-hydroxyadenine, allodesmosine, arginyl-histidine, guanine, hypoxanthine, LysoPC(14:0), LysoPC(15:0), LysoPC(18:3(6Z,9Z,12Z)), LysoPE(0:0/18:1(11Z)), phosphoric acid, pyrrolidine, uridine, 1,9-dimethylurate, 2-(3-carboxy-3-aminopropyl)-L-histidine, adenosine diphosphate ribose, adenosine thiamine triphosphate, ADP, ATP, cholesterol glucuronide, D-galactose, D-glucose, GTP, malonyl-carnitin, N-acetyl-D-glucosamine, NAD⁺, NADH, NADP⁺, N-decanoylglycine, phosphoribosyl-AMP, purine, or tiglylcarnitine. The detecting can include LC-MS, NMR, GC-MS, CE-MS, FTIR, or combinations thereof. The mammal can be a human. The sample can be whole blood, serum, plasma, urine, CSF, saliva, jejunum tissue, lung tissue, heart tissue, kidney tissue, skin tissue, bone marrow, gastrointestinal tract tissue, cardiovascular system tissue, CNS tissue, hematopoietic cells, or a fecal sample. The radiation exposure can occur during an event selected from the group consisting of an industrial accident, terrorist attack, military action, commercial exposure, medical procedure, environmental exposure, and any combinations thereof. The radiation exposure can include a radiation source selected from the group consisting of ionizing radiation, non-ionizing radiation, low energy particles, and high energy particles.

In another aspect, this document features methods for monitoring a mammal having been administered a radiation counter measure following radiation exposure. The methods can include, or consist essentially of, (a) identifying a mammal as having been exposed to radiation; (b) administering a radiation counter measure to the mammal; (c) detecting a presence or absence of at least one enriched metabolic pathway in a sample from the mammal; (d) classifying the radiation counter measure as being an effective treatment for the mammal if the presence of the enriched metabolic pathways is not detected; and (e) classifying the radiation counter measure as not being an effective treatment for the mammal if said presence of the enriched metabolic pathways is detected. The at least one enriched metabolic pathway can be Warburg effect, arginine and proline metabolism, glutamate metabolism, pyruvate metabolism, fatty acid metabolism, propanoate metabolism, nicotinate and nicotinamide metabolism, gluconeogenesis, ammonia recycling, pyrimidine metabolism, urea cycle, selenoamino acid metabolism, mitochondrial beta-oxidation of long chain saturated fatty acids, mitochondrial beta-oxidation of short chain saturated fatty acids, cysteine metabolism, oxidation of branched chain fatty acids, glycolysis, purine metabolism, carnitine synthesis, glutathione metabolism, lactose synthesis, beta oxidation of very long chain fatty acids, or valine, leucine and isoleucine degradation. The mammal can be a human. The sample can be whole blood, serum, plasma, urine, CSF, saliva, jejunum tissue, lung tissue, heart tissue, kidney tissue, skin tissue, bone marrow, gastrointestinal tract tissue, cardiovascular system tissue, CNS tissue, hematopoietic cells, or a fecal sample. The radiation exposure can occur during an event selected from the group consisting of an industrial accident, terrorist attack, military action, commercial exposure, medical procedure, environmental exposure, and any combinations thereof. The radiation exposure can include a radiation source selected from the group consisting of ionizing radiation, non-ionizing radiation, low energy particles, and high energy particles.

In another aspect, this document features methods for monitoring a mammal having been administered a radiation counter measure following radiation exposure. The methods can include, or consist essentially of, (a) detecting a presence or absence of an altered level of at least five metabolites in a sample from a mammal having been administered a radiation counter measure following radiation exposure mammal; (b) classifying the radiation counter measure as being an effective treatment for the mammal if the presence of said altered level is not detected; and (c) administering the radiation counter measure to the mammal. The altered level of the at least five metabolites can be an increased level, and the at least five metabolites can be 10,11-dihydro-12r-hydroxy-leukotriene E4, 12-oxo-20-trihydroxy-leukotriene B4, 2-amino-3-phosphonopropionic acid, 3-hydroxybutyrate, 5-hydroxyindoleacetic acid, 5-hydroxytryptophol glucuronide, 7-methylguanosine, acetyldigitoxin, alpha-CEHC, D-glucose, formamidopyrimidine nucleoside triphosphate, gamma glutamylglutamic acid, gamma-glutamylthreonine, ganglioside GA1 (d18:1/16:0), ganglioside GM2 (d18:1/18:1(11z)), L-beta-aspartyl-L-threonine, leucyl-valine, leukotriene F4, L-isoleucine, L-leucine, lysoPC(22:2(13z,16z)), N-a-acetyl-L-arginine, N-acetyltryptophan, N-gamma-L-glutamyl-D-alanine, PG(18:3(9Z,12Z,15Z)/20:3(5Z,8Z,11Z)), phenylalanylphenylalanine, phosphoribosyl-AMP, p-hydroxyphenylacetic acid, PS(14:0/14:1(9Z)), PS(20:4(5Z,8Z,11Z,14Z)/14:1(9Z)), pyruvate, tetrahexosylceramide (d18:1/16:0), trans-3-hydroxycotinine glucuronide, trimethylamine N-oxide, uridine, valine, vanylglycol, L-carnitine, tiglyl-CoA, 2,2-dimethylsuccinic acid, 3-methyl-2-oxovalerate, anserine, L-phenylalanine, L-tryptophan, N,N-Dimethylglycine, N6-acetyllysine, N-acetylysteine, propylene glycol, undecanoic acid, or xanthurenate. The altered level of the at least five metabolites can be a decreased level, and the at least five metabolites can be 1-methylhistidine, 2-aminomuconic acid semialdehyde, 2-methylguanosine, 2-oxoarginine, 2-pyrroloylglycine, 3-(3,5-diiodo-4-hydroxyphenyl)pyruvate, 3-dehydroxycarnitine, 3-hydroxyoctanoyl carnitine, 3-nitrotyrosine, 3′-O-methyladenosine, 4-guanidinobutanoic acid, 7-methylguanine, alanyl-serine, allantoic acid, argininic acid, arginyl-methionine, citric acid, creatine, cysteic acid, cysteinyl-cysteine, DL-2-aminooctanoic acid, dUMP, gamma-glutamyltyrosine, glutaminyl-lysine, glutarylcarnitine, glycerophosphocholine, glyceryl lactopalmitate, hippuric acid, IDP, L-acetylcarnitine, L-alanine, L-aspartyl-4-phosphate, L-carnitine, L-glutamine, L-histidine, L-isoleucine, L-phenylalanine, L-tyrosine, melatonin, N1-methyl-2-pyridone-5-carboxamide, N6,N6,N6-trimethyl-L-lysine, N-acetyl-L-methionine, nicotine imine, N-ribosylhistidine, O-isobutyryl-L-carnitine, phenol sulphate, phenylpyruvic acid, picolinoylglycine, proline betaine, pyrogallol-2-O-glucuronide, riboflavin, serinyl-hydroxyproline, sphinganine 1-phosphate, trans-3-coumarate, tyrosyl-glutamate, urocanic acid, 1H-indole-3-carboxaldehyde, 2-hydroxyadenine, allodesmosine, arginyl-histidine, guanine, hypoxanthine, LysoPC(14:0), LysoPC(15:0), LysoPC(18:3(6Z,9Z,12Z)), LysoPE(0:0/18:1(11Z)), phosphoric acid, pyrrolidine, uridine, 1,9-dimethylurate, 2-(3-carboxy-3-aminopropyl)-L-histidine, adenosine diphosphate ribose, adenosine thiamine triphosphate, ADP, ATP, cholesterol glucuronide, D-galactose, D-glucose, GTP, malonyl-carnitin, N-acetyl-D-glucosamine, NAD⁺, NADH, NADP⁺, N-decanoylglycine, phosphoribosyl-AMP, purine, or tiglylcarnitine. The detecting can include LC-MS, NMR, GC-MS, CE-MS, FTIR, or combinations thereof. The mammal can be a human. The sample can be whole blood, serum, plasma, urine, CSF, saliva, jejunum tissue, lung tissue, heart tissue, kidney tissue, skin tissue, bone marrow, gastrointestinal tract tissue, cardiovascular system tissue, CNS tissue, hematopoietic cells, or a fecal sample. The radiation exposure can occur during an event selected from the group consisting of an industrial accident, terrorist attack, military action, commercial exposure, medical procedure, environmental exposure, and any combinations thereof. The radiation exposure can include a radiation source selected from the group consisting of ionizing radiation, non-ionizing radiation, low energy particles, and high energy particles.

In another aspect, this document features methods for monitoring a mammal having been administered a first radiation counter measure following radiation exposure. The methods can include, or consist essentially of, (a) detecting a presence or absence of an altered level of at least five metabolites in a sample from a mammal having been administered a first radiation counter measure following radiation exposure mammal; (b) classifying the first radiation counter measure as not being an effective treatment for the mammal if the presence of the altered level is detected; and (c) administering a second radiation counter measure to the mammal. The altered level of the at least five metabolites can be an increased level, and the at least five metabolites can be 10,11-dihydro-12r-hydroxy-leukotriene E4, 12-oxo-20-trihydroxy-leukotriene B4, 2-amino-3-phosphonopropionic acid, 3-hydroxybutyrate, 5-hydroxyindoleacetic acid, 5-hydroxytryptophol glucuronide, 7-methylguanosine, acetyldigitoxin, alpha-CEHC, D-glucose, formamidopyrimidine nucleoside triphosphate, gamma glutamylglutamic acid, gamma-glutamylthreonine, ganglioside GA1 (d18:1/16:0), ganglioside GM2 (d18:1/18:1(11z)), L-beta-aspartyl-L-threonine, leucyl-valine, leukotriene F4, L-isoleucine, L-leucine, lysoPC(22:2(13z,16z)), N-a-acetyl-L-arginine, N-acetyltryptophan, N-gamma-L-glutamyl-D-alanine, PG(18:3(9Z,12Z,15Z)/20:3(5Z,8Z,11Z)), phenylalanylphenylalanine, phosphoribosyl-AMP, p-hydroxyphenylacetic acid, PS(14:0/14:1(9Z)), PS(20:4(5Z,8Z,11Z,14Z)/14:1(9Z)), pyruvate, tetrahexosylceramide (d18:1/16:0), trans-3-hydroxycotinine glucuronide, trimethylamine N-oxide, uridine, valine, vanylglycol, L-carnitine, tiglyl-CoA, 2,2-dimethylsuccinic acid, 3-methyl-2-oxovalerate, anserine, L-phenylalanine, L-tryptophan, N,N-Dimethylglycine, N6-acetyllysine, N-acetylysteine, propylene glycol, undecanoic acid, or xanthurenate. The altered level of the at least five metabolites can be a decreased level, and the at least five metabolites can be 1-methylhistidine, 2-aminomuconic acid semialdehyde, 2-methylguanosine, 2-oxoarginine, 2-pyrroloylglycine, 3-(3,5-diiodo-4-hydroxyphenyl)pyruvate, 3-dehydroxycarnitine, 3-hydroxyoctanoyl carnitine, 3-nitrotyrosine, 3′-O-methyladenosine, 4-guanidinobutanoic acid, 7-methylguanine, alanyl-serine, allantoic acid, argininic acid, arginyl-methionine, citric acid, creatine, cysteic acid, cysteinyl-cysteine, DL-2-aminooctanoic acid, dUMP, gamma-glutamyltyrosine, glutaminyl-lysine, glutarylcarnitine, glycerophosphocholine, glyceryl lactopalmitate, hippuric acid, IDP, L-acetylcarnitine, L-alanine, L-aspartyl-4-phosphate, L-carnitine, L-glutamine, L-histidine, L-isoleucine, L-phenylalanine, L-tyrosine, melatonin, N1-methyl-2-pyridone-5-carboxamide, N6,N6,N6-trimethyl-L-lysine, N-acetyl-L-methionine, nicotine imine, N-ribosylhistidine, 0-isobutyryl-L-carnitine, phenol sulphate, phenylpyruvic acid, picolinoylglycine, proline betaine, pyrogallol-2-O-glucuronide, riboflavin, serinyl-hydroxyproline, sphinganine 1-phosphate, trans-3-coumarate, tyrosyl-glutamate, urocanic acid, 1H-indole-3-carboxaldehyde, 2-hydroxyadenine, allodesmosine, arginyl-histidine, guanine, hypoxanthine, LysoPC(14:0), LysoPC(15:0), LysoPC(18:3(6Z,9Z,12Z)), LysoPE(0:0/18:1(11Z)), phosphoric acid, pyrrolidine, uridine, 1,9-dimethylurate, 2-(3-carboxy-3-aminopropyl)-L-histidine, adenosine diphosphate ribose, adenosine thiamine triphosphate, ADP, ATP, cholesterol glucuronide, D-galactose, D-glucose, GTP, malonyl-camitin, N-acetyl-D-glucosamine, NAD⁺, NADH, NADP⁺, N-decanoylglycine, phosphoribosyl-AMP, purine, or tiglylcarnitine. The detecting can include LC-MS, NMR, GC-MS, CE-MS, FTIR, or combinations thereof. The mammal can be a human. The sample can be whole blood, serum, plasma, urine, CSF, saliva, jejunum tissue, lung tissue, heart tissue, kidney tissue, skin tissue, bone marrow, gastrointestinal tract tissue, cardiovascular system tissue, CNS tissue, hematopoietic cells, or a fecal sample. The radiation exposure can occur during an event selected from the group consisting of an industrial accident, terrorist attack, military action, commercial exposure, medical procedure, environmental exposure, and any combinations thereof. The radiation exposure can include a radiation source selected from the group consisting of ionizing radiation, non-ionizing radiation, low energy particles, and high energy particles.

In another aspect, this document features methods for monitoring a mammal having been administered a radiation counter measure following radiation exposure. The methods can include, or consist essentially of, (a) detecting a presence or absence of at least one enriched metabolic pathway in a sample from a mammal having been administered a radiation counter measure following radiation exposure; (b) classifying the radiation counter measure as being an effective treatment for the mammal if the presence of the enriched metabolic pathways is not detected; and (c) administering the radiation counter measure to the mammal. The at least one enriched metabolic pathway can be Warburg effect, arginine and proline metabolism, glutamate metabolism, pyruvate metabolism, fatty acid metabolism, propanoate metabolism, nicotinate and nicotinamide metabolism, gluconeogenesis, ammonia recycling, pyrimidine metabolism, urea cycle, selenoamino acid metabolism, mitochondrial beta-oxidation of long chain saturated fatty acids, mitochondrial beta-oxidation of short chain saturated fatty acids, cysteine metabolism, oxidation of branched chain fatty acids, glycolysis, purine metabolism, carnitine synthesis, glutathione metabolism, lactose synthesis, beta oxidation of very long chain fatty acids, or valine, leucine and isoleucine degradation. The mammal can be a human. The sample can be whole blood, serum, plasma, urine, CSF, saliva, jejunum tissue, lung tissue, heart tissue, kidney tissue, skin tissue, bone marrow, gastrointestinal tract tissue, cardiovascular system tissue, CNS tissue, hematopoietic cells, or a fecal sample. The radiation exposure can occur during an event selected from the group consisting of an industrial accident, terrorist attack, military action, commercial exposure, medical procedure, environmental exposure, and any combinations thereof. The radiation exposure can include a radiation source selected from the group consisting of ionizing radiation, non-ionizing radiation, low energy particles, and high energy particles.

In another aspect, this document features methods for monitoring a mammal having been administered a first radiation counter measure following radiation exposure. The methods can include, or consist essentially of, (a) detecting a presence or absence of at least one enriched metabolic pathway in a sample from a mammal having been administered a first radiation counter measure following radiation exposure; (b) classifying the first radiation counter measure as not being an effective treatment for the mammal if the presence of the enriched metabolic pathways is detected; and (c) administering a second radiation counter measure to the mammal. The at least one enriched metabolic pathway can be Warburg effect, arginine and proline metabolism, glutamate metabolism, pyruvate metabolism, fatty acid metabolism, propanoate metabolism, nicotinate and nicotinamide metabolism, gluconeogenesis, ammonia recycling, pyrimidine metabolism, urea cycle, selenoamino acid metabolism, mitochondrial beta-oxidation of long chain saturated fatty acids, mitochondrial beta-oxidation of short chain saturated fatty acids, cysteine metabolism, oxidation of branched chain fatty acids, glycolysis, purine metabolism, carnitine synthesis, glutathione metabolism, lactose synthesis, beta oxidation of very long chain fatty acids, or valine, leucine and isoleucine degradation. The mammal can be a human. The sample can be whole blood, serum, plasma, urine, CSF, saliva, jejunum tissue, lung tissue, heart tissue, kidney tissue, skin tissue, bone marrow, gastrointestinal tract tissue, cardiovascular system tissue, CNS tissue, hematopoietic cells, or a fecal sample. The radiation exposure can occur during an event selected from the group consisting of an industrial accident, terrorist attack, military action, commercial exposure, medical procedure, environmental exposure, and any combinations thereof. The radiation exposure can include a radiation source selected from the group consisting of ionizing radiation, non-ionizing radiation, low energy particles, and high energy particles.

Unless otherwise defined, all technical and scientific terms used herein have the same meaning as commonly understood by one of ordinary skill in the art to which this invention pertains. Although methods and materials similar or equivalent to those described herein can be used to practice the invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

The details of one or more embodiments of the invention are set forth in the accompanying drawings and the description below. Other features, objects, and advantages of the invention will be apparent from the description and drawings, and from the claims.

### DESCRIPTION OF THE DRAWINGS

FIGS. 1A-1C show the experimental design of the three-cohort metabolomics study highlighting the combined use of LC-MS and NMR. (FIG. 1A) Overall goal of the study is to identify biomarkers of radiation exposure and to monitor radioprotection following amifostine treatment. (FIG. 1B) Cohort variables include sample number, radiation exposure, species, and biofluid time point collection. (FIG. 1C) Detailed description of the combined NMR and LC-MS metabolomics approach. The figure was generated using free medical images from Servier Medical Art (smart.servier.com/) under the Creative Commons License Attribution 3.0 Unported (CC BY 3.0).

FIGS. 2A-2D show 1D ¹H NMR time-dependent metabolomics analysis of mice serum samples following 14 Gy ⁶⁰Co-γ-radiation exposure. (FIG. 2A) NMR metabolic trajectories of 14 Gy (top) and Sham (bottom) groups calculated by PLS (one Predictive Component (PC), Unit Variance (UV), 7-fold Cross Validation) from a baseline norm (5H) and visualized across time (Q²). * indicates model p-values <0.05 (FIG. 2B) A p-value versus fold change volcano plot comparing NMR data 14 Gy D4 to 5H baseline (LIMMA—moderated T-test, Benjamini-Hochberg correction). (FIG. 2C) Heatmap of putative NMR metabolite IDs comparing 14 Gy 5H (light green) to 14 Gy D4 (dark green). (FIG. 2D) MetaboAnalyst pathway enrichment analysis of the NMR and LC-MS metabolites dysregulated due to radiation exposure. The top perturbed pathways are displayed by fold enrichment.

FIGS. 3A-3C show a univariate analysis of cohort 1. LC-MS time-dependent metabolomics analysis of cohort 1 following 14 Gy ⁶¹Co-γ-radiation exposure. (FIG. 3A) LC-MS metabolic trajectories of 14 Gy (top) and Sham (bottom) groups calculated by PLS (one Predictive Component (PC), Unit Variance (UV), 7-fold Cross Validation) from a baseline norm (5H) and visualized across time (Q²). * indicates models p-value <0.05 (FIG. 3B) Model analysis by SUS principles for significant effects as a result of radiation across two time points 14GyD4, 14GyD1 and a control ShD2 showing within and between group comparisons. (FIG. 3C) T v U scores plot (PLS—1 Predictive Component, Unit Variance, 7-fold Cross Validation) comparing 14 Gy D4 to 5H baseline NMR data (left) and LC-MS data (right) and p-value v fold change volcano plot (LIMMA—moderated T-test, Benjamini-Hochberg correction) comparing 14 Gy D4 to 5H baseline NMR data (left) and LC-MS data (right)

FIG. 4 shows a univariate analysis of cohort 1. Heatmap of LC-MS mice serum for 14Gy D4 (vs 5H).

FIGS. 5A-5D show LC-MS time-dependent metabolomics analysis of NHP serum samples following either 5.8 or 7.2 Gy ⁶¹Co-γ-radiation exposure. (FIG. 5A) LC-MS metabolic trajectories from NHP serum after exposure to 5.8 Gy (top) or 7.2 Gy (bottom) radiation dose. Metabolic trajectories were calculated by PLS (one Predictive Component (PC), Unit Variance (UV), 7-fold Cross Validation) from a baseline norm (5H) and visualized across time (Q²). * indicates model p-values <0.05 (FIG. 5B) A p-value versus fold change volcano plot comparing LC-MS data 7.2 Gy D1 to D4 (LIMMA—moderated T-test, Benjamini-Hochberg correction). (FIG. 5C) Heatmap of putative LC-MS metabolite IDs comparing 7.2 Gy D−7 (light purple) to 7.2 Gy D8 (dark purple). (FIG. 5D) MetaboAnalyst pathway enrichment analysis of the LC-MS metabolites dysregulated due to radiation exposure. The top perturbed pathways are displayed by fold enrichment.

FIGS. 6A-6D show LC-MS time-dependent metabolomics analysis of cohort 2 samples following either 5.8 or 7.2 Gy ⁶¹Co-γ-radiation exposure. (FIG. 6A) NMR metabolic trajectories from NHP serum after exposure to 5.8 Gy (top) or 7.2 Gy (bottom) radiation dose. Metabolic trajectories were calculated by PLS (one Predictive Component (PC), Unit Variance (UV), 7-fold Cross Validation) from a baseline norm (5H) and visualized across time (Q²). * indicates models p-value <0.05 (FIG. 6B) NMR model analysis by SUS principles for significant effects (VIP >1) as a result of radiation across within group 5.8 Gy D2 and 7.2 Gy 8H compared to respective controls and between group comparison of 5.8 Gy D2 and 7.2 Gy 8H. (FIG. 6C) LC-MS model analysis by SUS principles for significant effects (VIP >1) as a result of radiation across within group comparisons 5.8 Gy 8H, 7.2 Gy 8H, and 7.2 Gy D8 compared to respective controls and between group comparisons 5.8 Gy 8H vs 7.2 Gy 8H and 7.2 Gy 8H vs 7.2 Gy D8. (FIG. 6D) LC-MS T v U scores plot (PLS—1 Predictive Component, Unit Variance, 7-fold Cross Validation) comparing 7.2 Gy D8 to 7.2 Gy D−7 baseline LC-MS data.

FIGS. 7A-7E show combined ¹H NMR and LC-MS metabolomic analysis of cohort 3 exposed to 9.6 Gy ⁶¹Co γ-radiation. (FIG. 7A) 0 mg/kg (RAD) and (FIG. 7B) 200 mg/kg (RAD_Am200)—trajectories calculated by PLS (1 Predictive Component (PC), Unit Variance (UV), 7-fold Cross Validation), from a baseline norm (D−5), visualized across time (Q²). (FIG. 7C) LC-MS (left) and NMR (right) model analysis by SUS principles for significant effects (VIP >1) as a result of radiation for between group comparison RAD D1 vs D5 and RAD+200 mg/kg D5 vs D9 (FIG. 7D-FIG. 7E) LC-MS and NMR T v U scores plot (PLS—1 Predictive Component, Unit Variance, 7-fold Cross Validation) and p-value v FC volcano plot (LIMMA—moderated T-test, Benjamini-Hochberg correction) for 9.6 Gy radiation exposure (RAD D1 vs D5) and 9.6 Gy radiation exposure with amifostine treatment at 200 mg/kg (RAD+Am200 D5 vs D9).

FIGS. 8A-8D shows combined ¹H NMR and LC-MS metabolomic analysis of mice whole blood exposed to 9.6 Gy 60Co-γ-radiation. (FIG. 8A) Heatmap of putative LC-MS metabolites comparing 9.6 Gy D1 (light blue) to D5 (gray) and (FIG. 8B) MetaboAnalyst pathway enrichment analysis of NMR and LC-MS metabolites from (A). Top perturbed pathways are displayed by fold enrichment. (FIG. 8C) Heatmap of putative LC-MS metabolites comparing 9.6 Gy and 200 mg/kg amifostine treatment D5 (gray) to D9 (navy blue) and (FIG. 8D) MetaboAnalyst pathway enrichment analysis of NMR and LC-MS metabolites from (FIG. 8C). Top perturbed pathways are displayed by fold enrichment.

FIGS. 9A-9B show (FIG. 9A) Heatmap of 9.6 Gy D1 (light blue) vs D5 (gray) NMR putative metabolite IDs. (FIG. 9B) Heatmap of 9.6 Gy and 200 mg/kg amifostine treatment D5 (gray) vs D9 (navy blue) NMR putative metabolite IDs.

FIG. 10 shows NMR and MS metabolomics trajectories induced by cohort 3 treatment with amifostine that is independent of radiation exposure. Values were calculated by PLS (1 Predictive Component (PC), Unit Variance (UV), 7-fold Cross Validation), from a baseline norm (D−5), visualized across time (Q²)—5H, D1, D5, D9.

FIGS. 11A-11D show (FIG. 11A) a Venn diagram summarizing the metabolic pathways from the three cohorts that were dysregulated due to radiation exposure. (FIG. 11B) Venn diagram summarizing the metabolic pathways from cohort three that were dysregulated with or without amifostine treatment (200 mg/kg). (FIG. 11C) Representative metabolite trajectories from the LC-MS (triangles) and NMR (circles) data sets within cohort one or two. The metabolite trajectories are labeled with the associated dysregulated metabolic pathway and track the response to radiation exposure. * denotes p-values <0.05 and VIP >1. (FIG. 11D) Representative metabolite trajectories from the LC-MS (triangles) and NMR (circles) data sets within cohort three. The trajectories compare a metabolic response to radiation exposure (RAD D1 to D5, gray) and the metabolic response to a radiation exposure and a 200 mg/kg amifostine pretreatment (RAD+200 D5 to D9, dark blue). The metabolite trajectories are labeled with the associated dysregulated metabolic pathway and track the metabolic response to amifostine pretreatment. * denotes p-values <0.05 and VIP >1.

FIGS. 12A-12C show receiver operative characteristic (ROC) analysis of the three cohorts (FIG. 12A, FIG. 12B, and FIG. 12C, respectively) exposure to radiation 5 features provides an accuracy between 0.866-0.996.

### DETAILED DESCRIPTION

This document relates to methods and materials for assessing radiation exposure. For example, methods and materials provided herein can be used to determine whether a mammal (e.g., a human) has been exposed to radiation and/or whether a mammal (e.g., a human) is at risk of developing one or more radiation injuries (e.g., CRI and/or ARS) following radiation exposure (e.g., radiation therapy administered as a part of a cancer treatment). In some cases, a mammal (e.g., a human) can be assessed to determine whether it has been exposed to radiation and/or whether it is likely to develop one or more radiation injuries (e.g., CRI and/or ARS) following radiation exposure (e.g., radiation therapy administered as a part of a cancer treatment) by detecting the presence or absence of an altered level (e.g., an increased level or a decreased level) of one or more metabolites in a sample obtained from the mammal. As described herein, a panel that includes at least five (e.g., five, six, seven, eight, nine, ten, eleven, or more) of the following metabolites can be used to determine whether a mammal (e.g., a human) has been exposed to radiation and/or is likely to develop one or more radiation injuries (e.g., CRI and/or ARS) following radiation exposure (e.g., radiation therapy administered as a part of a cancer treatment): 12-oxo-20-trihydroxy-leukotriene B4, 1H-indole-3-carboxaldehyde, 2-amino-3-phosphonopropionic acid, 3-hydroxybutyrate, 3-methylcrotonyl-CoA, 5-hydroxyindoleacetic acid, ADP, alanyl-alanine, androstanediol, anserine, argininic acid, ATP, celastrol, cortisol, cysteic acid, deoxyguanosine, dihydroneopterin phosphate, dUMP, gamma-glutamylthreonine, ganglioside GA1 (d18:1/26:0), ganglioside GM3 (d18:0/26:1(17Z)), guanine, hypoxanthine, L-aspartyl-4-phosphate, L-carnitine, L-threo-3-phenylserine, lysoPC(14:0), lysoPC(15:0), NAD⁺, NADP⁺, nicotine imine, pantothenic acid, phenylalanine, phosphoric acid, pimelylcarnitine, S-methylmethionine, tiglylcarnitine, tiglyl-CoA, trans-3-coumarate, UDP-D-galactose, uracil, uridine, valine, and vanylglycol. For example, an increased level of one or more (e.g., one, two, three, four, five, six, seven, or more) metabolites (e.g., 12-oxo-20-trihydroxy-leukotriene B4, 2-amino-3-phosphonopropionic acid, 3-hydroxybutyrate, 3-methylcrotonyl-CoA, 5-hydroxyindoleacetic acid, anserine, deoxyguanosine, gamma-glutamylthreonine, ganglioside GA1 (d18:1/26:0), ganglioside GM3 (d18:0/26:1(17Z)), L-aspartyl-4-phosphate, L-carnitine, pantothenic acid, phenylalanine, S-methylmethionine, tiglyl-CoA, UDP-D-galactose, uridine, valine, and vanylglycol) and/or a decreased level of one or more (e.g., one, two, three, four, five, six, seven, or more) metabolites (e.g., 1H-indole-3-carboxaldehyde, ADP, alanyl-alanine, androstanediol, argininic acid, ATP, celastrol, cortisol, cysteic acid, dihydroneopterin phosphate, dUMP, guanine, hypoxanthine, L-threo-3-phenylserine, lysoPC(14:0), lysoPC(15:0), NAD⁺, NADP⁺, nicotine imine, phosphoric acid, pimelylcarnitine, tiglylcarnitine, trans-3-coumarate, and uracil) can be present in a sample obtained from a mammal (e.g., a human) that has been exposed to radiation and/or is likely to develop one or more radiation injuries (e.g., CRI and/or ARS) following radiation exposure (e.g., radiation therapy administered as a part of a cancer treatment). In some cases, a mammal (e.g., a human) can be identified as having been exposed to radiation and/or as being at risk of developing one or more radiation injuries (e.g., CRI and/or ARS) following radiation exposure (e.g., radiation therapy administered as a part of a cancer treatment) based, at least in part, on the presence of an altered level (e.g., an increased level or a decreased level) of at least five metabolites in a sample obtained from the mammal.

In some cases, a mammal (e.g., a human) can be assessed to determine whether it has been exposed to radiation and/or whether it is likely to develop one or more radiation injuries (e.g., CRI and/or ARS) following radiation exposure (e.g., radiation therapy administered as a part of a cancer treatment) by detecting the presence or absence of one or more enriched metabolic pathways in a sample obtained from the mammal. As described herein, a panel that includes enrichment of at least one (e.g., one, two, three, four, five, six, seven eight, or more) of the following metabolic pathways can be used to determine whether a mammal (e.g., a human) has been exposed to radiation and/or is likely to develop one or more radiation injuries (e.g., CRI and/or ARS) following radiation exposure: ammonia recycling, androstenedione metabolism, arginine and proline metabolism, betaine metabolism, beta oxidation of very long chain fatty acids, bile acid biosynthesis, carnitine synthesis, biotin metabolism, cysteine metabolism, butyrate metabolism, fatty acid metabolism, caffeine metabolism, gluconeogenesis, cardiolipin biosynthesis, glutamate metabolism, catecholamine biosynthesis, glutathione metabolism, D-arginine and D-ornithine metabolism, glycolysis, de novo triacylglycerol biosynthesis, lactose synthesis, degradation of superoxides, mitochondrial beta-oxidation of long chain saturated fatty acids, estrone metabolism, mitochondrial beta-oxidation of short chain saturated fatty acids, ethanol degradation, nicotinate and nicotinamide metabolism, fatty acid biosynthesis, oxidation of branched chain fatty acids, fatty acid elongation in mitochondria, propanoate metabolism, folate metabolism, purine metabolism, glycerolipid metabolism, pyrimidine metabolism, ketone body metabolism, pyruvate metabolism, lysine degradation, selenoamino acid metabolism, malate-aspartate shuttle, urea cycle, methionine metabolism, valine, leucine and isoleucine degradation, methylhistidine metabolism, Warburg effect, mitochondrial beta-oxidation of medium chain saturated fatty acids, alanine metabolism, mitochondrial electron transport chain, amino sugar metabolism, nucleotide sugars metabolism, arachidonic acid metabolism, pantothenate and CoA biosynthesis, aspartate metabolism, pentose phosphate pathway, beta-alanine metabolism, phosphatidylcholine biosynthesis, citric acid cycle, phosphatidylethanolamine biosynthesis, fructose and mannose degradation, phosphatidylinositol phosphate metabolism, galactose metabolism, phospholipid biosynthesis, glucose-alanine cycle, phytanic acid peroxisomal oxidation, glycerol phosphate shuttle, plasmalogen synthesis, glycine and serine metabolism, porphyrin metabolism, histidine metabolism, pyruvaldehyde degradation, inositol metabolism, spermidine and spermine biosynthesis, inositol phosphate metabolism, starch and sucrose metabolism, lactose degradation, steroid biosynthesis, phenylacetate metabolism, steroidogenesis, phenylalanine and tyrosine metabolism, sulfate/sulfite metabolism, pterine biosynthesis, taurine and hypotaurine metabolism, retinol metabolism, thiamine metabolism, riboflavin metabolism, threonine and 2-oxobutanoate degradation, sphingolipid metabolism, thyroid hormone synthesis, transfer of acetyl groups into mitochondria, trehalose degradation, tryptophan metabolism, ubiquinone biosynthesis, tyrosine metabolism, vitamin B6 metabolism, androgen and estrogen metabolism, and vitamin K metabolism. For example, enrichment of one or more (e.g., one, two, three, four, five, six, seven, or more) metabolic pathways (e.g., Warburg effect, arginine and proline metabolism, glutamate metabolism, pyruvate metabolism, fatty acid metabolism, propanoate metabolism, nicotinate and nicotinamide metabolism, gluconeogenesis, ammonia recycling, pyrimidine metabolism, urea cycle, selenoamino acid metabolism, mitochondrial beta-oxidation of long chain saturated fatty acids, mitochondrial beta-oxidation of short chain saturated fatty acids, cysteine metabolism, oxidation of branched chain fatty acids, glycolysis, purine metabolism, carnitine synthesis, glutathione metabolism, lactose synthesis, beta oxidation of very long chain fatty acids, and valine, leucine and isoleucine degradation) can be present in a sample obtained from a mammal (e.g., a human) that has been exposed to radiation and/or is likely to develop one or more radiation injuries (e.g., CRI and/or ARS) following radiation exposure. In some cases, a mammal (e.g., a human) can be identified as having been exposed to radiation and/or as being at risk of developing one or more radiation injuries (e.g., CRI and/or ARS) following radiation exposure based, at least in part, on the presence of at least one enriched metabolic pathway in a sample obtained from the mammal.

In some cases, this document provides methods and materials for treating a mammal (e.g., a mammal identified as having been exposed to radiation and/or as being likely to develop one or more radiation injuries following radiation exposure). For example, one or more radiation counter measures (e.g., radioprotective agents, radio-mitigation agents, and/or radio-therapeutic agents) can be administered to a mammal (e.g., a human) identified as having been exposed to radiation and/or as being likely to develop one or more radiation injuries following radiation exposure as described herein (e.g., based, at least in part, on the presence of an altered level of at least five metabolites in a sample from the mammal and/or the presence of at least one enriched metabolic pathway) to treat that mammal.

Any appropriate mammal can be assessed and/or treated as described herein. Examples of mammals that can be assessed and/or treated as described herein include, without limitation, humans, non-human primates (e.g., monkeys), dogs, cats, horses, cows, pigs, sheep, mice, and rats. In some cases, a mammal can have cancer. In some cases, a human (e.g., a human having cancer) can be assessed to determine whether or not that mammal is likely to develop one or more radiation injuries (e.g., CRI and/or ARS) following radiation exposure (e.g., radiation therapy administered as a part of a cancer treatment).

When assessing and/or treating a mammal (e.g., a human) having cancer as described herein, the cancer can be any type of cancer. A cancer can be any stage of cancer (e.g., stage I, stage II, stage III, or stage IV). A cancer can be any grade of cancer (e.g., grade 1, grade 2, or grade 3). In some cases, a cancer can be a primary cancer (e.g., a localized primary cancer). In some cases, a cancer can have metastasized. In some cases, the methods described herein can include identifying a mammal (e.g., a human) as having cancer. Any appropriate method can be used to identify a mammal as having cancer. For example, physical examination (e.g., a pelvic examination), imaging techniques (e.g., transvaginal ultrasound, hysteroscopy, X-ray, computerized tomography (CT) scanning, and positron emission tomography (PET) scanning), and biopsy techniques can be used to identify a mammal (e.g., a human) as having cancer.

Radiation exposure can be associated with any event. Examples of events that can result in radiation exposure include, without limitation, radiation therapy (e.g., radiation therapy administered as a part of a cancer treatment), industrial exposure (e.g., chronic industrial exposure and industrial accidents such as an accident at a nuclear industrial facility), military actions, commercial exposure, medical procedures, and environmental exposure (e.g., high-altitude exposure).

Radiation exposure can include any type(s) of radiation. In some cases, radiation exposure can include ionizing radiation. In some cases, radiation exposure can include non-ionizing radiation. In some cases, radiation exposure can include low energy particles. In some cases, radiation exposure can include high energy particles (e.g., neutrons, electrons, and positrons). Examples of types of radiation that can be included in a radiation exposure include, without limitation, alpha rays, beta rays, neutrons, gamma rays, and X-rays.

Radiation exposure can include radiation emitted from any source. In some cases, radiation can be from a natural source (e.g., can be emitted from a natural source). In some cases, radiation can be from an artificial source (e.g., can be artificially produced and emitted from a man-made source). Examples of sources of radiation include, without limitation, radioactive substances (e.g., radium and radioisotopes such as potassium-40 and carbon-14), X-ray machines, nuclear reactors, particle accelerators (e.g., linear particle accelerators), nuclear weapons, and nuclear and/or medical waste.

Radiation exposure can be to any part of the body of the mammal. In some cases, radiation exposure can be whole-body radiation exposure. In some cases, radiation exposure can be partial-body (e.g., skin, GI tract, and bone marrow) exposure. In some cases, radiation exposure can be internal exposure (e.g., from ingestion and/or inhalation). In some cases, radiation exposure can be external exposure.

A radiation injury can be any type of radiation injury. A radiation injury can be any grade of radiation injury (e.g., grade 1, grade 2, or grade 3). Examples of radiation injuries include, without limitation, CRI, hematopoietic-ARS (H-ARS), gastrointestinal (GI) ARS (GI-ARS), and neurovascular syndrome.

The presence or absence of an altered level (e.g., an increased level or a decreased level) of any number of metabolites in a sample obtained from a mammal can be used to determine whether or not that mammal has been exposed to radiation and/or whether or not that mammal is likely to develop one or more radiation injuries (e.g., CRI and/or ARS) following radiation exposure (e.g., radiation therapy administered as a part of a cancer treatment). In some cases, the presence or absence of an altered level (e.g., an increased level or a decreased level) of five or more (e.g., five, six, seven, eight, nine, ten, eleven, or more) in a sample obtained from a mammal can be used to determine whether or not that mammal has been exposed to radiation and/or whether or note that mammal is likely to develop one or more radiation injuries (e.g., CRI and/or ARS) following radiation exposure. In some cases, the presence or absence of an altered level (e.g., an increased level or a decreased level) of from about 5 metabolites to about 200 metabolites (e.g., from about 5 to about 180, from about 5 to about 160, from about 5 to about 200, from about 5 to about 150, from about 5 to about 140, from about 5 to about 125, from about 5 to about 100, from about 5 to about 75, from about 5 to about 50, from about 5 to about 40, from about 5 to about 30, from about 5 to about 20, from about 10 to about 200, from about 25 to about 200, from about 50 to about 200, from about 75 to about 200, from about 100 to about 200, from about 125 to about 200, from about 150 to about 200, from about 175 to about 200, from about 10 to about 180, from about 30 to about 150, from about 50 to about 125, from about 75 to about 100, from about 5 to about 50, from about 50 to about 100, or from about 100 to about 150 metabolites) in a sample obtained from a mammal can be used to determine whether or not that mammal is likely to develop one or more radiation injuries (e.g., CRI and/or ARS) following radiation exposure.

The presence or absence of an altered level (e.g., an increased level or a decreased level) of any appropriate metabolite in a sample obtained from a mammal can be used to determine whether or not that mammal been exposed to radiation and/or whether or not that mammal is likely to develop one or more radiation injuries (e.g., CRI and/or ARS) following radiation exposure (e.g., radiation therapy administered as a part of a cancer treatment). A metabolite can be part of any appropriate metabolic pathway (e.g., glucose metabolism pathways, de-novo lipid synthesis pathways, amino acid metabolism pathways (e.g., tryptophan metabolism pathways), the pathways described in Example 1, the pathways described in Table 3, the pathways described in Table 5, and the pathways described in Table 7). In some cases, a metabolite can be an amino acid (e.g., a branched chain amino acid). In some cases, a metabolite can be a lipid. In some cases, a metabolite can be a carbohydrate (e.g., a saccharide such as a monosaccharide). Examples of metabolites that for which the presence or absence of an altered level (e.g., an increased level or a decreased level) of the metabolite in a sample obtained from a mammal can be used to determine whether or not that mammal is likely to develop one or more radiation injuries (e.g., CRI and/or ARS) following radiation exposure (e.g., radiation therapy administered as a part of a cancer treatment) include, without limitation, 12-oxo-20-trihydroxy-leukotriene B4, 1H-indole-3-carboxaldehyde, 2-amino-3-phosphonopropionic acid, 3-hydroxybutyrate, 3-methylcrotonyl-CoA, 5-hydroxyindoleacetic acid, ADP, alanyl-alanine, androstanediol, anserine, argininic acid, ATP, celastrol, cortisol, cysteic acid, deoxyguanosine, dihydroneopterin phosphate, dUMP, gamma-glutamylthreonine, ganglioside GA1 (d18:1/26:0), ganglioside GM3 (d18:0/26:1(17Z)), guanine, hypoxanthine, L-aspartyl-4-phosphate, L-carnitine, L-threo-3-phenylserine, lysoPC(14:0), lysoPC(15:0), NAD⁺, NADP⁺, nicotine imine, pantothenic acid, phenylalanine, phosphoric acid, pimelylcarnitine, S-methylmethionine, tiglylcarnitine, tiglyl-CoA, trans-3-coumarate, UDP-D-galactose, uracil, uridine, valine, and vanylglycol.

The presence or absence of an enriched metabolic pathway in a sample obtained from a mammal can be used to determine whether or not that mammal has been exposed to radiation and/or whether or not that mammal is likely to develop one or more radiation injuries (e.g., CRI and/or ARS) following radiation exposure (e.g., radiation therapy administered as a part of a cancer treatment). In some cases, the presence or absence of one or more (e.g., one, two, three, four, five, six, seven, or more) enriched metabolic pathways in a sample obtained from a mammal can be used to determine whether or not that mammal has been exposed to radiation and/or whether or note that mammal is likely to develop one or more radiation injuries (e.g., CRI and/or ARS) following radiation exposure. In some cases, the presence or absence of from about one enriched metabolic pathway to about 100 enriched metabolic pathways (e.g., from about 1 to about 100, from about 1 to about 90, from about 1 to about 80, from about 1 to about 70, from about 1 to about 60, from about 1 to about 50, from about 1 to about 40, from about 1 to about 30, from about 1 to about 20, from about 1 to about 10, from about 10 to about 100, from about 20 to about 100, from about 30 to about 100, from about 40 to about 100, from about 50 to about 100, from about 60 to about 100, from about 70 to about 100, from about 80 to about 100, from about 90 to about 100, from about 10 to about 90, from about 20 to about 80, from about 30 to about 70, from about 40 to about 60, from about 10 to about 20, from about 20 to about 30, from about 30 to about 40, from about 40 to about 50, from about 50 to about 60, from about 60 to about 70, from about 70 to about 80, or from about 80 to about 90 enriched metabolic pathways) in a sample obtained from a mammal can be used to determine whether or not that mammal is likely to develop one or more radiation injuries (e.g., CRI and/or ARS) following radiation exposure. For example, the presence or absence of about 20 to about 25 metabolic pathways in a sample obtained from a mammal can be used to determine whether or not that mammal is likely to develop one or more radiation injuries (e.g., CRI and/or ARS) following radiation exposure.

The presence or absence of enrichment of any appropriate metabolic pathways in a sample obtained from a mammal can be used to determine whether or not that mammal been exposed to radiation and/or whether or not that mammal is likely to develop one or more radiation injuries (e.g., CRI and/or ARS) following radiation exposure (e.g., radiation therapy administered as a part of a cancer treatment). In some cases, a metabolic pathway can be a carbohydrate metabolism pathway. In some cases, a metabolic pathway can be a lipid synthesis pathway. In some cases, a metabolic pathway can be an amino acid metabolism pathway. In some cases, a metabolic pathway can be as described in Example 1. In some cases, a metabolic pathway can be as described in Table 3, Table 5, Table 7, and/or Table 9. Examples of metabolic pathways for which the presence or absence of enrichment of the metabolic pathway in a sample obtained from a mammal can be used to determine whether or not that mammal is likely to develop one or more radiation injuries (e.g., CRI and/or ARS) following radiation exposure (e.g., radiation therapy administered as a part of a cancer treatment) include, without limitation, Warburg effect, arginine and proline metabolism, glutamate metabolism, pyruvate metabolism, fatty acid metabolism, propanoate metabolism, nicotinate and nicotinamide metabolism, gluconeogenesis, ammonia recycling, pyrimidine metabolism, urea cycle, selenoamino acid metabolism, mitochondrial beta-oxidation of long chain saturated fatty acids, mitochondrial beta-oxidation of short chain saturated fatty acids, cysteine metabolism, oxidation of branched chain fatty acids, glycolysis, purine metabolism, carnitine synthesis, glutathione metabolism, lactose synthesis, beta oxidation of very long chain fatty acids, and valine, leucine and isoleucine degradation.

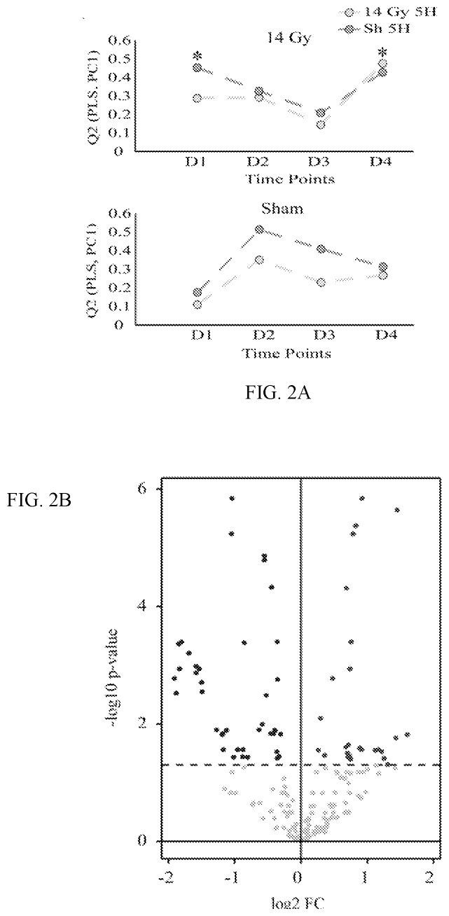

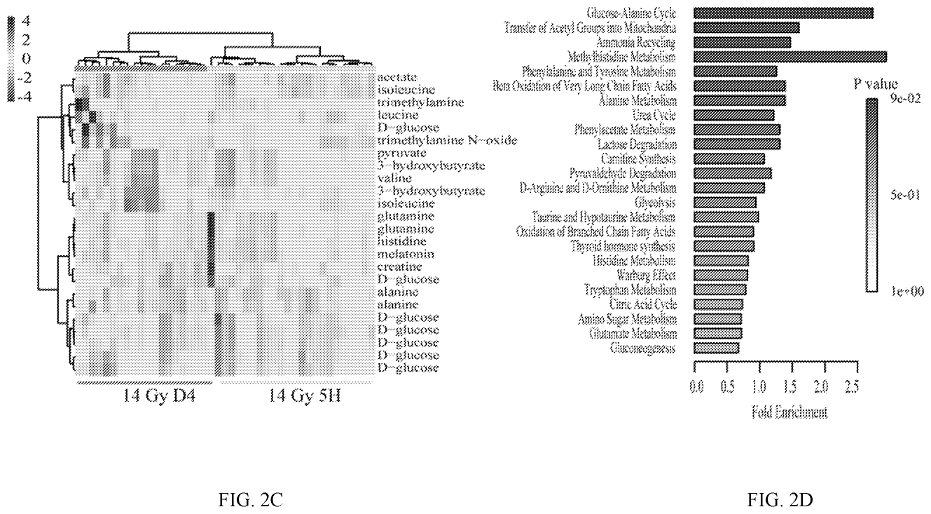

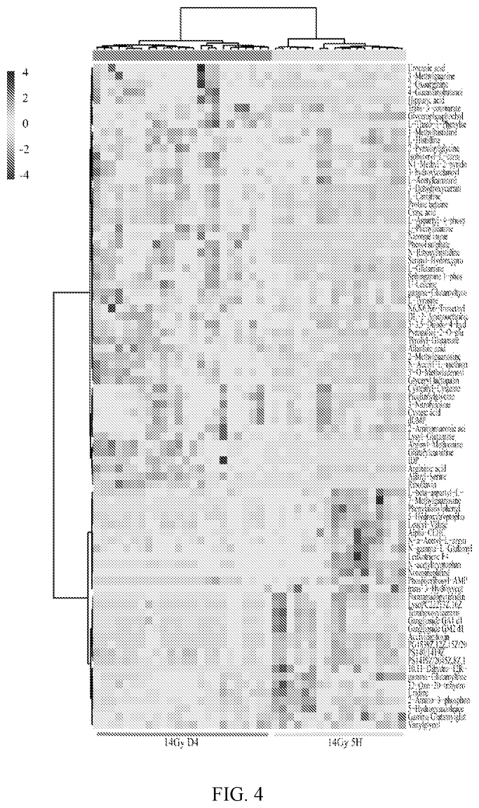

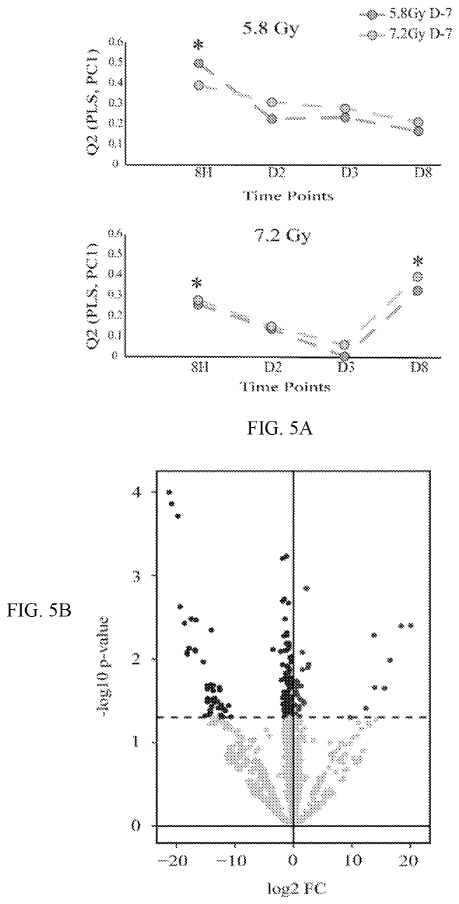

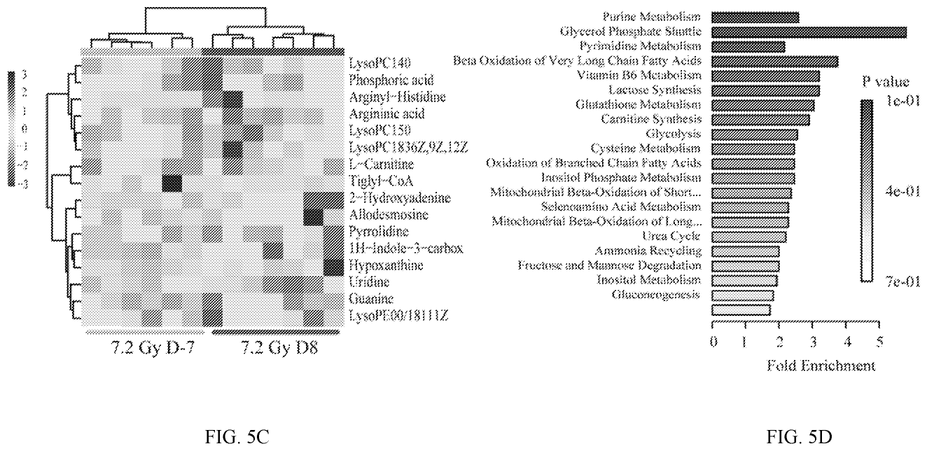

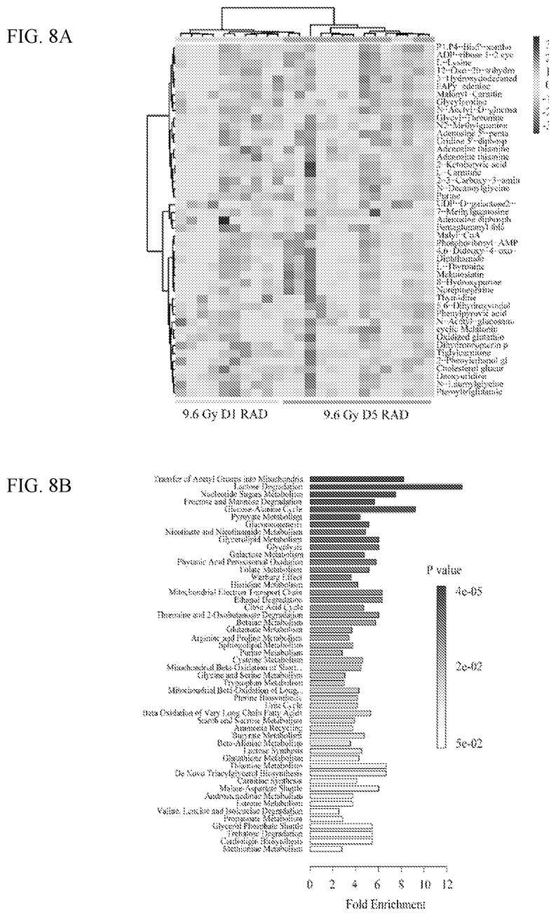

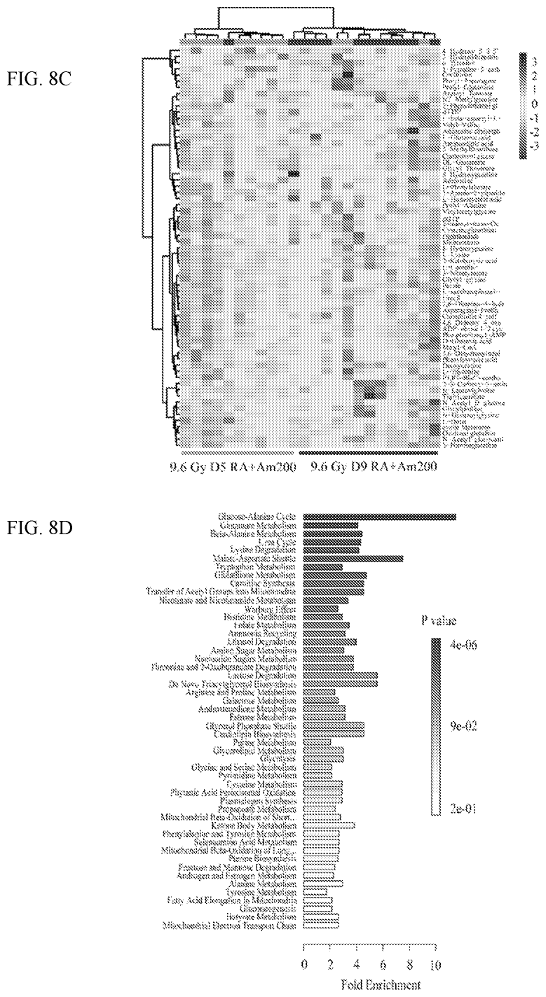

In some cases, a mammal (e.g., a human) can be identified as having been exposed to radiation and/or can be identified as likely to develop one or more radiation injuries (e.g., CRI and/or ARS) following radiation exposure (e.g., radiation therapy administered as a part of a cancer treatment) based, at least in part, on the presence of an increased level of five or more (e.g., five, six, seven, eight, nine, ten, eleven, or more) metabolites and/or the presence of one or more (e.g., one, two, three, four, five, six, seven, or more) enriched metabolic pathways in a sample obtained from the mammal. The term “increased level” as used herein with respect to a level of a metabolite refers to any level that is greater than a reference level of that metabolite. The term “enriched” as used herein with respect to a metabolic pathway refers to a metabolic pathway having an increased level of any component (e.g., a metabolite) of that metabolic pathway. The term “reference level” as used herein with respect to a metabolite refers to the level of that metabolite or that metabolic pathway typically observed in a sample (e.g., a control sample) from one or more comparable mammals (e.g., humans of comparable age) that have been exposed to radiation and do not have one or more radiation injuries. Control samples can include, without limitation, samples from mammals that have not been exposed to radiation, cells lines that have not been exposed to radiation, and samples from animal models (e.g., mice or non-human primates) that have not been exposed to radiation. Examples of metabolites that can have increased levels in a sample from a mammal that has been exposed to radiation and/or is likely to develop one or more radiation injuries (e.g., CRI and/or ARS) following radiation exposure (e.g., radiation therapy administered as a part of a cancer treatment) include, without limitation, 10,11-dihydro-12r-hydroxy-leukotriene E4, 12-oxo-20-trihydroxy-leukotriene B4, 2-amino-3-phosphonopropionic acid, 3-hydroxybutyrate, 5-hydroxyindoleacetic acid, 5-hydroxytryptophol glucuronide, 7-methylguanosine, acetyldigitoxin, alpha-CEHC, D-glucose, formamidopyrimidine nucleoside triphosphate, gamma glutamylglutamic acid, gamma-glutamylthreonine, ganglioside GA1 (d18:1/16:0), ganglioside GM2 (d18:1/18:1(11z)), L-beta-aspartyl-L-threonine, leucyl-valine, leukotriene F4, L-isoleucine, L-leucine, lysoPC(22:2(13z,16z)), N-a-acetyl-L-arginine, N-acetyltryptophan, N-gamma-L-glutamyl-D-alanine, PG(18:3(9Z,12Z,15Z)/20:3(5Z,8Z,11Z)), phenylalanylphenylalanine, phosphoribosyl-AMP, p-hydroxyphenylacetic acid, PS(14:0/14:1(9Z)), PS(20:4(5Z,8Z,11Z,14Z)/14:1(9Z)), pyruvate, tetrahexosylceramide (d18:1/16:0), trans-3-hydroxycotinine glucuronide, trimethylamine N-oxide, uridine, valine, vanylglycol, L-carnitine, tiglyl-CoA, 2,2-dimethylsuccinic acid, 3-methyl-2-oxovalerate, anserine, L-phenylalanine, L-tryptophan, N,N-Dimethylglycine, N6-acetyllysine, N-acetylysteine, propylene glycol, undecanoic acid, and xanthurenate. In some cases, an increased level of a metabolite can be a level that is at least 1 (e.g., at least 2, at least 3, at least 5, at least 8, at least 10, at least 15, or at least 20) fold greater relative to a reference level of that metabolite. In some cases, when control samples have an undetectable level of a metabolite, an increased level can be any detectable level of that metabolite. It will be appreciated that levels from comparable samples are used when determining whether or not a particular level is an increased level. In some cases, a metabolite that can have an increased level and/or a metabolic pathway that can be enriched in a sample from a mammal that has been exposed to radiation and/or is likely to develop one or more radiation injuries (e.g., CRI and/or ARS) following radiation exposure can be as described in Example 1. For example, a metabolite that can have an increased level in a sample from a mammal that has been exposed to radiation and/or is likely to develop one or more radiation injuries (e.g., CRI and/or ARS) following radiation exposure can be as described in Table 2, Table 4, Table 6, and/or Table 8. For example, a metabolite that can have an increased level in a sample from a mammal that has been exposed to radiation and/or is likely to develop one or more radiation injuries (e.g., CRI and/or ARS) following radiation exposure can be as shown in FIG. 2, FIG. 4, and/or FIG. 5. For example, a metabolic pathway that can be enriched in a sample from a mammal that has been exposed to radiation and/or is likely to develop one or more radiation injuries (e.g., CRI and/or ARS) following radiation exposure can be as described in Table 3, Table 5, Table 7, and/or Table 9. For example, a metabolic pathway that can be enriched in a sample from a mammal that has been exposed to radiation and/or is likely to develop one or more radiation injuries (e.g., CRI and/or ARS) following radiation exposure can be as shown in FIG. 2, FIG. 5, and/or FIG. 8.

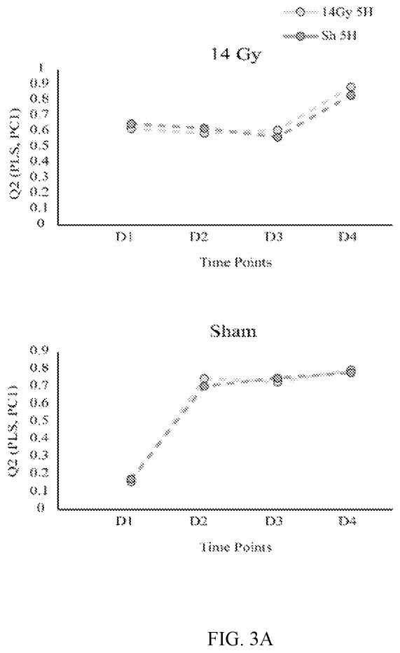

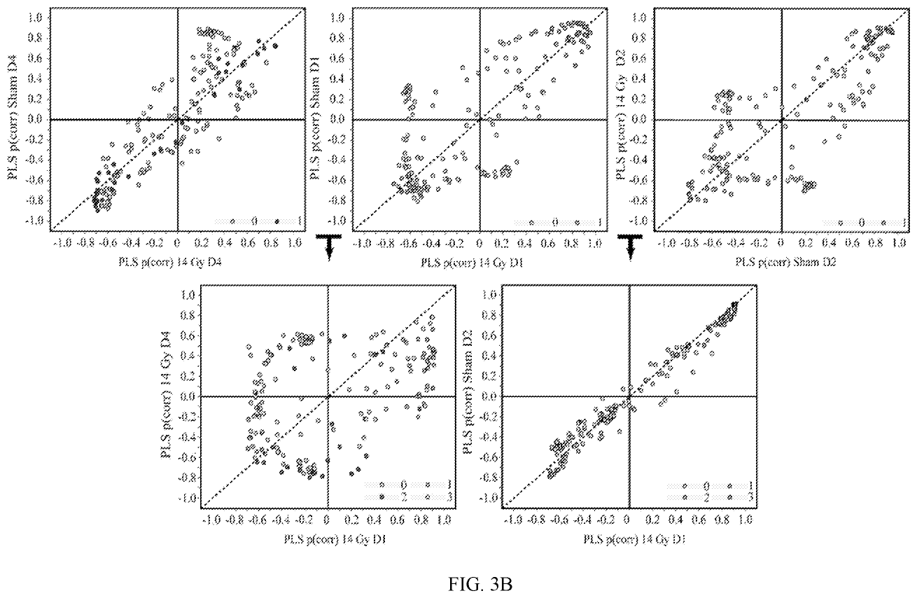

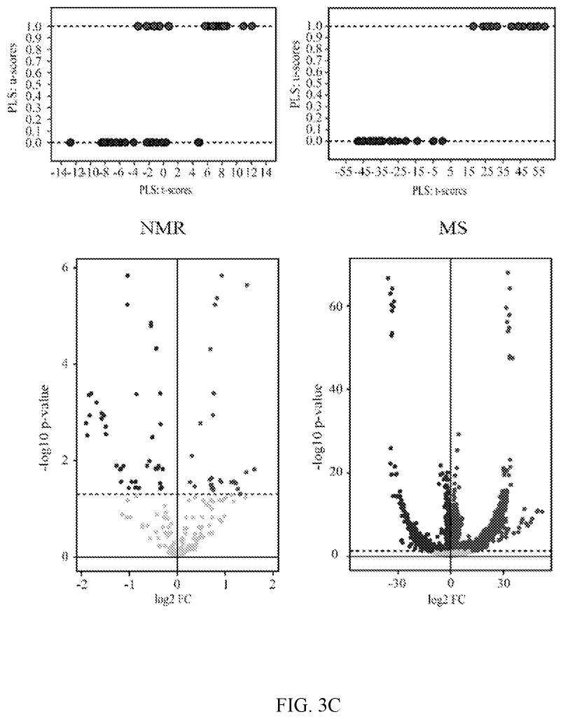

In some cases, a mammal (e.g., a human) can be identified as having been exposed to radiation and/or can be identified as likely to develop one or more radiation injuries (e.g., CRI and/or ARS) following radiation exposure (e.g., radiation therapy administered as a part of a cancer treatment) based, at least in part, on the presence of a decreased level of five or more (e.g., five, six, seven, eight, nine, ten, eleven, or more) metabolites in a sample obtained from the mammal. The term “decreased level” as used herein with respect to a level of a metabolites refers to any level that is less than a reference level of that metabolite. The term “reference level” as used herein with respect to a metabolite refers to the level of that metabolite typically observed in a sample (e.g., a control sample) from one or more comparable mammals (e.g., humans of comparable age) that have been exposed to radiation and do not have one or more radiation injuries. Control samples can include, without limitation, samples from mammals that have not been exposed to radiation, cells lines that have not been exposed to radiation, and samples from animal models (e.g., mice or non-human primates) that have not been exposed to radiation. Examples of metabolites that can have decreased levels in a sample from a mammal that has been exposed to radiation and/or is likely to develop one or more radiation injuries (e.g., CRI and/or ARS) following radiation exposure (e.g., radiation therapy administered as a part of a cancer treatment) include, without limitation, 1-methylhistidine, 2-aminomuconic acid semialdehyde, 2-methylguanosine, 2-oxoarginine, 2-pyrroloylglycine, 3-(3,5-diiodo-4-hydroxyphenyl)pyruvate, 3-dehydroxycarnitine, 3-hydroxyoctanoyl carnitine, 3-nitrotyrosine, 3′-O-methyladenosine, 4-guanidinobutanoic acid, 7-methylguanine, alanyl-serine, allantoic acid, argininic acid, arginyl-methionine, citric acid, creatine, cysteic acid, cysteinyl-cysteine, DL-2-aminooctanoic acid, dUMP, gamma-glutamyltyrosine, glutaminyl-lysine, glutarylcarnitine, glycerophosphocholine, glyceryl lactopalmitate, hippuric acid, IDP, L-acetylcarnitine, L-alanine, L-aspartyl-4-phosphate, L-carnitine, L-glutamine, L-histidine, L-isoleucine, L-phenylalanine, L-tyrosine, melatonin, N1-methyl-2-pyridone-5-carboxamide, N6,N6,N6-trimethyl-L-lysine, N-acetyl-L-methionine, nicotine imine, N-ribosylhistidine, O-isobutyryl-L-carnitine, phenol sulphate, phenylpyruvic acid, picolinoylglycine, proline betaine, pyrogallol-2-O-glucuronide, riboflavin, serinyl-hydroxyproline, sphinganine 1-phosphate, trans-3-coumarate, tyrosyl-glutamate, urocanic acid, 1H-indole-3-carboxaldehyde, 2-hydroxyadenine, allodesmosine, arginyl-histidine, guanine, hypoxanthine, LysoPC(14:0), LysoPC(15:0), LysoPC(18:3(6Z,9Z,12Z)), LysoPE(0:0/18:1(11Z)), phosphoric acid, pyrrolidine, uridine, 1,9-dimethylurate, 2-(3-carboxy-3-aminopropyl)-L-histidine, adenosine diphosphate ribose, adenosine thiamine triphosphate, ADP, ATP, cholesterol glucuronide, D-galactose, D-glucose, GTP, malonyl-camitin, N-acetyl-D-glucosamine, NAD⁺, NADH, NADP⁺, N-decanoylglycine, phosphoribosyl-AMP, purine, and tiglylcarnitine. In some cases, an increased level of a metabolite can be a level that is at least 1 (e.g., at least 2, at least 3, at least 5, at least 8, at least 10, at least 15, or at least 20) fold less relative to a reference level of that metabolite. It will be appreciated that levels from comparable samples are used when determining whether or not a particular level is a decreased level. In some cases, a metabolite that can have decreased level in a sample from a mammal that has been exposed to radiation and/or is likely to develop one or more radiation injuries (e.g., CRI and/or ARS) following radiation exposure can be as described in Example 1. For example, a metabolite that can have a decreased level in a sample from a mammal that has been exposed to radiation and/or is likely to develop one or more radiation injuries (e.g., CRI and/or ARS) following radiation exposure can be as described in Table 3, Table 5, Table 7, and/or Table 9. For example, a metabolite that can have a decreased level in a sample from a mammal that has been exposed to radiation and/or is likely to develop one or more radiation injuries (e.g., CRI and/or ARS) following radiation exposure can be as shown in FIG. 2D, FIG. 3D, and/or FIG. 5.

Any appropriate sample from a mammal (e.g., a human) can be assessed as described herein (e.g., for the presence, absence, or level of five or more metabolites and/or the presence or absence of one or more enriched metabolic pathways). In some cases, a sample can be a biological sample. In some cases, a sample can contain one or more biological molecules (e.g., nucleic acids such as DNA and RNA, polypeptides, carbohydrates, lipids, hormones, and/or metabolites). Examples of samples that can be assessed as described herein include, without limitation, fluid samples (e.g., whole blood, serum, plasma, urine, cerebrospinal fluid (CSF), and saliva), tissue samples (e.g., jejunum tissues, lung tissues, heart tissues, kidney tissues, skin tissues, bone marrow tissues, gastrointestinal tract tissues, cardiovascular system tissues, and central nervous system tissues), cellular samples (e.g., hematopoietic cells), breathe samples, and fecal samples. A biological sample can be a fresh sample or a fixed sample (e.g., a formaldehyde-fixed sample or a formalin-fixed sample). In some cases, a biological sample can be a processed sample (e.g., to isolate or extract one or more biological molecules). For example, a blood (e.g., plasma) sample can be obtained from a mammal (e.g., a human) and can be assessed for the presence, absence, or level of five or more (e.g., five, six, seven, eight, nine, ten, eleven, or more) metabolites and/or the presence or absence of one or more (e.g., one, two, three, four, five, six, seven, or more) enriched metabolic pathways to determine if the mammal is likely to develop one or more radiation injuries (e.g., CRI and/or ARS) following radiation exposure (e.g., radiation therapy administered as a part of a cancer treatment) based, at least in part, on the presence of an altered level (e.g., an increased level or a decreased level) of five or more metabolites and/or the presence or absence of one or more enriched metabolic pathways in the sample.

Any appropriate method can be used to detect the presence, absence, or level of five or more (e.g., five, six, seven, eight, nine, ten, eleven, or more) metabolites and/or the presence or absence of one or more (e.g., one, two, three, four, five, six, seven, or more) enriched metabolic pathways within a sample (e.g., a sample obtained from a mammal such as a human). For example, mass spectrometry (e.g., liquid chromatography-mass spectrometry (LC-MS)), nuclear magnetic resonance (NMR), gas chromatography-mass spectrometry (GC-MS), capillary electrophoresis-mass spectrometry (CE-MS), and/or Fourier transform infrared spectroscopy (FTIR) can be used to determine the presence, absence, or level of five or more metabolites and/or the presence or absence of one or more enriched metabolic pathways in a sample. In some cases, LC-MS with NMR can be used to determine the presence, absence, or level of five or more metabolites and/or the presence or absence of one or more enriched metabolic pathways in a sample. In some cases, the presence, absence, or level of five or more metabolites and/or the presence or absence of one or more enriched metabolic pathways within a sample can be determined as described in Example 1.

When the presence, absence, or level of two or more (e.g., two, three, four, five, six, seven, or more) metabolites and/or the presence or absence of two or more (e.g., two, three, four, five, six, seven, or more) enriched metabolic pathways within a sample (e.g., a sample obtained from a mammal such as a human) are being detected, the presence, absence, or level of each metabolite and/or each metabolic pathway can be detected in separate assays or in a single assay (e.g., a multiplexed assay).

This document also provides methods and materials for treating a mammal (e.g., a mammal identified as having been exposed to radiation and/or as being likely to develop one or more radiation injuries following radiation exposure). In some cases, a method for treating a mammal (e.g., a mammal identified as having been exposed to radiation and/or as being likely to develop one or more radiation injuries following radiation exposure) can be prophylactic treatment (i.e., delivered prior to any radiation exposure) such as a radio protective treatment. In some cases, a method for treating a mammal (e.g., a mammal identified as having been exposed to radiation and/or as being likely to develop one or more radiation injuries following radiation exposure) can be therapeutic treatment (i.e., delivered after radiation exposure) such as a radio-mitigation agent or a radio-therapeutic agent. For example, a therapeutic treatment can be used to prevent or delay development of one or more radiation injuries (e.g., following radiation exposure). For example, a therapeutic treatment can be used to reduce or eliminate one or more radiation injuries (e.g., following radiation exposure).

In some cases, a mammal (e.g., a human) identified as having been exposed to radiation as described herein (e.g., based, at least in part, on the presence of an altered level of five or more metabolites and/or the presence of one or more enriched metabolic pathways in a sample from the mammal) can be administered one or more (e.g., one, two, three, four, five, six, seven, or more) radiation counter-measures (e.g., radioprotective agents, radio-mitigation agents, and/or radio-therapeutic agents) to treat the mammal. For example, a mammal (e.g., a human) identified as having been exposed to radiation as described herein can be administered or instructed to self-administer one or more radiation counter-measures. A radiation counter-measure that can be used as described herein can be any type of molecule (e.g., small molecules or polypeptides). In some cases, a radiation counter-measure can be a radioprotective agent (e.g., an agent that can be administered to a mammal prior to a radiation exposure to reduce or eliminate the likelihood of the mammal developing symptoms of radiation exposure). For example, a radioprotective agent can be a free radical scavenger (e.g., an antioxidant). For example, a radioprotective agent can reduce or eliminate nucleic acid (e.g., DNA) damage. Examples of radioprotective agents include, without limitation, potassium iodide (KI), amifostine (2-(3-aminpropyl) aminoethylphosphorothioate), Prussian blue, diethylenetriamine pentaacetate (DTPA; e.g., Ca-DTPA and Zn-DTPA), granulocyte colony stimulating factor (G-CSF) such as filgrastim (e.g., Neupogen©). In some cases, a radiation counter-measure can be a radio-mitigation agent (e.g., an agent that can be administered to a mammal following a radiation exposure to reduce or eliminate the likelihood of the mammal developing symptoms of radiation exposure in an asymptomatic mammal or to reduce or eliminate one or more symptoms of radiation exposure in a symptomatic mammal). Examples of radio-mitigation agents include, without limitation, G-CSF such as filgrastim (e.g., NEUPOGEN®), a 20 KDa monomethoxypolyethylene glycol (PEG) molecule covalently linked to the N-terminal methionyl residue of G-CSF (PEGylated G-CSF) such as pegfilgrastim (e.g., NEULASTA®), and granulocyte-macrophage colony-stimulating factor (GM-CSF) such as sargramostim (e.g., LEUKINE®). In some cases, a radiation counter-measure can be a radio-therapeutic agent (e.g., an agent that can be administered to a mammal following a radiation exposure to reduce or eliminate one or more symptoms of radiation exposure and/or provide palliative care). Examples of radio-therapeutic agents include, without limitation, pain relievers (e.g., nonsteroidal anti-inflammatory drugs (NSAIDs) and opioids), hematopoietic stem cells (HSC; e.g., as provided in HSC transplants and blood transfusions), cytokines, and G-CSF such as filgrastim (e.g., NEUPOGEN®), or gene therapy. In some cases, a radiation counter measure can be as described elsewhere (see, e.g., Singh et al., Exp. Opin. Pharmacotherapy, 21:317-337 (2020); and Obrador et al., Biomedicines 8:461; doi:10.3390/biomedicines8110461 (2020)).

In some cases, one or more radiation counter-measures (e.g., radioprotective agents, radio-mitigation agents, and/or radio-therapeutic agents) can be administered to a mammal (e.g., a human) in need thereof (e.g., a mammal identified as having been exposed to radiation as described herein) to delay or prevent the onset of one or more symptoms of radiation exposure. Examples of symptoms of radiation exposure include, without limitation, itching (e.g., itching skin), tingling (e.g., tingling skin), transient erythema, edema, nausea, vomiting, diarrhea, headache, fever, dizziness, disorientation, weakness, fatigue, hair loss, bloody vomit, bloody stool, infections, low blood pressure, loss of appetite, hair loss, damaged sebaceous glands, atrophy, fibrosis, changes (e.g., decreases or increases) in skin pigmentation, ulceration of the exposed tissue, and necrosis of the exposed tissue. For example, one or more radioprotective agents and/or one or more radio-mitigation agents can be administered to a mammal (e.g., a human) in need thereof (e.g., a mammal identified as having been exposed to radiation as described herein) to delay the onset of one or more symptoms of radiation exposure by, for example, 10, 20, 30, 40, 50, 60, 70, 80, 90, 95, or more percent.

In some cases, one or more radiation counter-measures can be administered to a mammal (e.g., a human) in need thereof (e.g., a mammal identified as having been exposed to radiation as described herein) to reduce or eliminate one or more symptoms of radiation exposure. Examples of symptoms of radiation exposure include, without limitation, itching (e.g., itching skin), tingling (e.g., tingling skin), transient erythema, edema, nausea, vomiting, diarrhea, headache, fever, dizziness, disorientation, weakness, fatigue, hair loss, bloody vomit, bloody stool, infections, low blood pressure, loss of appetite, hair loss, damaged sebaceous glands, atrophy, fibrosis, changes (e.g., decreases or increases) in skin pigmentation, ulceration of the exposed tissue, and necrosis of the exposed tissue. For example, one or more radio-therapeutic agents can be administered to a mammal (e.g., a human) in need thereof (e.g., a mammal identified as having been exposed to radiation as described herein) to reduce one or more symptoms of radiation exposure by, for example, 10, 20, 30, 40, 50, 60, 70, 80, 90, 95, or more percent. In some cases, one or more radio-therapeutic agents can be administered to a mammal (e.g., a human) in need thereof (e.g., a mammal identified as having been exposed to radiation as described herein) to increase the reduction of one or more symptoms of radiation exposure by, for example, 10, 20, 30, 40, 50, 60, 70, 80, 90, 95, or more percent (e.g., as compared to a mammal that has been exposed to radiation but has not been administered one or more radiation counter-measures).

In some cases, a mammal (e.g., a human) identified as being likely to develop one or more radiation injuries following radiation exposure as described herein (e.g., based, at least in part, on the presence of an altered level of five or more metabolites and/or the presence of one or more enriched metabolic pathways in a sample from the mammal) can be administered one or more radiation counter-measures (e.g., radioprotective agents, radio-mitigation agents, and/or radio-therapeutic agents) to treat the mammal. For example, a mammal (e.g., a human) identified as being likely to develop one or more radiation injuries following radiation exposure as described herein can be administered or instructed to self-administer one or more (e.g., one, two, three, four, five, or more) radiation counter-measures. For example, one or more radioprotective agents and/or one or more radio-mitigation agents can be administered to a mammal (e.g., a human) in need thereof (e.g., a mammal identified as being likely to develop one or more radiation injuries following radiation exposure as described herein) to reduce the likelihood of developing one or more radiation injuries can occur in the mammal following radiation exposure by, for example, 10, 20, 30, 40, 50, 60, 70, 80, 90, 95, or more percent.

In some cases, the methods and materials provided herein can be used to treat a mammal (e.g., a human) having cancer by administering, to the mammal, one or more cancer treatments that is/are selected based, at least in part, on whether or not the mammal is likely to experience radiation injury (e.g., CRI and/or ARS) when the cancer is treated with radiation therapy. For example, a mammal (e.g., a human) having cancer and identified as being likely to develop one or more radiation injuries following radiation exposure (e.g., radiation therapy administered as a part of a cancer treatment) as described herein (e.g., based, at least in part, on the presence of an altered level of five or more metabolites and/or the presence of one or more enriched metabolic pathways in a sample from the mammal) can be administered one or more alternative cancer treatments (e.g., cancer treatments other than radiation therapies) to treat the mammal. In some cases, a mammal (e.g., a human) having cancer and identified as being likely to develop one or more radiation injuries following radiation exposure as described herein can be administered or instructed to self-administer one or more (e.g., one, two, three, four, five, or more) alternative cancer treatments. Examples of alternative cancer treatments (e.g., cancer treatments other than radiation therapies) include, without limitation, administering one or more anti-cancer agents (e.g., chemotherapeutic agents, targeted cancer drugs, immunotherapy drugs, and hormone therapy drugs), surgery, stem cell transplants, and genetic therapies. For example, one or more alternative cancer treatments can be administered to a mammal (e.g., a human) in need thereof (e.g., a mammal having cancer and identified as being likely to develop one or more radiation injuries following radiation exposure as described herein) to reduce the number of cancer cells in the mammal by, for example, 10, 20, 30, 40, 50, 60, 70, 80, 90, 95, or more percent.

In some cases, a mammal (e.g., a human) having cancer and identified as not being likely to develop one or more radiation injuries following radiation exposure (e.g., radiation therapy administered as a part of a cancer treatment) as described herein (e.g., based, at least in part, on the absence of an altered level of five or more metabolites and/or the absence of one or more enriched metabolic pathways in a sample from the mammal) can be administered one or more radiation therapies to treat the mammal. For example, a mammal (e.g., a human) having cancer and identified as not being likely to develop one or more radiation injuries following radiation exposure as described herein can be administered or instructed to self-administer one or more (e.g., one, two, three, four, five, or more) radiation therapies. Examples of types of radiation therapy include, without limitation, external beam radiation therapy, contact x-ray brachytherapy, brachytherapy (sealed source radiotherapy), unsealed source radiotherapy (systemic radioisotope therapy), intraoperative radiotherapy, and deep inspiration breath-hold. For example, one or more radiation therapies can be administered to a mammal (e.g., a human) in need thereof (e.g., a mammal having cancer and identified as not being likely to develop one or more radiation injuries following radiation exposure as described herein) to reduce the number of cancer cells in the mammal by, for example, 10, 20, 30, 40, 50, 60, 70, 80, 90, 95, or more percent.

In some cases, the methods and materials provided herein can be used to monitor a mammal (e.g., a human such as a human having cancer) for radiation injury following radiation exposure (e.g., radiation therapy administered as a part of a cancer treatment). For example, a mammal having been exposed to radiation can be monitored for the development of one or more radiation injuries as described herein (e.g., based, at least in part, on the absence of an altered level of five or more metabolites and/or the absence of one or more enriched metabolic pathways in a sample from the mammal). When the absence of an altered level (e.g., an increased level or a decreased level) of five or more metabolites and/or the absence of one or more enriched metabolic pathways in a sample from the mammal is detected, the mammal can be identified as not being likely to develop one or more radiation injuries. When the presence of an altered level (e.g., an increased level or a decreased level) of five or more metabolites and/or the presence of one or more enriched metabolic pathways in a sample from the mammal is detected, the mammal can be identified as being likely to develop one or more radiation injuries.

In some cases, the methods and materials provided herein can be used to monitor a mammal (e.g., a human such as a human having cancer) for effectiveness of a treatment with one or more radiation counter measures (e.g., radioprotective agents, radio-mitigation agents, and/or radio-therapeutic agents) following radiation exposure (e.g., radiation therapy administered as a part of a cancer treatment). For example, a mammal having been exposed to radiation and having been administered one or more radiation counter measures can be monitored for the development of one or more radiation injuries as described herein (e.g., based, at least in part, on the presence or absence of an altered level of five or more metabolites and/or the presence or absence of one or more enriched metabolic pathways in a sample from the mammal). When the absence of an altered level (e.g., an increased level or a decreased level) of five or more metabolites and/or the absence of one or more enriched metabolic pathways in a sample from the mammal is detected, the mammal can be identified as not being likely to develop one or more radiation injuries and the radiation counter measures can be determined as likely being an effective treatment for that mammal. When the presence of an altered level (e.g., an increased level or a decreased level) of five or more metabolites and/or the presence of one or more enriched metabolic pathways in a sample from the mammal is detected, the mammal can be identified as being likely to develop one or more radiation injuries and the radiation counter measures can be determined as likely being an ineffective treatment for that mammal. In some cases, when a radiation counter measure is determined as likely being an ineffective treatment for that mammal, the mammal can be administered, or instructed to self-administer one or more different radiation counter measures.

In some cases, the methods and materials provided herein can be used to monitor one or more radiation counter measures (e.g., a radioprotective agents, radio-mitigation agents, and/or radio-therapeutic agents) for efficacy. For example, a candidate radiation counter measure can be administered to cultured cells (e.g., cultured mammalian cells), the cultured cells can be exposed to radiation, and the cells can be assessed for the presence or absence of an altered level (e.g., an increased level or a decreased level) of five or more (e.g., five, six, seven, eight, nine, ten, eleven, or more) metabolites and/or the presence or absence of one or more (e.g., one, two, three, four, five, or more) enriched metabolic pathways. When the reduction or absence of an altered level (e.g., an increased level or a decreased level) of five or more metabolites and/or reduction or absence of one or more enriched metabolic pathways in the cultured cells is detected, the candidate radiation counter measure can be classified as being an effective radiation counter measure. When the presence of an altered level (e.g., an increased level or a decreased level) of five or more metabolites and/or the presence of one or more enriched metabolic pathways in the cultured cells is detected, the candidate radioprotective agent can be classified as not being an effective radiation counter measure. For example, a candidate radiation counter measure can be administered to a non-human mammal, the non-human mammal can be exposed to radiation, and the non-human mammal can be monitored for the presence or absence of an altered level of five or more metabolites and/or the presence or absence of one or more enriched metabolic pathways in a sample from the mammal. When the absence of an altered level (e.g., an increased level or a decreased level) of five or more metabolites and/or the absence of one or more enriched metabolic pathways in a sample from the non-human mammal is detected, the candidate radioprotective agent can be classified as an effective radiation counter measure. When the presence of an altered level (e.g., an increased level or a decreased level) of five or more metabolites and/or the presence of one or more enriched metabolic pathways in a sample from the non-human mammal is detected, the candidate radioprotective agent can be classified as not being an effective radiation counter measure.

In some cases, the methods and materials provided herein can be used to identify one or more radiation counter measures (e.g., radioprotective agents, radio-mitigation agents, and/or radio-therapeutic agents). For example, a candidate radioprotective agent can be administered to a non-human mammal, the non-human mammal can be exposed to radiation, and an altered level (e.g., an increased level or a decreased level) of five or more (e.g., five, six, seven, eight, nine, ten, eleven, or more) metabolites and/or the presence or absence of one or more (e.g., one, two, three, four, five, or more) enriched metabolic pathways in a sample from the non-human mammal can be detected. When the absence of an altered level (e.g., an increased level or a decreased level) of five or more metabolites and/or the absence of one or more enriched metabolic pathways in a sample from the non-human mammal is detected, the candidate radioprotective agent can be classified as a radioprotective agent. When the presence of an altered level (e.g., an increased level or a decreased level) of five or more metabolites in a sample from the non-human mammal is detected, the candidate radioprotective agent is not classified as a radioprotective agent.

The invention will be further described in the following examples, which do not limit the scope of the invention described in the claims.

### EXAMPLES

### Example 1: Radiation Exposure Induces a Similar Time-Dependent Metabolic Change in Both Mice and Non-Human Primates that is Mitigated by Amifostine

This Example identifies metabolite biomarkers that can be used to diagnosis exposure to radiation, characterizes metabolic changes that result from radiation exposure prior to the development of ARS, and evaluates the potential of amifostine as a radioprotector for ARS.

### Materials and Methods

### ARS Animal Model Cohorts

The metabolomics study utilized three distinct animal cohorts (Table 1). Cohort one comprised serum from mice exposed to ⁶⁰Co-γ-radiation. Briefly, male CD2FI mice (Mus musculus) aged 12-14 weeks were housed under conventional conditions in microisolator filter-top cages in a facility fully-accredited by AAALCA. Animals were acclimated 1 to 2 weeks before sham treatment or exposure to radiation. Total-body irradiation (TBI) was carried out in a single dose of 14 Gy at a rate of 0.6 Gy/minute in well-ventilated Plexiglas boxes to a midline tissue in the Armed Forces Radiobiology Research Institute's (AFRRI)⁶⁰Co-γ-irradiation. A total of 236 serum samples were obtained and divided into two groups. Sham (Sh) mice were not exposed to radiation and test mice (Gy) were exposed to a single dose of 14 Gy radiation. Serum samples were collected from 16 mice at five time-points: 5 Hours (5H), day 1 (D1), day 2 (D2), day 3 (D3) and day 4 (D4) post radiation exposure. Each group contained 8 biological samples with 3 analytical replicates each for a total of 24 replicates per group with the following exceptions: Sh1 n=22, GyD3 n=23 and GyD4 n=19.

TABLE 1
Demographic and sample information for three ARS animal model cohorts.

| | | | Time Points | | | | |
|---|---|---|---|---|---|---|---|
| Animal Model | Fluid Type | Radiation Exposure | Day 0 | Day 1 | Day 2 | Day 3 | Day 4 |
| Mouse | Serum | Control (CD) | 24 | 22 | 24 | 24 | 24 |
| n = 236 | | 14Gy | 24 | 24 | 24 | 23 | 19 |
| | | | Day −7 | 8 Hours | Day 2 | Day 3 | Day 8 |
| Non-Human | Serum | 5.8Gy | 7 | 7 | 7 | 7 | 7 |
| Primates | | 7.2Gy | 7 | 7 | 7 | 7 | 7 |
| n = 70 | | | | | | | |
| | | | Day −5 | Day 0 | Day 1 | Day 6 | Day 9 |
| Mouse | Blood | Am50 | 12 | 12 | 12 | 12 | 12 |
| n = 300 | | Am200 | 12 | 12 | 12 | 12 | 12 |
| | | RAD (9.6Gy) | 12 | 12 | 12 | 12 | 12 |
| | | RAD + Am50 (9.6Gy) | 12 | 12 | 12 | 12 | 12 |
| | | RAD + Am200 (9.6Gy) | 12 | 12 | 12 | 12 | 12 |

Cohort two comprised serum from NHPs exposed to two discrete levels of ⁶⁰Co-γ-radiation. Briefly, male and female naïve rhesus macaques (Macaca mulatta, Chinese substrain) aged 3-5 years and weighing 3.6 to 8.4 kg were housed in facilities accredited by the AAALAC International. NHPs were exposed to 5.8 Gy (LD_{30/60}) or 7.2 Gy (LD_{70/60}) at a rate of 0.6 Gy/minute dose of ionizing radiation. A total of 70 NHP serum samples were obtained from 14 NHPs. The NHP serum samples were collected at five time points: a control sample collected prior to radiation exposure at day −7 (D−7), and four time-points post-radiation exposure at 8 hours (8H), day 2 (D2), day 3 (D3) and day 8 (D8).

Cohort three comprised whole blood samples from mice exposed to a single dose of 9.6 Gy ⁶⁰Co-γ-radiation at a rate of 0.6 Gy/minute in the presence or absence of drug intervention (50 mg/kg or 200 mg/kg amifostine). A total of 300 blood samples were collected from 60 mice divided into five groups (12 mice per group). The five groups correspond to: (1) amifostine dosage of 50 mg/kg without radiation exposure, (2) amifostine dosage of 200 mg/kg without radiation exposure, (3) amifostine dosage of 50 mg/kg with radiation exposure, (4) amifostine dosage of 200 mg/kg with radiation exposure, and (5) a radiation exposure without an amifostine treatment. Blood samples were collected at five time-points: at day −5 (D−5), at time of amifostine dosage, day −1 (D−1), day 1 (D1), day 5 (D5) and day 9 (D9). Amifostine was delivered through a subcutaneous injection.

### Chemicals

All chemicals were obtained from Sigma Aldrich (Milwaukee, WI) unless otherwise denoted. 3-(trimethylsilyl) propionic-2,2,3,3-D₄ acid sodium salt (98% D, TMSP) was purchased from Cambridge Isotopes (Andover, MA). Potassium phosphate dibasic salt (anhydrous, 99.1% pure) and monobasic salt (crystal, 99.8% pure) were purchased from Fisher Scientific (Fair Lawn, NJ).

### Preparation of Blood and Serum Samples for Metabolomics Analysis

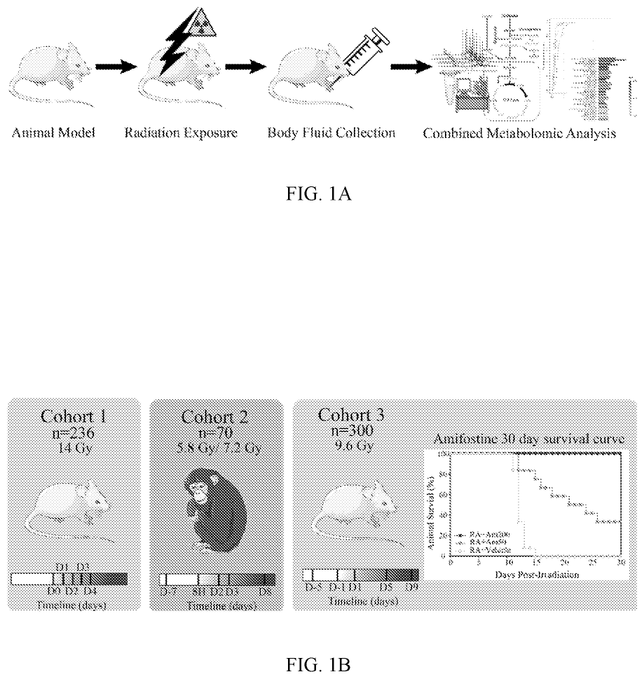

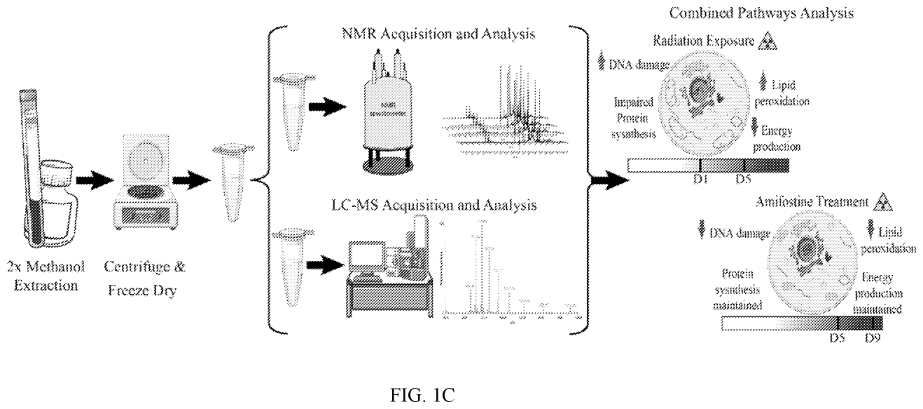

Mouse (n=236) and NHP (n=70) serum samples, and mouse blood samples (n=300) were simultaneously prepared for LC-MS and NMR analysis as shown in FIG. 1C. 2.0% by volume of NaN₃ was added to each serum or blood sample to avoid bacterial growth. The protein component was precipitated by adding 100 μL (2×) of methanol to 50 μL of serum or blood. Deoxygenated methanol was used for the blood preparation to avoid unnecessary oxidation of the red blood cells. Methanol was purged of oxygen by bubbling in nitrogen gas for 30 minutes.

The 1:2 mixtures were then vortexed for 10 seconds. Serum samples were incubated at room temperature for 5 minutes. Blood samples were sonicated in a water bath for 10 minutes at 4° C., followed by incubation at −20° C. for 20 minutes. The samples were then centrifuged at 15,000×g for 20 minutes at 4° C. to pellet the proteins. The supernatant was collected, transferred to an Eppendorf tube, and centrifuged again at 15,000×g for 5 minutes at 4° C. The supernatant was then divided 20% and 80% for LC-MS and NMR samples, respectively. Samples were snap-frozen in liquid nitrogen. Methanol was evaporated by speed vacuum centrifugation (SpeedVac R Plus, Savant) and water was removed by lyophilization using FreeZone™ (Labconco, Kansas City, MO).

### NMR Data Collection and Processing

Dried serum or blood samples were reconstituted using 50 μL of 50 mM phosphate buffer in 100% D20 at pH 7.2 (uncorrected) with 50 μM TMSP as an NMR chemical shift reference and internal standard. The samples were centrifuged at 15,000×g for 20 minutes at 4° C. to remove any particulates, and the supernatant was transferred to 1.7 mm NMR tube for data acquisition. All NMR experiments were collected at 298K with a Bruker AVANCE III HD 700 MHz spectrometer equipped with a 5 mm quadruple resonance QCI-P cryoprobe (¹H, ¹³C, ¹⁵N and ³¹P) with z-axis gradients. A SampleJet automated sample changer with Bruker ICON-NMR software was used to collect all data.

A one-dimensional (1D)¹H NMR spectrum was collected for each sample. 1D ¹H NMR spectra were collected with 32K data points, a spectrum width of 8417.5 Hz, 256 scans, and 4 dummy scans using an excitation sculpting pulse sequence to remove the solvent peak (see, for example, Nguyen, B. D.; Meng, X.; Donovan, K. J.; Shaka, A. J., Journal of Magnetic Resonance 2007, 184 (2), 263-274). In addition, a natural abundance 2D ¹H-¹³C HSQC spectrum was collected for representatives from each group. The 2D ¹H-¹³C HSQC spectra were collected with 64 scans, 16 dummy scans, and a 2 second relaxation delay. The spectra were collected with 2K data points and a spectrum width of 11160 Hz in the direct dimension, and 1024 data points and a spectrum width of 29052 Hz in the indirect dimension. The 2D ¹H-¹³C HSQC spectra were collected with 25% sparsity using the deterministic non-uniform sampling (NUS) schedule (see, for example, Worley, B.; Powers, R., Journal of Magnetic Resonance 2015, 261, 19-26).

The 2D ¹H-¹³C HSQC NUS spectra were reconstructed with the MDD algorithm as described elsewhere (see, for example, Orekhov, V. Y.; Jaravine, V. A., Progress in Nuclear Magnetic Resonance Spectroscopy 2011, 59, 271-292). Both 1D and 2D NMR spectra were processed using NMRPipe or MVAPACK software toolkit. Each 1D ¹H NMR spectrum was Fourier transformed, phased, referenced to TMSP and aligned with icoshift to obtain a data matrix as described elsewhere (Savorani, F.; Tomasi, G.; Engelsen, S. B., Journal of Magnetic Resonance 2010, 202 (2), 190-202). The 1D ¹H NMR data matrix was then binned using the intelligent adaptive binning algorithm, UV scaling, and normalized using the Probabilistic Quotient (PQ) method. The 2D ¹H-¹³C NMR data was Fourier transformed and phased. The 2D ¹H-¹³C HSQC spectra were visualized and peak-picked using NMRviewJ (version 8.0). Discriminatory features were identified to specific metabolites using Chenomx Suite 8.0, Human Metabolome Database (HMDB), SpinAssin, and BMRB. A ¹H and ¹³C chemical shift error of 0.08 and 0.25 ppm, respectively, was used to match experimental chemical shifts to reference spectra.

### LC-MS Data Collection and Processing

For LC-MS analysis, dried metabolome extracts were reconstituted in 20 μL of 0.1% formic acid and centrifuged at 15,000×g at 4° C. for 10 minutes to remove particulates. Quality control (QC) samples were prepared by pooling 5 μL from each experimental metabolomics sample. An LC-MS spectrum was acquired for five QC samples prior to analyzing the metabolome extracts. In addition, two QC samples were analyzed after every 12 injections of experimental metabolomics samples to monitor system stability.

The metabolome analyses were performed using an ACQUITY Ultra-Performance Liquid Chromatography (UPLC) system (Waters, Milford, MA, USA) coupled to a Waters Xevo G2-XS Q-TOF mass spectrometer (Waters Co., Milford, MA, USA.) with an electrospray ionization (ESI) mode. An ACQUITY UPLC HSS T3 C18 (1.0×50 mm, 1.8 pm, Waters Co., Milford, MA, USA) column was used. Column and autosampler temperatures were set to 40° C. and 8° C., respectively. The flow rate was set to 100 μL/minutes. The mobile phase was composed of 0.10% formic acid in water (A) and 0.1% formic acid in acetonitrile (B). 2 μL of a metabolome extract or a QC sample were injected and separated with a linear gradient program from 1% to 95% B in 7.30 minutes, held at 95% for 1.5 minutes and re-equilibrate for 1.2 minutes.

The ESI source was set to positive mode, and the capillary and cone voltages were set to 3.15 kV and 40 V, respectively. The source temperature was set to 120° C., the cone gas flow was set to 50 L/hour, and the desolvation temperature and flow rate were set to 300° C. and 857 L/hour, respectively. Data collection was in the data-independent acquisition mode (MSE). The quadrupole was set to transfer all ions and alternate collision energy from low (4 eV) to high (ramped from 15 to 45 eV) energies with a m z scan range of 50 to 2000 Da. The time of flight mass analyzer was calibrated with an external mass calibrant (Leucine-Enkephalin [M+H]⁺=556.2771) infused via the Lock-mass channel.

Following acquisition, raw LC-MS data were imported into Progenesis QI v.2.1 (Non-linear Dynamics, Newcastle, UK) for automatic alignment, detection and deconvolution. 2D ion intensity maps were generated and the retention times were aligned using a QC pooled spectrum as a reference. The peak detection parameters were as follows: the sensitivity method value was set to 3, no minimum peak width and no retention time limits were used. Adducts of the same compound were automatically grouped during deconvolution using a list ([M+H]⁺, [M+Na]⁺, [M+K], [M+NH₄]⁺, [M+H—H₂O]⁺, [M+H-2H₂O]⁺, [M+ACN+H]⁺, [M+CH₃OH+H]⁺ and [M+ACN+Na]⁺) of adducts pre-defined from a raw data import. Normalization was computed to the mean log ratio (MLR) algorithm in Progenesis. Features not present in at least 50% of one experimental group or not present in the QC spectrum with a coefficient of variation (CV) of <30% were removed. Batch or run order variance were removed with LOESS QC corrections. Putative annotation of metabolites was performed by MetaScope with a theoretical fragment search against HMDB. The putative metabolite assignment was performed according to parameters, including Score, Fragmentation score, and Isotope similarity by Progenesis QI. Only statistically significant features were submitted for identification.

### NMR and MS Data Analyses

All unsupervised and supervised multivariate statistics were conducted using MVAPACK in OCTAVE (4.4.1). Unless otherwise stated, unit variance (UV) scaling was applied to both X and Y inputs before NIPAL implementation for Partial Least Squares/Projection to Latent Structures (PLS) and Orthogonal Projection to Latent Structures (OPLS) with successive iterations halted based on the cross-validated (7-fold CV) and fraction of Y variation modelled (Q²). CV-ANOVA, permutation testing (n=1000), and Variable Importance in Projection scores (VIP >1) were used for model validation and evaluation, respectively. Shared and Unique (SUS) principles using p(corr) were adopted to facilitate model interpretation. All univariate statistics were conducted using ‘Limma’ or ‘Stats’ in R (3.5.2). Unless otherwise stated, log 2 transformations were applied before concurrent linear fits by generalized least squares and computed moderated T-statistics, F-statistics and log-odds by empirical Bayes moderation of standard errors to a common value (Limma). Respective p-values were adjusted for multiple testing by Benjamini-Hochberg (BH) and q-values calculated to the expected false discovery rate (FDR), with significance to <0.05.

MetaboAnalyst software was used to access ‘Pathways Analysis’ and ‘Pathways Enrichment’ of all significantly altered metabolites, as described elsewhere (see, for example, Chong, J.; Wishart, D. S.; Xia, J., Current Protocols in Bioinformatics 2019, 68 (1)).

### Results

Biofluids from ARS Animal Models Enabled a Search for Metabolite Biomarkers of Radiation Exposure

As outlined in FIG. 1A, the experimental design consisted of biofluid collected from two ARS animal models pre- and post-radiation exposure. The species information, specific radiation exposure, and biofluid collection time points are summarized in FIG. 1B and Table 1. Specifically, the metabolomics study consisted of three distinct cohorts (Cohorts 1, 2 and 3) comprising either a mouse or NHP ARS model. The animals were exposed to one of four different ⁶⁰Co-γ-radiation doses (5.8, 7.2, 9.6 and 14 Gy) at a rate of 0.6 Gy/minutes; and two amifostine treatments doses and a control (0, 50 or 200 mg/kg), a potential radioprotective agent. Serum or blood samples were collected over a range of five different time-points per cohort. Serum or blood collected prior to radiation exposure were used as Shams or unirradiated controls. Serum or blood collected post-irradiation were used to monitor the impact on the mice and NHPs metabolome relative to controls. The metabolomes were then extracted from the biofluids for a combined analysis using NMR and LC-MS as shown in FIG. 1C.

Cohort one comprised mice serum samples exposed to 14 Gy ⁶⁰Co-γ-radiation, which provides the basis set to obtain a metabolomics signature of radiation exposure in mice. Cohort one contained a total of 236 serum samples divided into two groups. The sham (Sh, n=8) or control mice were not exposed to radiation and the treated mice (Gy, n=8) were exposed to a single 14 Gy dose of γ-radiation. In general, each group contained a total of 24 replicates consisting of 8 biological samples with 3 analytical replicates per biological sample. Serum samples were collected at five different time points corresponding to 5 hours, and at 1, 2, 3, and 4 days post-irradiation (Table 1).

Extending the study to a second species, NHP were utilized to ascertain if a metabolic response to radiation exposure was species specific or if there were species-independent metabolome changes. Cohort two consisted of NHP groups (n=7) exposed to a midline dose of either 5.8 Gy (LD_{30/60}) or 7.2 Gy (LD_{70/60}). A correlation between radiation dosage and metabolome changes was investigated; and whether the metabolic changes were proportional to dosage or required a minimal dose to be impacted was assessed. Cohort 2 consisted of a total of 70 serum samples collected at five different time points. Specifically, a serum sample was collected from each NHP seven days prior to irradiation to serve as a sham control, and subsequent serum samples were collected 8 hours, and 2, 3, and 8 days post-irradiation for metabolomics analysis (Table 1).

Another distinct aspect of the metabolomics study was the inclusion of a third cohort, which was an extended replication of cohort one. The repeatability and the reliability of the mouse metabolic changes resulting from irradiation, and, by inference, the NHP dysregulated metabolism consistent with the mouse model was tested. The radiation dosage was reduced to 9.6 Gy (LD_{90/30}) to extend the study (survivability) out to Day 9. This would allow for a comparison to the Day 8 time point from the NHP cohort. It also extended the investigation into a correlation between radiation dosage and metabolome changes.

Lastly, to evaluate the radioprotective impact of amifostine treatment, cohort three also contained mice that received a subcutaneous injection of either 50 mg/kg or 200 mg/kg of amifostine 30 minutes (+10 minutes) before exposure to radiation. A total of 300 samples were collected from 60 mice divided into five groups (n=12): (1) 50 mg/kg amifostine treatment without radiation exposure (Am50), (2) 200 mg/kg amifostine treatment without radiation exposure (Am200), (3) only 9.6 Gy radiation exposure (RAD), (4) 9.6 Gy radiation exposure with a 50 mg/kg amifostine treatment (RAD+Am50), and (5) 9.6 Gy radiation exposure with a 200 mg/kg amifostine treatment (RAD+Am200). Serum samples were collected from each group at five different time points. Serum collected at five days and one day prior to radiation exposure were used as controls, and serum samples for metabolomics analysis were collected at 1, 5 and 9 days post-irradiation (Table 1).

Thirty-day survival data on amifostine treatment of cohort 3 (FIG. 1B) demonstrates that at 9.6 Gy ⁶¹Co-γ-radiation without preventive treatment 100% of animals died as a result of radiation exposure. 50 mg/kg of amifostine treatment resulted in 40% survival at 30 days post-irradiation. Furthermore, an incremental decrease in survival began at day 11. Treatment with 200 mg/kg amifostine 30 minutes prior to radiation exposure showed the most promising results with 100% animal survival 30 days post-irradiation. These data demonstrate that amifostine treatment can be an effective radioprotective agent in a dose dependent manner.

Results of these metabolomics studies on radiation exposure and amifostine treatment ultimately demonstrated a dose dependent effect of amifostine as well as the temporal effects of radiation and amifostine treatment as a radioprotective drug. As shown in FIG. 1C, radiation exposure of all three cross-species cohorts displayed dysregulated pathways between D1 and D5 after radiation exposure. Consistent pathways associated with radiation exposure included lipid degradation, impaired protein synthesis, and decreased TCA activity with downstream effects of amino acid metabolism and energy production. On the other hand, amifostine treatment resulted in preliminary recovery of the dysregulated pathways between D5 and D9 after radiation exposure. Animal models treated with 200 mg/kg amifostine showed recovery of pathways including lipid metabolism, glycolysis, and TCA activity as well as energy and amino acid metabolism.

### Statistical Model to Evaluate Temporal Metabolite Changes Due to Radiation Exposure

To examine whether the height of perturbation (radiation exposure—effect of interest) equates to maximum observed metabolic difference from the norm, a test condition was assigned to each cohort. For the mouse serum dataset (Cohort 1), the test condition was assigned to the 14 Gy radiation exposure. For the NHP serum dataset (Cohort 2), the test condition was assigned to the 5.8 Gy and 7.2 Gy radiation exposures, and, for Cohort 3, the mouse blood samples with the amifostine treatment and 9.6 Gy radiation exposure served as the test condition. Both the NMR and LC-MS data across all their respective time-points were modelled against a control free from perturbation, which served as the baseline. One predictive component (PC) was then used from the partial least squares (PLS) model to construct metabolite trajectories. The resultant Q² values from the PLS models were used as a proxy for metabolic perturbations of interest, and once mapped across the time points allowed comparable trajectories to be interrogated. The downstream analyses were then directed to focus only on important comparisons. This subset of conditions best explained the goals of the study and primarily used traditional univariate statistical methods to make observations and conclusions.

### Mouse Serum Highlights Temporal Metabolic Trajectories Linked to Radiation Exposure

Cohort 1—Mouse Serum (14 Gy). For both NMR and LC-MS, all radiation (Gy, test) and Sham (Sh, control) time-points (day 1(D1), day 2 (D2), day 3 (D3), day 4 (D4)) were modeled separately against each baseline norm of 5 hours (5H) with PLS (1 PC). For clarification, Gy D1 to D4 were compared to Gy5H, and Sh D1 to D4 were compared to Sh5H. The resulting Q² statistics were then visualized across time to map the trajectories (FIG. 2A: NMR and FIG. 3: LC-MS). The metabolic trajectory was nicely reproduced for Gy when the baseline norm of Gy5H was replaced with Sh5H.

Analyses of NMR data from mice serum samples showed that 14 Gy exposed and Sham mice experienced different non-stationary metabolic time trajectories. This is evident by comparing the top (14 Gy) and bottom (Sham) panels in FIG. 2A. Exposure to 14 Gy radiation invoked a dual response in the mouse metabolome, which occurred at an early time point (#1, D1) of 1 day following radiation and at a later time point (#2, D4) 4 days post-irradiation. Response #1 at D1 (p=2.83×10⁻⁸) and response #2 at D4 (p=1.33×10⁻¹⁰) were both significant. Conversely, Sham intervention exhibited only a single response at D2 (Q²>0.40). A similar response was observed in analysis of LC-MS data (FIG. 3A).

The application of Shared and Unique Structures (SUS) principles allowed characterization of the significant responses when the 5H baseline was compared within its Gy or Sham group, as well as comparisons between GyD4, GyD1 or ShD2 (FIG. 3B). In a SUS-plot, shared structures are along the diagonal and unique structures are off-diagonal. Each within comparison displayed abundant shared structures, and good correlation (pcorr), particularly when a threshold was set to variable importance score greater than 1 (VIP >1). Comparisons between GyD1 and GyD4 displayed abundant unique structures; whereas, a comparison between GyD1 and ShD2 displayed abundant shared structures shown along the diagonal. The latter comparisons inferred the Sham D2 response was independent of radiation and was likely due to stress, albeit delayed, as variables decrease or increase together across models. LC-MS data highlighted similar effects of interest at D1 (p=1.22×10⁻¹⁰), D4 (p=1.11×10⁻¹⁶) and D2 for radiation (Gy) and Sham mice. A similar SUS pattern could also be observed, with a stronger correlation between GyD1 and ShD2 than between GyD1 and GyD4, when filtered for VIP >1 (FIG. 3B). Thus, subsequent analyses were constrained to GyD4 vs 5H as the main radiation response.

Significant PLS models (Q²>0.40, p<0.05) were recalculated (Monte Carlo simulations) to evaluate and summarize parameters (Q², pcorr, VIP) with confidence (95% CI) shown in FIG. 3C. A total of 34 NMR probabilistic quotient (PQ) bins and 1630 MS mean log ratio peaks (MLR) were found to be significant features to group separation and radiation perturbation (VIP >1). Univariate statistics were adopted to further filter significant NMR and MS spectral features and calculate traditional T-test statistics (p<0.05) and fold changes (FC >1) for GyD4 as shown in FIG. 1B and FIG. 3C, respectively. A total of 29 NMR and 1,584 MS spectral features retained differential significance from the baseline norm (5H) to GyD4. A majority of the features (66%) were downregulated for NMR contrary to a majority (59%) upregulated for MS at D4. Metabolite identification of these features resulted in a total of 95 putative metabolite identifications (12 from NMR and 83 from MS) as shown in Table 2. Heatmaps of NMR and MS features show distinct clustering of metabolites at the 5H and D4 time points (FIG. 2C and FIG. 4, respectively). A MetaboAnalyst pathway enrichment analysis of these 95 metabolites resulted in 49 potentially perturbed metabolic pathways as shown in FIG. 2D and Table 3.

TABLE 3
Perturbed Pathways of Cohort 1.

| Enriched Pathways | FC^{a} | p-value | FDR^{b} |
|---|---|---|---|
| Methylhistidine Metabolism | 0.250 | 0.299 | 1 |
| Glucose-Alanine Cycle | 0.231 | 0.0909 | 1 |
| Transfer of Acetyl Groups into Mitochondria | 0.136 | 0.285 | 1 |
| Ammonia Recycling | 0.125 | 0.286 | 1 |
| Beta Oxidation of Very Long Chain Fatty | 0.118 | 0.431 | 1 |
| Acids | | | |
| Alanine Metabolism | 0.118 | 0.431 | 1 |
| Phenylacetate Metabolism | 0.111 | 0.552 | 1 |
| Lactose Degradation | 0.111 | 0.552 | 1 |
| Phenylalanine and Tyrosine Metabolism | 0.107 | 0.43 | 1 |
| Urea Cycle | 0.103 | 0.454 | 1 |
| Pyruvaldehyde Degradation | 0.100 | 0.59 | 1 |
| Carnitine Synthesis | 0.091 | 0.571 | 1 |
| D-Arginine and D-Ornithine Metabolism | 0.091 | 0.625 | 1 |
| Taurine and Hypotaurine Metabolism | 0.083 | 0.658 | 1 |
| Glycolysis | 0.080 | 0.643 | 1 |
| Oxidation of Branched Chain Fatty Acids | 0.077 | 0.664 | 1 |
| Thyroid hormone synthesis | 0.077 | 0.687 | 1 |
| Histidine Metabolism | 0.070 | 0.725 | 1 |
| Warburg Effect | 0.069 | 0.745 | 1 |
| Tryptophan Metabolism | 0.067 | 0.769 | 1 |
| Citric Acid Cycle | 0.063 | 0.773 | 1 |
| Glutamate Metabolism | 0.061 | 0.805 | 1 |
| Amino Sugar Metabolism | 0.061 | 0.788 | 1 |
| Gluconeogenesis | 0.057 | 0.815 | 1 |
| Nicotinate and Nicotinamide Metabolism | 0.054 | 0.839 | 1 |
| Glycine and Serine Metabolism | 0.051 | 0.894 | 1 |
| Pyrimidine Metabolism | 0.051 | 0.894 | 1 |
| Catecholamine Biosynthesis | 0.050 | 0.834 | 1 |
| Riboflavin Metabolism | 0.050 | 0.834 | 1 |
| Lactose Synthesis | 0.050 | 0.834 | 1 |
| Sphingolipid Metabolism | 0.050 | 0.87 | 1 |
| Valine, Leucine and Isoleucine Degradation | 0.050 | 0.901 | 1 |
| Glutathione Metabolism | 0.048 | 0.848 | 1 |
| Tyrosine Metabolism | 0.042 | 0.956 | 1 |
| Cysteine Metabolism | 0.038 | 0.904 | 1 |
| Mitochondrial Beta-Oxidation of Short Chain | 0.037 | 0.912 | 1 |
| Saturated Fatty Acids | | | |
| Selenoamino Acid Metabolism | 0.036 | 0.92 | 1 |
| Mitochondrial Beta-Oxidation of Long | 0.036 | 0.92 | 1 |
| Chain Saturated Fatty Acids | | | |
| Pterine Biosynthesis | 0.034 | 0.927 | 1 |
| Beta-Alanine Metabolism | 0.029 | 0.954 | 1 |
| Aspartate Metabolism | 0.029 | 0.958 | 1 |
| Fatty Acid Biosynthesis | 0.029 | 0.958 | 1 |
| Retinol Metabolism | 0.027 | 0.965 | 1 |
| Purine Metabolism | 0.027 | 0.991 | 1 |
| Galactose Metabolism | 0.026 | 0.968 | 1 |
| Propanoate Metabolism | 0.024 | 0.978 | 1 |
| Fatty acid Metabolism | 0.023 | 0.98 | 1 |
| Pyruvate Metabolism | 0.021 | 0.987 | 1 |
| Arginine and Proline Metabolism | 0.019 | 0.992 | 1 |

ªFC—fold change, potentially enriched pathways ordered by fold change impact.
^{b}FDR—false discovery rate

### Metabolic Trajectories in NHP are Radiation Dose Dependent

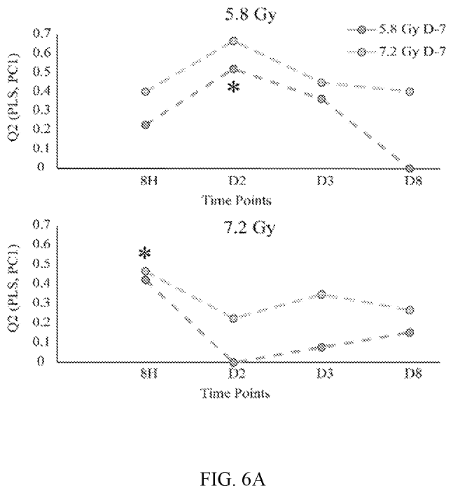

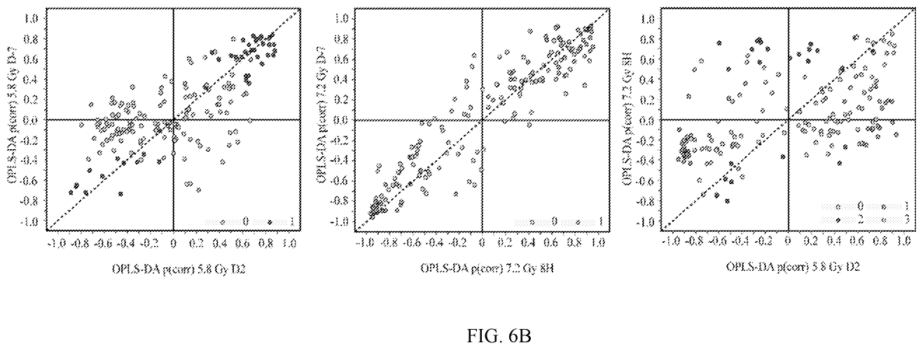

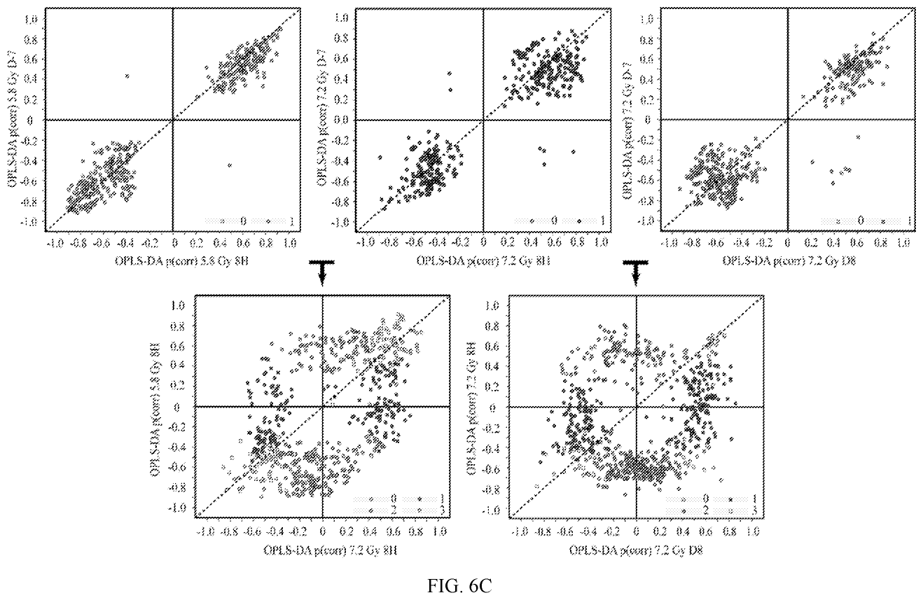

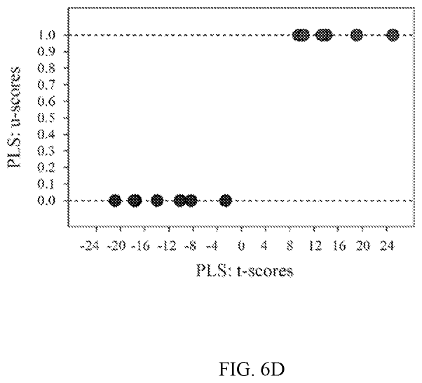

Cohort 2—NHP Serum (5.8 Gy and 7.2 Gy). For both LC-MS and NMR, all 5.8 Gy and 7.2 Gy time points (8 hours (8H), day 2 (D2), day 3 (D3), day 8 (D8)) were modeled with PLS (1 PC) against the baseline norm of 7 days prior to irradiation (D−7). The resulting Q² statistics from the LC-MS and NMR data sets were then visualized across time to map trajectories (FIG. 5A and FIG. 6A, respectively). The metabolic trajectories were reproduced for the 5.8 Gy and 7.2 Gy conditions when the D−7 baseline norms were interchanged.

As shown in FIG. 6A, the NMR trajectories showed that NHP experienced a single response to both radiation exposures (5.8 Gy and 7.2 Gy). However, the extent of the perturbation for 5.8 Gy was delayed to D2 (p=2.25×10⁻³), compared to 8H (p=1.62×10⁻²) for 7.2 Gy. This delayed response suggests a dose-dependent metabolic response to radiation exposure. A supplementary pairwise time-point comparison was made between the 5.8 Gy and 7.2 Gy data sets. The OPLS-DA (1PC with varying OC) models were only valid for the D−7 and 8H time points. At all other time-points, 5.8 Gy and 7.2 Gy treated NHP could not be discriminated. SUS principles were then applied to analyze and further characterize the significant responses for the D−7 within comparisons (5.8 Gy and 7.2 Gy), and the comparisons between 5.8 Gy D2 and 7.2 Gy 8H (FIG. 6B). Each D−7 within comparison displayed an abundant shared structure. Conversely, the comparison between 5.8 Gy D2 and 7.2 Gy 8H displayed only minimal shared structures, which is indicative of a response independent of radiation exposure and was likely due to stress (FIG. 6B). A response to radiation would be expected to possess ample consistency between the two doses (5.8 Gy and 7.2 Gy). With the shared structures absent between comparisons, NMR alone appears insufficient to characterize the metabolic changes in NHP serum due to radiation exposure at 5.8 Gy and 7.2 Gy.

LC-MS data showed that 5.8 Gy and 7.2 Gy exposed NHP experienced different, non-stationary metabolic time trajectories (FIG. 5A). Exposure to 7.2 Gy radiation invoked a dual response at 8H (p=4.70×10⁻³) and D8 (p=5.30×10⁻³). 5.8 Gy only exhibited a single response at 8H (Q²>0.30). An observation consistent with a metabolic dose dependent response.

A subsequent pairwise time-point comparison between the 5.8 Gy and 7.2 Gy data sets was conducted using OPLS-DA (1PC with varying OC). The resulting OPLS-DA models proved valid for D−7, 8H and D8. At all other time-points, 5.8 Gy and 7.2 Gy exposed NHP could not be discriminated. When filtered for VIP >1, SUS correlations were stronger between 5.8 Gy 8H and 7.2 Gy 8H than between 7.2 Gy 8H and 7.2 Gy D8 (FIG. 6C). Thus, subsequent metabolomic analysis was constrained to a comparison between D−7 and 7.2 Gy D8 as the main response to radiation exposure. Significant PLS models (Q²>0.30, p<0.05) were recalculated with Monte Carlo simulations to evaluate and summarize parameters (Q², pcorr, VIP) with confidence (95% CI) shown in FIG. 6D. A total of 406 MS MLR peaks were found to be significant contributors to group separation and radiation perturbation (VIP >1). Univariate statistics were adopted to further filter significant MS discriminators and calculate traditional T-test statistics and fold changes for 7.2 Gy D8 (FIG. 5B). A total of 92 MS spectral features retained differential significance from the baseline norm (D−7) to 7.2 Gy D8 (p<0.05). A majority of the MS spectral features were downregulated (80%) at D8 compared to D−7. Metabolite identification of these MS features resulted in a total of 16 putative metabolite identifications as shown in Table 4. Heatmap of MS features show distinct clustering of metabolites at the 7.2 Gy D−7 and D8 time points (FIG. 5C). MetaboAnalyst pathway enrichment analysis of these 16 metabolites identified 29 potentially perturbed metabolic pathways as shown in FIG. 5D and Table 5.

TABLE 5
Perturbed Pathways of Cohort 2.

| Enriched Pathways | FCª | p-value | FDR^{b} |
|---|---|---|---|
| Glycerol Phosphate Shuttle | 0.091 | 0.160 | 1.000 |
| Beta Oxidation of Very Long Chain Fatty | 0.059 | 0.237 | 1.000 |
| Acids | | | |
| Vitamin B6 Metabolism | 0.050 | 0.272 | 1.000 |
| Lactose Synthesis | 0.050 | 0.272 | 1.000 |
| Glutathione Metabolism | 0.048 | 0.284 | 1.000 |
| Carnitine Synthesis | 0.045 | 0.295 | 1.000 |
| Purine Metabolism | 0.041 | 0.103 | 1.000 |
| Glycolysis | 0.040 | 0.329 | 1.000 |
| Cysteine Metabolism | 0.038 | 0.339 | 1.000 |
| Oxidation of Branched Chain Fatty Acids | 0.038 | 0.339 | 1.000 |
| Inositol Phosphate Metabolism | 0.038 | 0.339 | 1.000 |
| Mitochondrial Beta-Oxidation of Short Chain | 0.037 | 0.350 | 1.000 |
| Saturated Fatty Acids | | | |
| Selenoamino Acid Metabolism | 0.036 | 0.360 | 1.000 |
| Mitochondrial Beta-Oxidation of Long Chain | 0.036 | 0.360 | 1.000 |
| Saturated Fatty Acids | | | |
| Urea Cycle | 0.034 | 0.371 | 1.000 |
| Pyrimidine Metabolism | 0.034 | 0.234 | 1.000 |
| Ammonia Recycling | 0.031 | 0.401 | 1.000 |
| Fructose and Mannose Degradation | 0.031 | 0.401 | 1.000 |
| Inositol Metabolism | 0.030 | 0.410 | 1.000 |
| Gluconeogenesis | 0.029 | 0.429 | 1.000 |
| Nicotinate and Nicotinamide Metabolism | 0.027 | 0.447 | 1.000 |
| Propanoate Metabolism | 0.024 | 0.491 | 1.000 |
| Fatty acid Metabolism | 0.023 | 0.499 | 1.000 |
| Pyruvate Metabolism | 0.021 | 0.539 | 1.000 |
| Glutamate Metabolism | 0.020 | 0.546 | 1.000 |
| Arginine and Proline Metabolism | 0.019 | 0.575 | 1.000 |
| Warburg Effect | 0.017 | 0.609 | 1.000 |
| Valine, Leucine and Isoleucine Degradation | 0.017 | 0.622 | 1.000 |
| Arachidonic Acid Metabolism | 0.014 | 0.675 | 1.000 |

ªFC—fold change, potentially enriched pathways ordered by fold change impact.
^{b}FDR—false discovery rate

### Amifostine Imparts Radioprotection in a Dose Dependent Manner

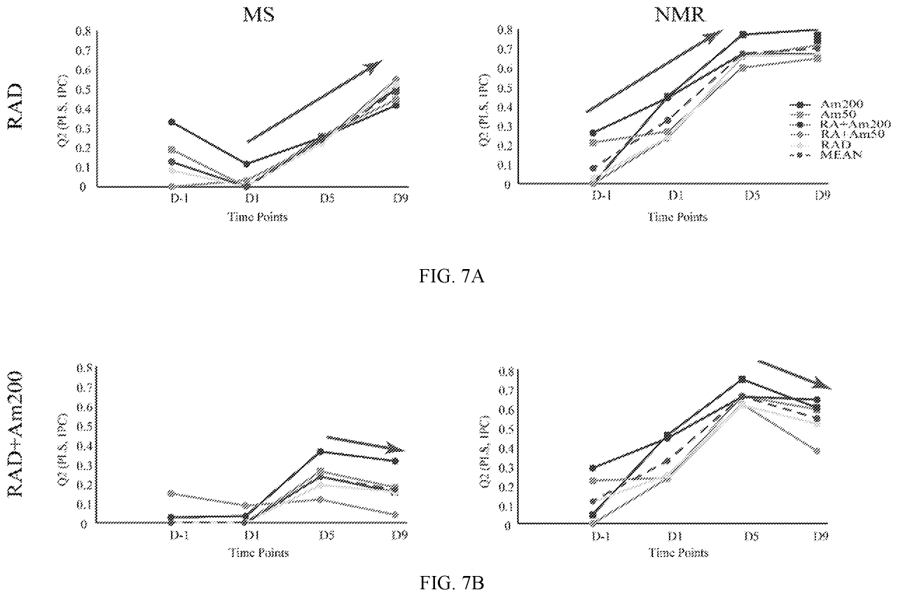

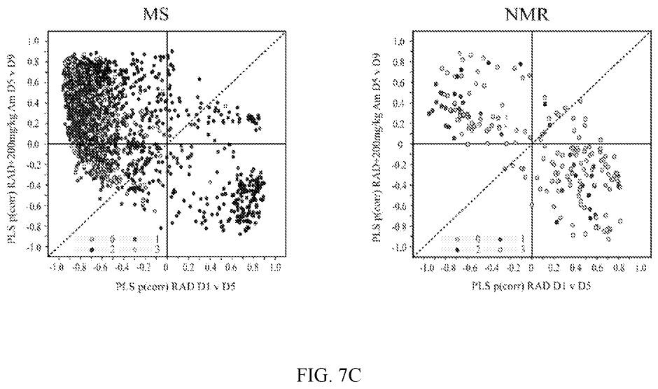

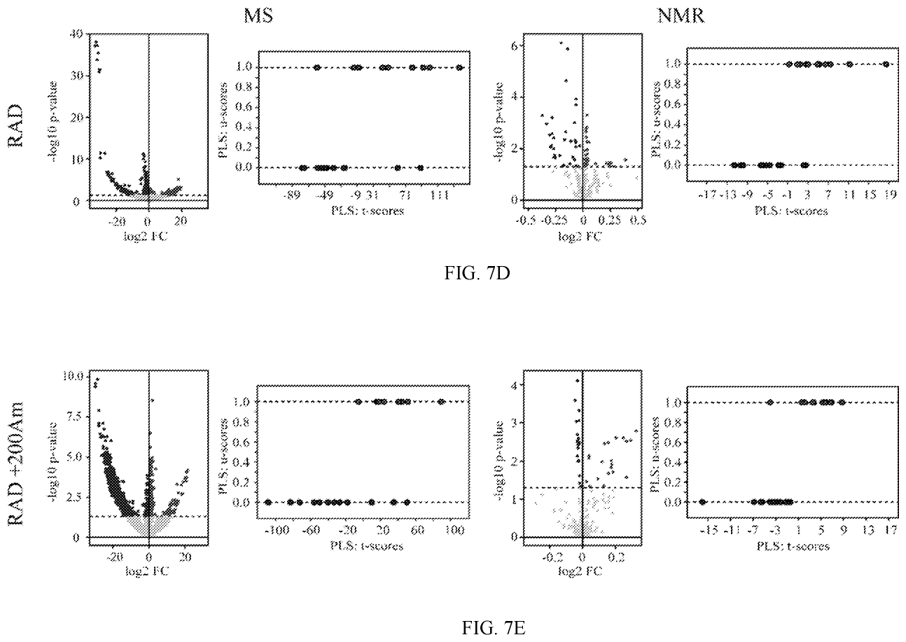

Cohort 3—Mouse Blood (9.6 Gy). The third cohort consisted of mouse whole blood samples after a dose of 9.6 Gy irradiation. Prior to irradiation some of the mice received an amifostine treatment at either a low (50 mg/kg) or high (200 mg/kg) dose. The strategy for data analysis remained consistent with the previous two cohorts. For both NMR and LC-MS, all time-points (day −1 (D−1), day 1 (D1), day 5 (D5), day 9 (D9)) were modeled with PLS (1 PC) against the baseline norm of day −5 (D−5). The resulting Q² statistics were then visualized across time to map trajectories (FIG. 7A-B). Once again, the metabolic trajectories could be nicely reproduced for both conditions when the baseline norms were interchanged. The NMR results showed that an exposure to 9.6 Gy radiation (0 mg/kg amifostine) invoked a single, incremental response throughout time that peaked at D9 (p=1.74×10⁻¹⁴). LC-MS data confirmed an equivalent metabolic time trajectory towards radiation response at D9 (p=6.39×10⁻⁴) as shown in FIG. 7A.

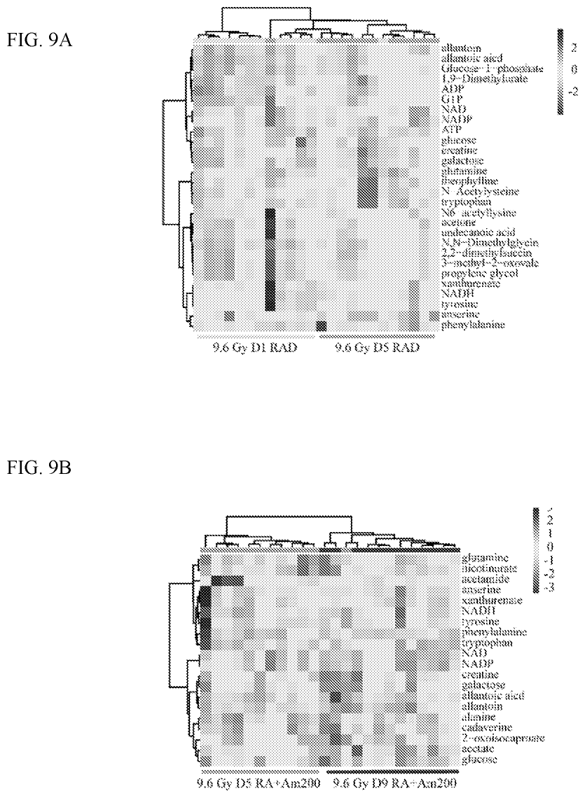

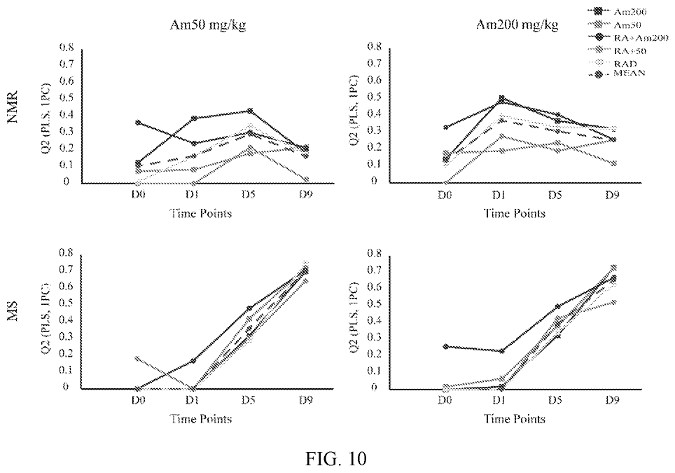

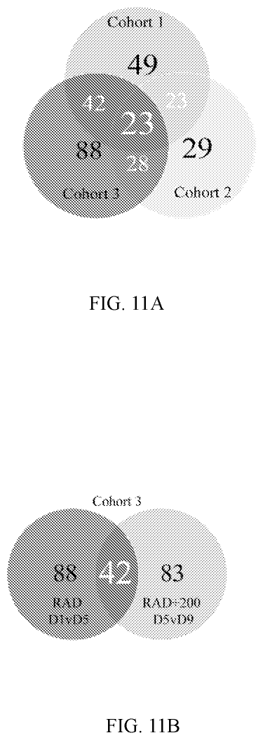

The sharpest inclines (metabolome changes) occurred at post-irradiation timepoints between D1 vs D5 and D5 vs D9. SUS analysis of the NMR and LC-MS data sets displayed abundant shared structure, though anti-correlated (pcorr), between the significant responses, particularly with a threshold set at VIP >1 (FIG. 7C). Thus, subsequent analysis was constrained to a comparison of 9.6 Gy D1 to D5 as the main response to radiation. Significant PLS models (Q²>0.30, p<0.05) were recalculated (Monte Carlo simulations) to evaluate and summarize parameters (Q², pcorr, VIP) with confidence (95% CI) as shown in UvT PLS scores plots FIG. 7D. A total of 77 NMR PQN bins and 2,969 MS MLR peaks were found to be significant contributors to group separation and radiation perturbation (VIP >1). Univariate statistics were adopted to further filter significant NMR and MS discriminators and to calculate traditional T-test statistics and fold changes for 9.6 Gy D5 (FIG. 7D). A total of 65 NMR and 916 MS spectral features retained differential significance from D1 to D5 (p<0.05). An essentially even distribution of down- and up-regulated NMR features (51/49) were observed compared to a down-regulated majority (92%) of MS features at D5. Metabolite identification of these features resulted in a total of 34 putative metabolite identifications as shown in Table 6. A heatmap of radiation exposure showed metabolite clustering among both LC-MS (FIG. 8A) and NMR (FIG. 9F). MetaboAnalyst pathway enrichment of these 34 metabolites resulted in 88 potentially perturbed pathways as shown in FIG. 8B and Table 7.

TABLE 7
Perturbed Pathways of Cohort 3 RAD_D1vD5

| Enriched Pathways | FC^{a} | p-value | FDR^{b} |
|---|---|---|---|
| Lactose Degradation | 0.444 | 0.000 | 0.006 |
| Glucose-Alanine Cycle | 0.308 | 0.001 | 0.010 |
| Transfer of Acetyl Groups into Mitochondria | 0.273 | 0.000 | 0.004 |
| Nucleotide Sugars Metabolism | 0.250 | 0.000 | 0.010 |
| Thiamine Metabolism | 0.222 | 0.033 | 0.080 |
| De Novo Triacylglycerol Biosynthesis | 0.222 | 0.033 | 0.080 |
| Mitochondrial Electron Transport Chain | 0.211 | 0.003 | 0.016 |
| Ethanol Degradation | 0.211 | 0.003 | 0.016 |
| Glycerolipid Metabolism | 0.200 | 0.001 | 0.010 |
| Glycolysis | 0.200 | 0.001 | 0.010 |
| Threonine and 2-Oxobutanoate Degradation | 0.200 | 0.003 | 0.017 |
| Malate-Aspartate Shuttle | 0.200 | 0.041 | 0.094 |
| Phytanic Acid Peroxisomal Oxidation | 0.192 | 0.001 | 0.010 |
| Betaine Metabolism | 0.190 | 0.004 | 0.020 |
| Fructose and Mannose Degradation | 0.188 | 0.000 | 0.010 |
| Glycerol Phosphate Shuttle | 0.182 | 0.049 | 0.094 |
| Trehalose Degradation | 0.182 | 0.049 | 0.094 |
| Cardiolipin Biosynthesis | 0.182 | 0.049 | 0.094 |
| Beta Oxidation of Very Long Chain Fatty Acids | 0.176 | 0.017 | 0.050 |
| Folate Metabolism | 0.172 | 0.002 | 0.014 |
| Gluconeogenesis | 0.171 | 0.001 | 0.010 |
| Phosphatidylethanolamine Biosynthesis | 0.167 | 0.057 | 0.100 |
| Nicotinate and Nicotinamide Metabolism | 0.162 | 0.001 | 0.010 |
| Galactose Metabolism | 0.158 | 0.001 | 0.010 |
| Butyrate Metabolism | 0.158 | 0.023 | 0.063 |
| Citric Acid Cycle | 0.156 | 0.003 | 0.017 |
| Cysteine Metabolism | 0.154 | 0.009 | 0.035 |
| Ketone Body Metabolism | 0.154 | 0.066 | 0.108 |
| Lactose Synthesis | 0.150 | 0.026 | 0.069 |
| Mitochondrial Beta-Oxidation of Short | 0.148 | 0.010 | 0.039 |
| Chain Saturated Fatty Acids | | | |
| Pyruvate Metabolism | 0.146 | 0.001 | 0.010 |
| Mitochondrial Beta-Oxidation of Long | 0.143 | 0.012 | 0.040 |
| Chain Saturated Fatty Acids | | | |
| Glutathione Metabolism | 0.143 | 0.030 | 0.076 |
| Phosphatidylcholine Biosynthesis | 0.143 | 0.076 | 0.118 |
| Histidine Metabolism | 0.140 | 0.002 | 0.014 |
| Pterine Biosynthesis | 0.138 | 0.013 | 0.042 |
| Urea Cycle | 0.138 | 0.013 | 0.042 |
| Carnitine Synthesis | 0.136 | 0.034 | 0.080 |
| Starch and Sucrose Metabolism | 0.129 | 0.017 | 0.050 |
| Sphingolipid Metabolism | 0.125 | 0.009 | 0.035 |
| Ammonia Recycling | 0.125 | 0.019 | 0.054 |
| Androstenedione Metabolism | 0.125 | 0.042 | 0.094 |
| Estrone Metabolism | 0.125 | 0.042 | 0.094 |
| Biotin Metabolism | 0.125 | 0.237 | 0.302 |
| Glutamate Metabolism | 0.122 | 0.004 | 0.020 |
| Warburg Effect | 0.121 | 0.002 | 0.014 |
| Beta-Alanine Metabolism | 0.118 | 0.023 | 0.063 |
| Alanine Metabolism | 0.118 | 0.107 | 0.151 |
| Phosphatidylinositol Phosphate Metabolism | 0.118 | 0.107 | 0.151 |
| Oxidation of Branched Chain Fatty Acids | 0.115 | 0.052 | 0.094 |
| Inositol Phosphate Metabolism | 0.115 | 0.052 | 0.094 |
| Plasmalogen Synthesis | 0.115 | 0.052 | 0.094 |
| Arginine and Proline Metabolism | 0.113 | 0.006 | 0.028 |
| Mitochondrial Beta-Oxidation of Medium | 0.111 | 0.057 | 0.100 |
| Chain Saturated Fatty Acids | | | |
| Phenylacetate Metabolism | 0.111 | 0.263 | 0.326 |
| Phenylalanine and Tyrosine Metabolism | 0.107 | 0.062 | 0.103 |
| Selenoamino Acid Metabolism | 0.107 | 0.062 | 0.103 |
| Pentose Phosphate Pathway | 0.103 | 0.068 | 0.109 |
| Glycine and Serine Metabolism | 0.102 | 0.011 | 0.039 |
| Tryptophan Metabolism | 0.100 | 0.012 | 0.040 |
| Lysine Degradation | 0.100 | 0.074 | 0.116 |
| Riboflavin Metabolism | 0.100 | 0.140 | 0.193 |
| Propanoate Metabolism | 0.095 | 0.046 | 0.094 |
| Pantothenate and CoA Biosynthesis | 0.095 | 0.152 | 0.207 |
| Purine Metabolism | 0.095 | 0.009 | 0.035 |
| Methionine Metabolism | 0.093 | 0.050 | 0.094 |
| Fatty acid Metabolism | 0.093 | 0.050 | 0.094 |
| Inositol Metabolism | 0.091 | 0.093 | 0.137 |
| Amino Sugar Metabolism | 0.091 | 0.093 | 0.137 |
| Androgen and Estrogen Metabolism | 0.091 | 0.093 | 0.137 |
| Sulfate/Sulfite Metabolism | 0.091 | 0.164 | 0.220 |
| Degradation of Superoxides | 0.091 | 0.312 | 0.377 |
| Fatty Acid Elongation In Mitochondria | 0.086 | 0.106 | 0.151 |
| Valine, Leucine and Isoleucine Degradation | 0.083 | 0.044 | 0.094 |
| Caffeine Metabolism | 0.083 | 0.188 | 0.245 |
| Retinol Metabolism | 0.081 | 0.121 | 0.169 |
| Bile Acid Biosynthesis | 0.077 | 0.059 | 0.101 |
| Vitamin K Metabolism | 0.071 | 0.379 | 0.447 |
| Steroidogenesis | 0.070 | 0.168 | 0.222 |
| Phospholipid Biosynthesis | 0.069 | 0.250 | 0.314 |
| Steroid Biosynthesis | 0.063 | 0.210 | 0.271 |
| Aspartate Metabolism | 0.057 | 0.325 | 0.389 |
| Spermidine and Spermine Biosynthesis | 0.056 | 0.458 | 0.522 |
| Pyrimidine Metabolism | 0.051 | 0.311 | 0.377 |
| Porphyrin Metabolism | 0.050 | 0.387 | 0.452 |
| Ubiquinone Biosynthesis | 0.050 | 0.494 | 0.557 |
| Tyrosine Metabolism | 0.042 | 0.433 | 0.499 |
| Arachidonic Acid Metabolism | 0.014 | 0.910 | 1.000 |

ªFC—fold change, potentially enriched pathways ordered by fold change impact.
^{b}FDR—false discovery rate

Following radiation exposure (9.6 Gy), profiling of whole blood by NMR showed that untreated (0 mg/kg) and amifostine treated (50 mg/kg and 200 mg/kg) mice experienced different, non-stationary metabolic time trajectories (FIGS. 7A-B). While trajectories at the low amifostine dose (50 mg/kg) could not be discriminated from untreated controls, the high amifostine dose (200 mg/kg) reversed progression back towards the baseline norm (D−5) by D9 (p=1.60×10⁻⁷) as shown in FIG. 73B. These results are consistent with a metabolic dose-dependent response to amifostine treatment.

The LC-MS data showed an identical pattern in response to amifostine treatment with a divergence of trajectories from D5 to D9 (p=2.10×10⁻²). Accordingly, the subsequent analysis was constrained to comparing 9.6 Gy D9 to D5 as the main amifostine response. Significant PLS models (Q²>0.30, p<0.05) were recalculated (Monte Carlo simulations) to evaluate and summarize parameters (Q², pcorr, VIP) with confidence (95% CI) as shown in UvT PLS scores plots FIG. 7E. A total of 64 NMR PQN bins and 3,568 MS MLR peaks were found to be significant contributors to group separation and radiation perturbation (VIP >1). Univariate statistics were adopted to further filter significant NMR and MS discriminators and to calculate T-test statistics and fold changes for D9 (200 mg/kg amifostine and 9.6 Gy) as shown in FIG. 7E. A total of 43 NMR and 1,986 MS spectral features retained differential significance from D5 to D9 (p<0.05). An essentially even distribution of down- and up-regulated (53/47) NMR features were identified, but an up-regulated majority (64%) of MS features were observed at D9. Metabolite identification of these features resulted in a total of 52 putative metabolite identifications as shown in Table 8. A heatmap of radiation exposure in addition to treatment with 200 mg/kg amifostine showed metabolite clustering among both LC-MS (FIG. 8C) and NMR (FIG. 9B). MetaboAnalyst pathway enrichment analysis of these 52 metabolites resulted in 83 potentially perturbed metabolic pathways as shown in FIG. 8D and Table 9.

TABLE 9
Perturbed Pathways of Cohort 3 RAD + 200_D5vD9

| Enriched Pathways | FC^{a} | p-value | FDR^{b} |
|---|---|---|---|
| Glucose-Alanine Cycle | 0.462 | 0.000 | 0.000 |
| Malate-Aspartate Shuttle | 0.300 | 0.006 | 0.094 |
| Lactose Degradation | 0.222 | 0.047 | 0.220 |
| De Novo Triacylglycerol Biosynthesis | 0.222 | 0.047 | 0.220 |
| Glutathione Metabolism | 0.190 | 0.008 | 0.094 |
| Carnitine Synthesis | 0.182 | 0.010 | 0.094 |
| Transfer of Acetyl Groups into Mitochondria | 0.182 | 0.010 | 0.094 |
| Glycerol Phosphate Shuttle | 0.182 | 0.068 | 0.224 |
| Cardiolipin Biosynthesis | 0.182 | 0.068 | 0.224 |
| Beta-Alanine Metabolism | 0.176 | 0.002 | 0.054 |
| Urea Cycle | 0.172 | 0.005 | 0.094 |
| Lysine Degradation | 0.167 | 0.005 | 0.094 |
| Glutamate Metabolism | 0.163 | 0.000 | 0.022 |
| Ethanol Degradation | 0.158 | 0.037 | 0.218 |
| Ketone Body Metabolism | 0.154 | 0.092 | 0.233 |
| Nucleotide Sugars Metabolism | 0.150 | 0.042 | 0.218 |
| Threonine and 2-Oxobutanoate Degradation | 0.150 | 0.042 | 0.218 |
| Folate Metabolism | 0.138 | 0.025 | 0.178 |
| Nicotinate and Nicotinamide Metabolism | 0.135 | 0.014 | 0.121 |
| Ammonia Recycling | 0.125 | 0.035 | 0.218 |
| Androstenedione Metabolism | 0.125 | 0.067 | 0.224 |
| Estrone Metabolism | 0.125 | 0.067 | 0.224 |
| Biotin Metabolism | 0.125 | 0.280 | 0.473 |
| Amino Sugar Metabolism | 0.121 | 0.039 | 0.218 |
| Glycerolipid Metabolism | 0.120 | 0.074 | 0.224 |
| Glycolysis | 0.120 | 0.074 | 0.224 |
| Alanine Metabolism | 0.118 | 0.146 | 0.317 |
| Tryptophan Metabolism | 0.117 | 0.008 | 0.094 |
| Histidine Metabolism | 0.116 | 0.025 | 0.178 |
| Cysteine Metabolism | 0.115 | 0.082 | 0.224 |
| Phytanic Acid Peroxisomal Oxidation | 0.115 | 0.082 | 0.224 |
| Plasmalogen Synthesis | 0.115 | 0.082 | 0.224 |
| Mitochondrial Beta-Oxidation of Short Chain | 0.111 | 0.090 | 0.233 |
| Saturated Fatty Acids | | | |
| Phenylacetate Metabolism | 0.111 | 0.309 | 0.488 |
| Homocysteine Degradation | 0.111 | 0.309 | 0.488 |
| Phenylalanine and Tyrosine Metabolism | 0.107 | 0.098 | 0.233 |
| Selenoamino Acid Metabolism | 0.107 | 0.098 | 0.233 |
| Mitochondrial Beta-Oxidation of Long | 0.107 | 0.098 | 0.233 |
| Chain Saturated Fatty Acids | | | |
| Galactose Metabolism | 0.105 | 0.061 | 0.224 |
| Butyrate Metabolism | 0.105 | 0.174 | 0.342 |
| Mitochondrial Electron Transport Chain | 0.105 | 0.174 | 0.342 |
| Warburg Effect | 0.103 | 0.024 | 0.178 |
| Pterine Biosynthesis | 0.103 | 0.106 | 0.247 |
| Propanoate Metabolism | 0.095 | 0.082 | 0.224 |
| Betaine Metabolism | 0.095 | 0.204 | 0.384 |
| Arginine and Proline Metabolism | 0.094 | 0.056 | 0.224 |
| Fructose and Mannose Degradation | 0.094 | 0.132 | 0.302 |
| Androgen and Estrogen Metabolism | 0.091 | 0.142 | 0.316 |
| Degradation of Superoxides | 0.091 | 0.363 | 0.548 |
| Fatty Acid Elongation In Mitochondria | 0.086 | 0.161 | 0.329 |
| Gluconeogenesis | 0.086 | 0.161 | 0.329 |
| Glycine and Serine Metabolism | 0.085 | 0.081 | 0.224 |
| Pyrimidine Metabolism | 0.085 | 0.081 | 0.224 |
| Caffeine Metabolism | 0.083 | 0.249 | 0.428 |
| Purine Metabolism | 0.081 | 0.069 | 0.224 |
| Retinol Metabolism | 0.081 | 0.181 | 0.348 |
| Thyroid hormone synthesis | 0.077 | 0.414 | 0.597 |
| Sphingolipid Metabolism | 0.075 | 0.213 | 0.393 |
| Mitochondrial Beta-Oxidation of Medium | 0.074 | 0.295 | 0.488 |
| Chain Saturated Fatty Acids | | | |
| Vitamin K Metabolism | 0.071 | 0.438 | 0.613 |
| Methionine Metabolism | 0.070 | 0.245 | 0.428 |
| Fatty acid Metabolism | 0.070 | 0.245 | 0.428 |
| Steroidogenesis | 0.070 | 0.245 | 0.428 |
| Tyrosine Metabolism | 0.069 | 0.155 | 0.329 |
| Phospholipid Biosynthesis | 0.069 | 0.325 | 0.506 |
| Starch and Sucrose Metabolism | 0.065 | 0.355 | 0.544 |
| Pyruvate Metabolism | 0.063 | 0.301 | 0.488 |
| Citric Acid Cycle | 0.063 | 0.370 | 0.549 |
| Beta Oxidation of Very Long Chain Fatty | 0.059 | 0.504 | 0.676 |
| Acids | | | |
| Aspartate Metabolism | 0.057 | 0.414 | 0.597 |
| Valine, Leucine and Isoleucine Degradation | 0.050 | 0.436 | 0.613 |
| Porphyrin Metabolism | 0.050 | 0.483 | 0.667 |
| Catecholamine Biosynthesis | 0.050 | 0.562 | 0.715 |
| Ubiquinone Biosynthesis | 0.050 | 0.562 | 0.715 |
| Lactose Synthesis | 0.050 | 0.562 | 0.715 |
| Bile Acid Biosynthesis | 0.046 | 0.490 | 0.667 |
| Sulfate/Sulfite Metabolism | 0.045 | 0.597 | 0.750 |
| Arachidonic Acid Metabolism | 0.043 | 0.532 | 0.704 |
| Oxidation of Branched Chain Fatty Acids | 0.038 | 0.659 | 0.807 |
| Inositol Phosphate Metabolism | 0.038 | 0.659 | 0.807 |
| Pentose Phosphate Pathway | 0.034 | 0.699 | 0.846 |
| Inositol Metabolism | 0.030 | 0.746 | 0.892 |
| Steroid Biosynthesis | 0.021 | 0.866 | 1.000 |

ªFC—fold change, potentially enriched pathways ordered by fold change impact.
^{b}FDR—false discovery rate

For completeness, NMR results in the absence of 9.6 Gy radiation showed that mice treated with both amifostine doses experienced a stationary metabolic time trajectory. The trajectory differs in intercept (mean), which suggested no interaction with time (FIG. 10). Conversely, the LC-MS data showed that both amifostine treatments produced a non-stationary metabolic time trajectory that peaked at D9 (p=1.52×10⁻⁶ and p=1.83×10⁻⁵, respectively) as shown in FIG. 10. As expected, the significant responses were easily discriminated from exposure to radiation independent of amifostine dose. The two doses of amifostine could not be differentiated over the 9 days in either the NMR or LC-MS data sets in the absence of 9.6 Gy radiation.

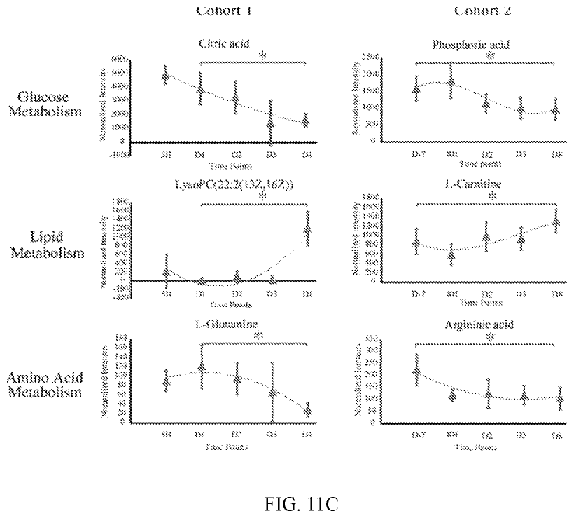

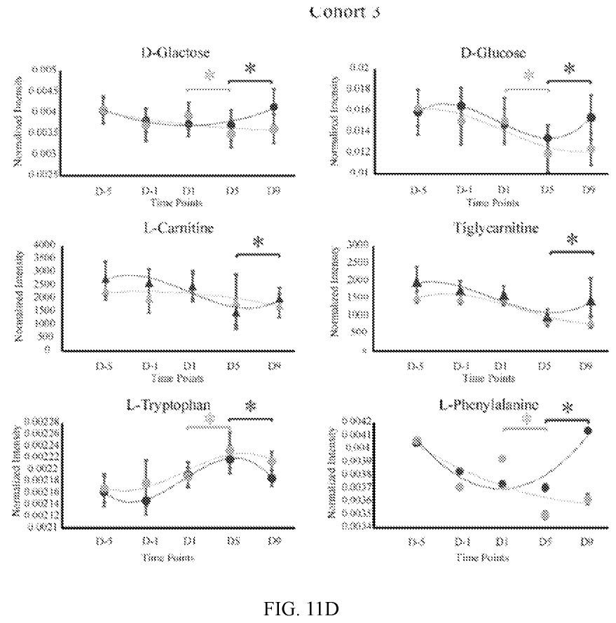

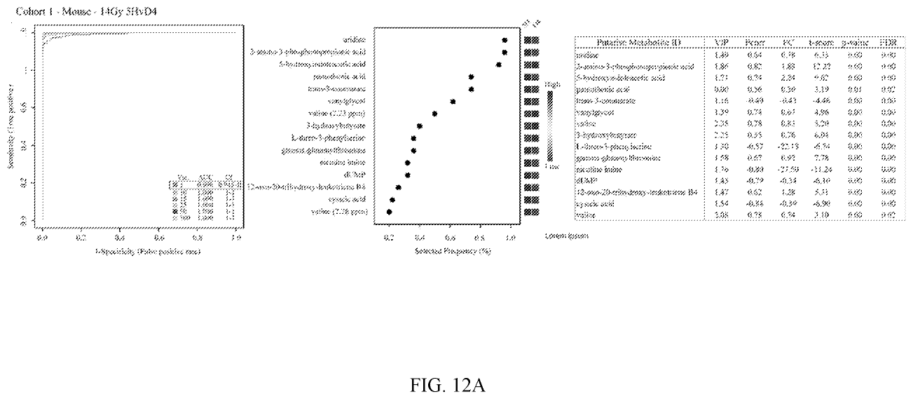

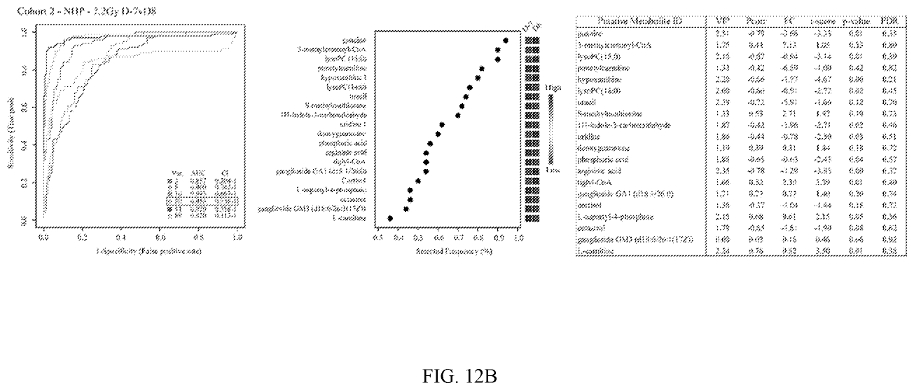

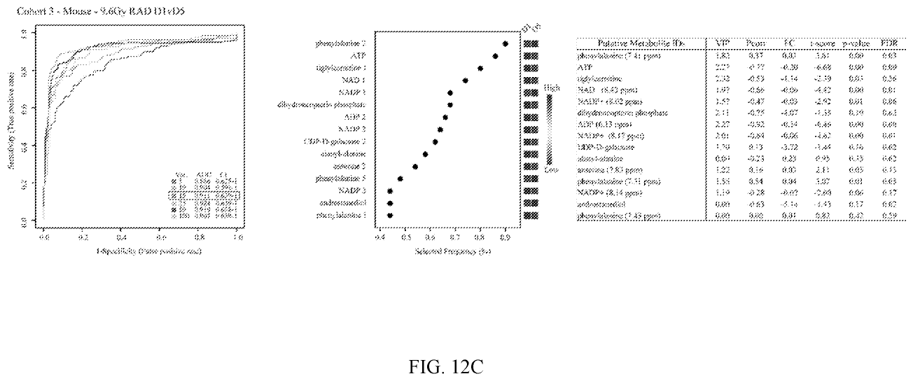

Across all three cohorts, a total of 23 metabolic pathways were found to be uniformly perturbed as a result of radiation exposure (FIG. 11A). Major pathways based on fold change and p-value calculations were shown to be glucose metabolism, de-novo lipid synthesis and metabolism, and amino acid metabolism (Tables 3, 5, and 7). As illustrated by representative metabolites (FIG. 11C), radiation exposure induced a decrease in glucose metabolism, amino acid metabolism and an increase in lipid synthesis.

### Example 2: Exemplary Embodiments

Embodiment 1. A method for determining whether or not a mammal is likely to develop a radiation injury following radiation exposure, wherein said method comprises:
(a) detecting a presence or absence of an altered level of at least five metabolites in a sample from said mammal;
(b) classifying said mammal as being likely to develop a radiation injury following radiation exposure if said presence of said altered level is detected; and
(c) classifying said mammal as not being likely to develop a radiation injury following radiation exposure if said altered level is not detected.

Embodiment 2. The method of embodiment 1, wherein said altered level of said at least five metabolites is an increased level, and wherein said at least five metabolites are selected from the group consisting of 10,11-dihydro-12r-hydroxy-leukotriene E4, 12-oxo-20-trihydroxy-leukotriene B4, 2-amino-3-phosphonopropionic acid, 3-hydroxybutyrate, 5-hydroxyindoleacetic acid, 5-hydroxytryptophol glucuronide, 7-methylguanosine, acetyldigitoxin, alpha-CEHC, D-glucose, formamidopyrimidine nucleoside triphosphate, gamma glutamylglutamic acid, gamma-glutamylthreonine, ganglioside GA1 (d18:1/16:0), ganglioside GM2 (d18:1/18:1(11z)), L-beta-aspartyl-L-threonine, leucyl-valine, leukotriene F4, L-isoleucine, L-leucine, lysoPC(22:2(13z,16z)), N-a-acetyl-L-arginine, N-acetyltryptophan, N-gamma-L-glutamyl-D-alanine, PG(18:3(9Z,12Z,15Z)/20:3(5Z,8Z,11Z)), phenylalanylphenylalanine, phosphoribosyl-AMP, p-hydroxyphenylacetic acid, PS(14:0/14:1(9Z)), PS(20:4(5Z,8Z,11Z,14Z)/14:1(9Z)), pyruvate, tetrahexosylceramide (d18:1/16:0), trans-3-hydroxycotinine glucuronide, trimethylamine N-oxide, uridine, valine, vanylglycol, L-carnitine, tiglyl-CoA, 2,2-dimethylsuccinic acid, 3-methyl-2-oxovalerate, anserine, L-phenylalanine, L-tryptophan, N,N-Dimethylglycine, N6-acetyllysine, N-acetylysteine, propylene glycol, undecanoic acid, and xanthurenate.

Embodiment 3. The method of embodiment 1, wherein said altered level of said at least five metabolites is a decreased level, and wherein said at least five metabolites are selected from the group consisting of 1-methylhistidine, 2-aminomuconic acid semialdehyde, 2-methylguanosine, 2-oxoarginine, 2-pyrroloylglycine, 3-(3,5-diiodo-4-hydroxyphenyl)pyruvate, 3-dehydroxycarnitine, 3-hydroxyoctanoyl carnitine, 3-nitrotyrosine, 3′-O-methyladenosine, 4-guanidinobutanoic acid, 7-methylguanine, alanyl-serine, allantoic acid, argininic acid, arginyl-methionine, citric acid, creatine, cysteic acid, cysteinyl-cysteine, DL-2-aminooctanoic acid, dUMP, gamma-glutamyltyrosine, glutaminyl-lysine, glutarylcarnitine, glycerophosphocholine, glyceryl lactopalmitate, hippuric acid, IDP, L-acetylcarnitine, L-alanine, L-aspartyl-4-phosphate, L-carnitine, L-glutamine, L-histidine, L-isoleucine, L-phenylalanine, L-tyrosine, melatonin, N1-methyl-2-pyridone-5-carboxamide, N6,N6,N6-trimethyl-L-lysine, N-acetyl-L-methionine, nicotine imine, N-ribosylhistidine, O-isobutyryl-L-carnitine, phenol sulphate, phenylpyruvic acid, picolinoylglycine, proline betaine, pyrogallol-2-O-glucuronide, riboflavin, serinyl-hydroxyproline, sphinganine 1-phosphate, trans-3-coumarate, tyrosyl-glutamate, urocanic acid, 1H-indole-3-carboxaldehyde, 2-hydroxyadenine, allodesmosine, arginyl-histidine, guanine, hypoxanthine, LysoPC(14:0), LysoPC(15:0), LysoPC(18:3(6Z,9Z,12Z)), LysoPE(0:0/18:1(11Z)), phosphoric acid, pyrrolidine, uridine, 1,9-dimethylurate, 2-(3-carboxy-3-aminopropyl)-L-histidine, adenosine diphosphate ribose, adenosine thiamine triphosphate, ADP, ATP, cholesterol glucuronide, D-galactose, D-glucose, GTP, malonyl-carnitin, N-acetyl-D-glucosamine, NAD⁺, NADH, NADP⁺, N-decanoylglycine, phosphoribosyl-AMP, purine, and tiglylcarnitine.

Embodiment 4. The method of any one of embodiments 1-3, wherein said detecting comprises liquid chromatography-mass spectrometry (LC-MS), nuclear magnetic resonance (NMR), gas chromatography-mass spectrometry (GC-MS), capillary electrophoresis-mass spectrometry (CE-MS), Fourier transform infrared spectroscopy (FTIR), or combinations thereof.

Embodiment 5. A method for determining whether or not a mammal is likely to develop a radiation injury following radiation exposure, wherein said method comprises:
(a) detecting a presence or absence of at least one enriched metabolic pathway in a sample from said mammal;
(b) classifying said mammal as being likely to develop a radiation injury following radiation exposure if said presence of said enriched metabolic pathways is detected; and
(c) classifying said mammal as not being likely to develop a radiation injury following radiation exposure if said enriched metabolic pathways is not detected.

Embodiment 6. The method of embodiment 5, wherein said at least one enriched metabolic pathway is selected from the group consisting of Warburg effect, arginine and proline metabolism, glutamate metabolism, pyruvate metabolism, fatty acid metabolism, propanoate metabolism, nicotinate and nicotinamide metabolism, gluconeogenesis, ammonia recycling, pyrimidine metabolism, urea cycle, selenoamino acid metabolism, mitochondrial beta-oxidation of long chain saturated fatty acids, mitochondrial beta-oxidation of short chain saturated fatty acids, cysteine metabolism, oxidation of branched chain fatty acids, glycolysis, purine metabolism, carnitine synthesis, glutathione metabolism, lactose synthesis, beta oxidation of very long chain fatty acids, and valine, leucine and isoleucine degradation.

Embodiment 7. The method of any one of embodiments 1-6, wherein said mammal is a human.

Embodiment 8. The method of any one of embodiments 1-7, wherein said sample is selected from the group consisting of whole blood, serum, plasma, urine, cerebrospinal fluid (CSF), saliva, jejunum tissue, lung tissue, heart tissue, kidney tissue, skin tissue, bone marrow, gastrointestinal tract tissue, cardiovascular system tissue, central nervous system (CNS) tissue, hematopoietic cells, and a fecal sample.

Embodiment 9. The method of any one of embodiments 1-8, wherein said radiation exposure comprises radiation therapy.

Embodiment 10. The method of embodiment 9, wherein said mammal has cancer.

Embodiment 11. The method of any one of embodiments 1-8, wherein said radiation exposure occurs during an event selected from the group consisting of an industrial accident, terrorist attack, military action, commercial exposure, medical procedure, environmental exposure, and any combinations thereof.

Embodiment 12. The method of any one of embodiments 1-8, wherein said radiation exposure comprises a radiation source selected from the group consisting of ionizing radiation, non-ionizing radiation, low energy particles, and high energy particles.

Embodiment 13. The method of any one of embodiments 1-12, wherein said radiation injury comprises a cutaneous radiation injury (CRI) and/or radiation syndrome (ARS).

Embodiment 14. A method for treating a mammal having cancer, wherein said method comprises:
(a) detecting an absence of an altered level of at least five metabolites in a sample from said mammal; and
(b) administering a radiation therapy to said mammal.

Embodiment 15. The method of embodiment 14, wherein said altered level of said at least five metabolites is an increased level, and wherein said at least five metabolites are selected from the group consisting of 10,11-dihydro-12r-hydroxy-leukotriene E4, 12-oxo-20-trihydroxy-leukotriene B4, 2-amino-3-phosphonopropionic acid, 3-hydroxybutyrate, 5-hydroxyindoleacetic acid, 5-hydroxytryptophol glucuronide, 7-methylguanosine, acetyldigitoxin, alpha-CEHC, D-glucose, formamidopyrimidine nucleoside triphosphate, gamma glutamylglutamic acid, gamma-glutamylthreonine, ganglioside GA1 (d18:1/16:0), ganglioside GM2 (d18:1/18:1(11z)), L-beta-aspartyl-L-threonine, leucyl-valine, leukotriene F4, L-isoleucine, L-leucine, lysoPC(22:2(13z,16z)), N-a-acetyl-L-arginine, N-acetyltryptophan, N-gamma-L-glutamyl-D-alanine, PG(18:3(9Z,12Z,15Z)/20:3(5Z,8Z,11Z)), phenylalanylphenylalanine, phosphoribosyl-AMP, p-hydroxyphenylacetic acid, PS(14:0/14:1(9Z)), PS(20:4(5Z,8Z,11Z,14Z)/14:1(9Z)), pyruvate, tetrahexosylceramide (d18:1/16:0), trans-3-hydroxycotinine glucuronide, trimethylamine N-oxide, uridine, valine, vanylglycol, L-carnitine, tiglyl-CoA, 2,2-dimethylsuccinic acid, 3-methyl-2-oxovalerate, anserine, L-phenylalanine, L-tryptophan, N,N-Dimethylglycine, N6-acetyllysine, N-acetylysteine, propylene glycol, undecanoic acid, and xanthurenate.

Embodiment 16. The method of embodiment 14, wherein said altered level of said at least five metabolites is a decreased level, and wherein said at least five metabolites are selected from the group consisting of 1-methylhistidine, 2-aminomuconic acid semialdehyde, 2-methylguanosine, 2-oxoarginine, 2-pyrroloylglycine, 3-(3,5-diiodo-4-hydroxyphenyl)pyruvate, 3-dehydroxycarnitine, 3-hydroxyoctanoyl carnitine, 3-nitrotyrosine, 3′-O-methyladenosine, 4-guanidinobutanoic acid, 7-methylguanine, alanyl-serine, allantoic acid, argininic acid, arginyl-methionine, citric acid, creatine, cysteic acid, cysteinyl-cysteine, DL-2-aminooctanoic acid, dUMP, gamma-glutamyltyrosine, glutaminyl-lysine, glutarylcarnitine, glycerophosphocholine, glyceryl lactopalmitate, hippuric acid, IDP, L-acetylcarnitine, L-alanine, L-aspartyl-4-phosphate, L-carnitine, L-glutamine, L-histidine, L-isoleucine, L-phenylalanine, L-tyrosine, melatonin, N1-methyl-2-pyridone-5-carboxamide, N6,N6,N6-trimethyl-L-lysine, N-acetyl-L-methionine, nicotine imine, N-ribosylhistidine, O-isobutyryl-L-carnitine, phenol sulphate, phenylpyruvic acid, picolinoylglycine, proline betaine, pyrogallol-2-O-glucuronide, riboflavin, serinyl-hydroxyproline, sphinganine 1-phosphate, trans-3-coumarate, tyrosyl-glutamate, urocanic acid, 1H-indole-3-carboxaldehyde, 2-hydroxyadenine, allodesmosine, arginyl-histidine, guanine, hypoxanthine, LysoPC(14:0), LysoPC(15:0), LysoPC(18:3(6Z,9Z,12Z)), LysoPE(0:0/18:1(11Z)), phosphoric acid, pyrrolidine, uridine, 1,9-dimethylurate, 2-(3-carboxy-3-aminopropyl)-L-histidine, adenosine diphosphate ribose, adenosine thiamine triphosphate, ADP, ATP, cholesterol glucuronide, D-galactose, D-glucose, GTP, malonyl-camitin, N-acetyl-D-glucosamine, NAD⁺, NADH, NADP⁺, N-decanoylglycine, phosphoribosyl-AMP, purine, and tiglylcarnitine.

Embodiment 17. The method of any one of embodiments 14-16, wherein said detecting comprises LC-MS, NMR, GC-MS, CE-MS, FTIR, or combinations thereof.

Embodiment 18. A method for treating a mammal having cancer, wherein said method comprises:
(a) detecting an absence of at least one enriched metabolic pathway in a sample from said mammal; and
(b) administering a radiation therapy to said mammal.

Embodiment 19. The method of embodiment 18, wherein said at least one enriched metabolic pathway is selected from the group consisting of Warburg effect, arginine and proline metabolism, glutamate metabolism, pyruvate metabolism, fatty acid metabolism, propanoate metabolism, nicotinate and nicotinamide metabolism, gluconeogenesis, ammonia recycling, pyrimidine metabolism, urea cycle, selenoamino acid metabolism, mitochondrial beta-oxidation of long chain saturated fatty acids, mitochondrial beta-oxidation of short chain saturated fatty acids, cysteine metabolism, oxidation of branched chain fatty acids, glycolysis, purine metabolism, carnitine synthesis, glutathione metabolism, lactose synthesis, beta oxidation of very long chain fatty acids, and valine, leucine and isoleucine degradation.

Embodiment 20. The method of any one of embodiments 14-19, wherein said mammal is a human.

Embodiment 21. The method of any one of embodiments 14-20, wherein said sample is selected from the group consisting of whole blood, serum, plasma, urine, CSF, saliva, jejunum tissue, lung tissue, heart tissue, kidney tissue, skin tissue, bone marrow, gastrointestinal tract tissue, cardiovascular system tissue, CNS tissue, hematopoietic cells, and a fecal sample.

Embodiment 22. A method for treating a mammal having cancer, wherein said method comprises:
(a) detecting a presence of an altered level of at least five metabolites in a sample from said mammal; and
(b) administering a cancer treatment to said mammal, wherein said cancer treatment is not a radiation therapy.

Embodiment 23. The method of claim 22, wherein said altered level of said at least five metabolites is an increased level, and wherein said at least five metabolites are selected from the group consisting of 10,11-dihydro-12r-hydroxy-leukotriene E4, 12-oxo-20-trihydroxy-leukotriene B4, 2-amino-3-phosphonopropionic acid, 3-hydroxybutyrate, 5-hydroxyindoleacetic acid, 5-hydroxytryptophol glucuronide, 7-methylguanosine, acetyldigitoxin, alpha-CEHC, D-glucose, formamidopyrimidine nucleoside triphosphate, gamma glutamylglutamic acid, gamma-glutamylthreonine, ganglioside GA1 (d18:1/16:0), ganglioside GM2 (d18:1/18:1(11z)), L-beta-aspartyl-L-threonine, leucyl-valine, leukotriene F4, L-isoleucine, L-leucine, lysoPC(22:2(13z,16z)), N-a-acetyl-L-arginine, N-acetyltryptophan, N-gamma-L-glutamyl-D-alanine, PG(18:3(9Z,12Z,15Z)/20:3(5Z,8Z,11Z)), phenylalanylphenylalanine, phosphoribosyl-AMP, p-hydroxyphenylacetic acid, PS(14:0/14:1(9Z)), PS(20:4(5Z,8Z,11Z,14Z)/14:1(9Z)), pyruvate, tetrahexosylceramide (d18:1/16:0), trans-3-hydroxycotinine glucuronide, trimethylamine N-oxide, uridine, valine, vanylglycol, L-carnitine, tiglyl-CoA, 2,2-dimethylsuccinic acid, 3-methyl-2-oxovalerate, anserine, L-phenylalanine, L-tryptophan, N,N-Dimethylglycine, N6-acetyllysine, N-acetylysteine, propylene glycol, undecanoic acid, and xanthurenate.

Embodiment 24. The method of embodiment 22, wherein said altered level of said at least five metabolites is a decreased level, and wherein said at least five metabolites are selected from the group consisting of 1-methylhistidine, 2-aminomuconic acid semialdehyde, 2-methylguanosine, 2-oxoarginine, 2-pyrroloylglycine, 3-(3,5-diiodo-4-hydroxyphenyl)pyruvate, 3-dehydroxycarnitine, 3-hydroxyoctanoyl carnitine, 3-nitrotyrosine, 3′-O-methyladenosine, 4-guanidinobutanoic acid, 7-methylguanine, alanyl-serine, allantoic acid, argininic acid, arginyl-methionine, citric acid, creatine, cysteic acid, cysteinyl-cysteine, DL-2-aminooctanoic acid, dUMP, gamma-glutamyltyrosine, glutaminyl-lysine, glutarylcarnitine, glycerophosphocholine, glyceryl lactopalmitate, hippuric acid, IDP, L-acetylcarnitine, L-alanine, L-aspartyl-4-phosphate, L-carnitine, L-glutamine, L-histidine, L-isoleucine, L-phenylalanine, L-tyrosine, melatonin, N1-methyl-2-pyridone-5-carboxamide, N6,N6,N6-trimethyl-L-lysine, N-acetyl-L-methionine, nicotine imine, N-ribosylhistidine, O-isobutyryl-L-carnitine, phenol sulphate, phenylpyruvic acid, picolinoylglycine, proline betaine, pyrogallol-2-O-glucuronide, riboflavin, serinyl-hydroxyproline, sphinganine 1-phosphate, trans-3-coumarate, tyrosyl-glutamate, urocanic acid, 1H-indole-3-carboxaldehyde, 2-hydroxyadenine, allodesmosine, arginyl-histidine, guanine, hypoxanthine, LysoPC(14:0), LysoPC(15:0), LysoPC(18:3(6Z,9Z,12Z)), LysoPE(0:0/18:1(11Z)), phosphoric acid, pyrrolidine, uridine, 1,9-dimethylurate, 2-(3-carboxy-3-aminopropyl)-L-histidine, adenosine diphosphate ribose, adenosine thiamine triphosphate, ADP, ATP, cholesterol glucuronide, D-galactose, D-glucose, GTP, malonyl-camitin, N-acetyl-D-glucosamine, NAD⁺, NADH, NADP⁺, N-decanoylglycine, phosphoribosyl-AMP, purine, and tiglylcarnitine.

Embodiment 25. The method of any one of embodiments 22-24, wherein said detecting comprises LC-MS, NMR, GC-MS, CE-MS, FTIR, or combinations thereof.

Embodiment 26. A method for treating a mammal having cancer, wherein said method comprises:
(a) detecting a presence of at least one enriched metabolic pathway in a sample from said mammal; and
(b) administering a cancer treatment to said mammal, wherein said cancer treatment is not a radiation therapy.

Embodiment 27. The method of embodiment 26, wherein said at least one enriched metabolic pathway is selected from the group consisting of Warburg effect, arginine and proline metabolism, glutamate metabolism, pyruvate metabolism, fatty acid metabolism, propanoate metabolism, nicotinate and nicotinamide metabolism, gluconeogenesis, ammonia recycling, pyrimidine metabolism, urea cycle, selenoamino acid metabolism, mitochondrial beta-oxidation of long chain saturated fatty acids, mitochondrial beta-oxidation of short chain saturated fatty acids, cysteine metabolism, oxidation of branched chain fatty acids, glycolysis, purine metabolism, carnitine synthesis, glutathione metabolism, lactose synthesis, beta oxidation of very long chain fatty acids, and valine, leucine and isoleucine degradation.

Embodiment 28. The method of any one of embodiments 22-27, wherein said mammal is a human.

Embodiment 29. The method of any one of embodiments 22-28, wherein said sample is selected from the group consisting of whole blood, serum, plasma, urine, CSF, saliva, jejunum tissue, lung tissue, heart tissue, kidney tissue, skin tissue, bone marrow, gastrointestinal tract tissue, cardiovascular system tissue, CNS tissue, hematopoietic cells, and a fecal sample.

Embodiment 30. The method of any one of embodiments 22-29, wherein said cancer treatment comprises administering an anti-cancer agent selected from the group consisting of a chemotherapeutic agent, a targeted cancer drug, an immunotherapy drug, and a hormone therapy drug.

Embodiment 31. The method of any one of embodiments 22-29, wherein said cancer treatment comprises surgery.

Embodiment 32. A method for treating a mammal, wherein said method comprises:
(a) identifying said mammal as being likely to develop a radiation injury following radiation exposure by detecting a presence of an altered level of at least five metabolites in a sample from said mammal; and
(b) administering a radioprotective agent or radio-mitigation agent to said mammal.

Embodiment 33. The method of embodiment 32, wherein said altered level of said at least five metabolites is an increased level, and wherein said at least five metabolites are selected from the group consisting of 10,11-dihydro-12r-hydroxy-leukotriene E4, 12-oxo-20-trihydroxy-leukotriene B4, 2-amino-3-phosphonopropionic acid, 3-hydroxybutyrate, 5-hydroxyindoleacetic acid, 5-hydroxytryptophol glucuronide, 7-methylguanosine, acetyldigitoxin, alpha-CEHC, D-glucose, formamidopyrimidine nucleoside triphosphate, gamma glutamylglutamic acid, gamma-glutamylthreonine, ganglioside GA1 (d18:1/16:0), ganglioside GM2 (d18:1/18:1(11z)), L-beta-aspartyl-L-threonine, leucyl-valine, leukotriene F4, L-isoleucine, L-leucine, lysoPC(22:2(13z,16z)), N-a-acetyl-L-arginine, N-acetyltryptophan, N-gamma-L-glutamyl-D-alanine, PG(18:3(9Z,12Z,15Z)/20:3(5Z,8Z,11Z)), phenylalanylphenylalanine, phosphoribosyl-AMP, p-hydroxyphenylacetic acid, PS(14:0/14:1(9Z)), PS(20:4(5Z,8Z,11Z,14Z)/14:1(9Z)), pyruvate, tetrahexosylceramide (d18:1/16:0), trans-3-hydroxycotinine glucuronide, trimethylamine N-oxide, uridine, valine, vanylglycol, L-carnitine, tiglyl-CoA, 2,2-dimethylsuccinic acid, 3-methyl-2-oxovalerate, anserine, L-phenylalanine, L-tryptophan, N,N-Dimethylglycine, N6-acetyllysine, N-acetylysteine, propylene glycol, undecanoic acid, and xanthurenate.

Embodiment 34. The method of embodiment 32, wherein said altered level of said at least five metabolites is a decreased level, and wherein said at least five metabolites are selected from the group consisting of 1-methylhistidine, 2-aminomuconic acid semialdehyde, 2-methylguanosine, 2-oxoarginine, 2-pyrroloylglycine, 3-(3,5-diiodo-4-hydroxyphenyl)pyruvate, 3-dehydroxycarnitine, 3-hydroxyoctanoyl carnitine, 3-nitrotyrosine, 3′-O-methyladenosine, 4-guanidinobutanoic acid, 7-methylguanine, alanyl-serine, allantoic acid, argininic acid, arginyl-methionine, citric acid, creatine, cysteic acid, cysteinyl-cysteine, DL-2-aminooctanoic acid, dUMP, gamma-glutamyltyrosine, glutaminyl-lysine, glutarylcarnitine, glycerophosphocholine, glyceryl lactopalmitate, hippuric acid, IDP, L-acetylcarnitine, L-alanine, L-aspartyl-4-phosphate, L-carnitine, L-glutamine, L-histidine, L-isoleucine, L-phenylalanine, L-tyrosine, melatonin, N1-methyl-2-pyridone-5-carboxamide, N6,N6,N6-trimethyl-L-lysine, N-acetyl-L-methionine, nicotine imine, N-ribosylhistidine, O-isobutyryl-L-carnitine, phenol sulphate, phenylpyruvic acid, picolinoylglycine, proline betaine, pyrogallol-2-O-glucuronide, riboflavin, serinyl-hydroxyproline, sphinganine 1-phosphate, trans-3-coumarate, tyrosyl-glutamate, urocanic acid, 1H-indole-3-carboxaldehyde, 2-hydroxyadenine, allodesmosine, arginyl-histidine, guanine, hypoxanthine, LysoPC(14:0), LysoPC(15:0), LysoPC(18:3(6Z,9Z,12Z)), LysoPE(0:0/18:1(11Z)), phosphoric acid, pyrrolidine, uridine, 1,9-dimethylurate, 2-(3-carboxy-3-aminopropyl)-L-histidine, adenosine diphosphate ribose, adenosine thiamine triphosphate, ADP, ATP, cholesterol glucuronide, D-galactose, D-glucose, GTP, malonyl-camitin, N-acetyl-D-glucosamine, NAD⁺, NADH, NADP⁺, N-decanoylglycine, phosphoribosyl-AMP, purine, and tiglylcarnitine.

Embodiment 35. The method of any one of embodiments 32-34, wherein said detecting comprises LC-MS, NMR, GC-MS, CE-MS, FTIR, or combinations thereof.

Embodiment 36. A method for treating a mammal, wherein said method comprises:
(a) identifying said mammal as being likely to develop a radiation injury following radiation exposure by detecting a presence of at least one enriched metabolic pathway in a sample from said mammal; and
(b) administering a radioprotective agent or a radio-mitigation agent to said mammal.

Embodiment 37. The method of embodiment 36, wherein said at least one enriched metabolic pathway is selected from the group consisting of Warburg effect, arginine and proline metabolism, glutamate metabolism, pyruvate metabolism, fatty acid metabolism, propanoate metabolism, nicotinate and nicotinamide metabolism, gluconeogenesis, ammonia recycling, pyrimidine metabolism, urea cycle, selenoamino acid metabolism, mitochondrial beta-oxidation of long chain saturated fatty acids, mitochondrial beta-oxidation of short chain saturated fatty acids, cysteine metabolism, oxidation of branched chain fatty acids, glycolysis, purine metabolism, carnitine synthesis, glutathione metabolism, lactose synthesis, beta oxidation of very long chain fatty acids, and valine, leucine and isoleucine degradation.

Embodiment 38. The method of any one of embodiments 32-37, wherein said mammal is a human.

Embodiment 39. The method of any one of embodiments 32-38, wherein said sample is selected from the group consisting of whole blood, serum, plasma, urine, CSF, saliva, jejunum tissue, lung tissue, heart tissue, kidney tissue, skin tissue, bone marrow, gastrointestinal tract tissue, cardiovascular system tissue, CNS tissue, hematopoietic cells, and a fecal sample.

Embodiment 40. The method of any one of embodiments 32-39, wherein said radioprotective agent is selected from the group consisting of amifostine (2-(3-aminpropyl) aminoethylphosphorothioate), potassium iodide (KI), Prussian blue, diethylenetriamine pentaacetate (DTPA), and filgrastim.

Embodiment 41. The method of any one of embodiments 32-40, wherein said radiation exposure comprises radiation therapy.

Embodiment 42. The method of embodiment 41, wherein said mammal has cancer.

Embodiment 43. The method of any one of embodiments 32-40, wherein said radiation exposure occurs during an event selected from the group consisting of an industrial accident, terrorist attack, military action, commercial exposure, medical procedure, environmental exposure, and any combinations thereof.

Embodiment 44. The method of any one of embodiments 32-40, wherein said radiation exposure comprises a radiation source selected from the group consisting of ionizing radiation, non-ionizing radiation, low energy particles, and high energy particles.

Embodiment 45. The method of any one of embodiments 32-44, wherein said radiation injury comprises a cutaneous radiation injury (CRI) and/or radiation syndrome (ARS).

Embodiment 48. A method for monitoring a mammal for radiation injury following radiation exposure, wherein said method comprises:
(a) detecting a presence or absence of an altered level of at least five metabolites in a sample from said mammal;
(b) classifying said mammal as being likely to develop a radiation injury following radiation exposure if said presence of said altered level is detected; and
(c) classifying said mammal as not being likely to develop a radiation injury if said presence of said altered level is not detected.

Embodiment 49. The method of embodiment 48, wherein said altered level of said at least five metabolites is an increased level, and wherein said at least five metabolites are selected from the group consisting of 10,11-dihydro-12r-hydroxy-leukotriene E4, 12-oxo-20-trihydroxy-leukotriene B4, 2-amino-3-phosphonopropionic acid, 3-hydroxybutyrate, 5-hydroxyindoleacetic acid, 5-hydroxytryptophol glucuronide, 7-methylguanosine, acetyldigitoxin, alpha-CEHC, D-glucose, formamidopyrimidine nucleoside triphosphate, gamma glutamylglutamic acid, gamma-glutamylthreonine, ganglioside GA1 (d18:1/16:0), ganglioside GM2 (d18:1/18:1(11z)), L-beta-aspartyl-L-threonine, leucyl-valine, leukotriene F4, L-isoleucine, L-leucine, lysoPC(22:2(13z,16z)), N-a-acetyl-L-arginine, N-acetyltryptophan, N-gamma-L-glutamyl-D-alanine, PG(18:3(9Z,12Z,15Z)/20:3(5Z,8Z,11Z)), phenylalanylphenylalanine, phosphoribosyl-AMP, p-hydroxyphenylacetic acid, PS(14:0/14:1(9Z)), PS(20:4(5Z,8Z,11Z,14Z)/14:1(9Z)), pyruvate, tetrahexosylceramide (d18:1/16:0), trans-3-hydroxycotinine glucuronide, trimethylamine N-oxide, uridine, valine, vanylglycol, L-carnitine, tiglyl-CoA, 2,2-dimethylsuccinic acid, 3-methyl-2-oxovalerate, anserine, L-phenylalanine, L-tryptophan, N,N-Dimethylglycine, N6-acetyllysine, N-acetylysteine, propylene glycol, undecanoic acid, and xanthurenate.

Embodiment 50. The method of embodiment 48, wherein said altered level of said at least five metabolites is a decreased level, and wherein said at least five metabolites are selected from the group consisting of 1-methylhistidine, 2-aminomuconic acid semialdehyde, 2-methylguanosine, 2-oxoarginine, 2-pyrroloylglycine, 3-(3,5-diiodo-4-hydroxyphenyl)pyruvate, 3-dehydroxycarnitine, 3-hydroxyoctanoyl carnitine, 3-nitrotyrosine, 3′-O-methyladenosine, 4-guanidinobutanoic acid, 7-methylguanine, alanyl-serine, allantoic acid, argininic acid, arginyl-methionine, citric acid, creatine, cysteic acid, cysteinyl-cysteine, DL-2-aminooctanoic acid, dUMP, gamma-glutamyltyrosine, glutaminyl-lysine, glutarylcarnitine, glycerophosphocholine, glyceryl lactopalmitate, hippuric acid, IDP, L-acetylcarnitine, L-alanine, L-aspartyl-4-phosphate, L-carnitine, L-glutamine, L-histidine, L-isoleucine, L-phenylalanine, L-tyrosine, melatonin, N1-methyl-2-pyridone-5-carboxamide, N6,N6,N6-trimethyl-L-lysine, N-acetyl-L-methionine, nicotine imine, N-ribosylhistidine, O-isobutyryl-L-carnitine, phenol sulphate, phenylpyruvic acid, picolinoylglycine, proline betaine, pyrogallol-2-O-glucuronide, riboflavin, serinyl-hydroxyproline, sphinganine 1-phosphate, trans-3-coumarate, tyrosyl-glutamate, urocanic acid, 1H-indole-3-carboxaldehyde, 2-hydroxyadenine, allodesmosine, arginyl-histidine, guanine, hypoxanthine, LysoPC(14:0), LysoPC(15:0), LysoPC(18:3(6Z,9Z,12Z)), LysoPE(0:0/18:1(11Z)), phosphoric acid, pyrrolidine, uridine, 1,9-dimethylurate, 2-(3-carboxy-3-aminopropyl)-L-histidine, adenosine diphosphate ribose, adenosine thiamine triphosphate, ADP, ATP, cholesterol glucuronide, D-galactose, D-glucose, GTP, malonyl-camitin, N-acetyl-D-glucosamine, NAD⁺, NADH, NADP⁺, N-decanoylglycine, phosphoribosyl-AMP, purine, and tiglylcarnitine.

Embodiment 52. The method of any one of embodiments 48-50, wherein said detecting comprises LC-MS, NMR, GC-MS, CE-MS, FTIR, or combinations thereof.

Embodiment 53. A method for monitoring a mammal for radiation injury following radiation exposure, wherein said method comprises:
(a) detecting a presence or absence of at least one enriched metabolic pathway in a sample from said mammal;
(b) classifying said mammal as being likely to develop a radiation injury following radiation exposure if said presence of said enriched metabolic pathways is detected; and
(c) classifying said mammal as not being likely to develop a radiation injury if said presence of said enriched metabolic pathways is not detected.

Embodiment 54. The method of embodiment 53, wherein said at least one enriched metabolic pathway is selected from the group consisting of Warburg effect, arginine and proline metabolism, glutamate metabolism, pyruvate metabolism, fatty acid metabolism, propanoate metabolism, nicotinate and nicotinamide metabolism, gluconeogenesis, ammonia recycling, pyrimidine metabolism, urea cycle, selenoamino acid metabolism, mitochondrial beta-oxidation of long chain saturated fatty acids, mitochondrial beta-oxidation of short chain saturated fatty acids, cysteine metabolism, oxidation of branched chain fatty acids, glycolysis, purine metabolism, carnitine synthesis, glutathione metabolism, lactose synthesis, beta oxidation of very long chain fatty acids, and valine, leucine and isoleucine degradation.

Embodiment 55. The method of any one of embodiments 48-54, wherein said mammal is a human.

Embodiment 56. The method of any one of embodiments 48-55, wherein said sample is selected from the group consisting of whole blood, serum, plasma, urine, CSF, saliva, jejunum tissue, lung tissue, heart tissue, kidney tissue, skin tissue, bone marrow, gastrointestinal tract tissue, cardiovascular system tissue, CNS tissue, hematopoietic cells, and a fecal sample.

Embodiment 57. The method of any one of embodiments 48-56, wherein said radiation exposure comprises radiation therapy.

Embodiment 58. The method of embodiment 54, wherein said mammal has cancer.

Embodiment 59. The method of any one of embodiments 48-56, wherein said radiation exposure occurs during an event selected from the group consisting of an industrial accident, terrorist attack, military action, commercial exposure, medical procedure, environmental exposure, and any combinations thereof.

Embodiment 60. The method of any one of embodiments 48-56, wherein said radiation exposure comprises a radiation source selected from the group consisting of ionizing radiation, non-ionizing radiation, low energy particles, and high energy particles.

Embodiment 61. The method of any one of embodiments 48-60, wherein said radiation injury comprises a cutaneous radiation injury (CRI) and/or radiation syndrome (ARS).

Embodiment 62. A method for identifying a radioprotective agent, said method comprising:
(a) subjecting a non-human mammal to radiation exposure;
(b) administering a candidate compound to said non-human mammal;
(c) detecting a presence or absence of an altered level of at least five metabolites in a sample from said non-human mammal;
(d) not classifying said candidate compound as a radioprotective agent if said presence of said altered level is detected; and
(e) classifying said candidate compound as a radioprotective agent if said presence of said altered level is not detected.

Embodiment 63. The method of embodiment 62, wherein said altered level of said at least five metabolites is an increased level, and wherein said at least five metabolites are selected from the group consisting of 10,11-dihydro-12r-hydroxy-leukotriene E4, 12-oxo-20-trihydroxy-leukotriene B4, 2-amino-3-phosphonopropionic acid, 3-hydroxybutyrate, 5-hydroxyindoleacetic acid, 5-hydroxytryptophol glucuronide, 7-methylguanosine, acetyldigitoxin, alpha-CEHC, D-glucose, formamidopyrimidine nucleoside triphosphate, gamma glutamylglutamic acid, gamma-glutamylthreonine, ganglioside GA1 (d18:1/16:0), ganglioside GM2 (d18:1/18:1(11z)), L-beta-aspartyl-L-threonine, leucyl-valine, leukotriene F4, L-isoleucine, L-leucine, lysoPC(22:2(13z,16z)), N-a-acetyl-L-arginine, N-acetyltryptophan, N-gamma-L-glutamyl-D-alanine, PG(18:3(9Z,12Z,15Z)/20:3(5Z,8Z,11Z)), phenylalanylphenylalanine, phosphoribosyl-AMP, p-hydroxyphenylacetic acid, PS(14:0/14:1(9Z)), PS(20:4(5Z,8Z,11Z,14Z)/14:1(9Z)), pyruvate, tetrahexosylceramide (d18:1/16:0), trans-3-hydroxycotinine glucuronide, trimethylamine N-oxide, uridine, valine, vanylglycol, L-carnitine, tiglyl-CoA, 2,2-dimethylsuccinic acid, 3-methyl-2-oxovalerate, anserine, L-phenylalanine, L-tryptophan, N,N-Dimethylglycine, N6-acetyllysine, N-acetylysteine, propylene glycol, undecanoic acid, and xanthurenate.

Embodiment 64. The method of embodiment 62, wherein said altered level of said at least five metabolites is a decreased level, and wherein said at least five metabolites are selected from the group consisting of 1-methylhistidine, 2-aminomuconic acid semialdehyde, 2-methylguanosine, 2-oxoarginine, 2-pyrroloylglycine, 3-(3,5-diiodo-4-hydroxyphenyl)pyruvate, 3-dehydroxycarnitine, 3-hydroxyoctanoyl carnitine, 3-nitrotyrosine, 3′-O-methyladenosine, 4-guanidinobutanoic acid, 7-methylguanine, alanyl-serine, allantoic acid, argininic acid, arginyl-methionine, citric acid, creatine, cysteic acid, cysteinyl-cysteine, DL-2-aminooctanoic acid, dUMP, gamma-glutamyltyrosine, glutaminyl-lysine, glutarylcarnitine, glycerophosphocholine, glyceryl lactopalmitate, hippuric acid, IDP, L-acetylcarnitine, L-alanine, L-aspartyl-4-phosphate, L-carnitine, L-glutamine, L-histidine, L-isoleucine, L-phenylalanine, L-tyrosine, melatonin, N1-methyl-2-pyridone-5-carboxamide, N6,N6,N6-trimethyl-L-lysine, N-acetyl-L-methionine, nicotine imine, N-ribosylhistidine, O-isobutyryl-L-carnitine, phenol sulphate, phenylpyruvic acid, picolinoylglycine, proline betaine, pyrogallol-2-O-glucuronide, riboflavin, serinyl-hydroxyproline, sphinganine 1-phosphate, trans-3-coumarate, tyrosyl-glutamate, urocanic acid, 1H-indole-3-carboxaldehyde, 2-hydroxyadenine, allodesmosine, arginyl-histidine, guanine, hypoxanthine, LysoPC(14:0), LysoPC(15:0), LysoPC(18:3(6Z,9Z,12Z)), LysoPE(0:0/18:1(11Z)), phosphoric acid, pyrrolidine, uridine, 1,9-dimethylurate, 2-(3-carboxy-3-aminopropyl)-L-histidine, adenosine diphosphate ribose, adenosine thiamine triphosphate, ADP, ATP, cholesterol glucuronide, D-galactose, D-glucose, GTP, malonyl-camitin, N-acetyl-D-glucosamine, NAD⁺, NADH, NADP⁺, N-decanoylglycine, phosphoribosyl-AMP, purine, and tiglylcarnitine.

Embodiment 65. The method of any one of embodiments 62-64, wherein said detecting comprises LC-MS, NMR, GC-MS, CE-MS, FTIR, or combinations thereof.

Embodiment 66. A method for identifying a radioprotective agent, said method comprising:
(a) subjecting a non-human mammal to radiation exposure;
(b) administering a candidate compound to said non-human mammal;
(c) detecting a presence or absence of at least one enriched metabolic pathway in a sample from said non-human mammal;
(d) not classifying said candidate compound as a radioprotective agent if said presence of said enriched metabolic pathways is detected; and
(e) classifying said candidate compound as a radioprotective agent if said presence of said enriched metabolic pathways is not detected.

Embodiment 67. The method of embodiment 66, wherein said at least one enriched metabolic pathway is selected from the group consisting of Warburg effect, arginine and proline metabolism, glutamate metabolism, pyruvate metabolism, fatty acid metabolism, propanoate metabolism, nicotinate and nicotinamide metabolism, gluconeogenesis, ammonia recycling, pyrimidine metabolism, urea cycle, selenoamino acid metabolism, mitochondrial beta-oxidation of long chain saturated fatty acids, mitochondrial beta-oxidation of short chain saturated fatty acids, cysteine metabolism, oxidation of branched chain fatty acids, glycolysis, purine metabolism, carnitine synthesis, glutathione metabolism, lactose synthesis, beta oxidation of very long chain fatty acids, and valine, leucine and isoleucine degradation.

Embodiment 68. The method of any one of embodiments 62-67, wherein said non-human mammal is a non-human primate.

Embodiment 69. The method of any one of embodiments 62-68, wherein said sample is selected from the group consisting of whole blood, serum, plasma, urine, CSF, saliva, jejunum tissue, lung tissue, heart tissue, kidney tissue, skin tissue, bone marrow, gastrointestinal tract tissue, cardiovascular system tissue, CNS tissue, hematopoietic cells, and a fecal sample.

Embodiment 70. The method of any one of embodiments 62-69, wherein said radiation exposure occurs during an event selected from the group consisting of an industrial accident, terrorist attack, military action, commercial exposure, medical procedure, environmental exposure, and any combinations thereof.

Embodiment 71. The method of any one of embodiments 62-69, wherein said radiation exposure comprises a radiation source selected from the group consisting of ionizing radiation, non-ionizing radiation, low energy particles, and high energy particles.

Embodiment 72. A method for monitoring a mammal having been administered a radiation counter measure following radiation exposure, wherein said method comprises:
(a) detecting a presence or absence of an altered level of at least five metabolites in a sample from said mammal;
(b) classifying said radiation counter measure as being an effective treatment for said mammal if said presence of said altered level is not detected; and
(c) classifying said radiation counter measure as not being an effective treatment for said mammal if said presence of said altered level is detected.

Embodiment 73. The method of embodiment 72, wherein said altered level of said at least five metabolites is an increased level, and wherein said at least five metabolites are selected from the group consisting of 10,11-dihydro-12r-hydroxy-leukotriene E4, 12-oxo-20-trihydroxy-leukotriene B4, 2-amino-3-phosphonopropionic acid, 3-hydroxybutyrate, 5-hydroxyindoleacetic acid, 5-hydroxytryptophol glucuronide, 7-methylguanosine, acetyldigitoxin, alpha-CEHC, D-glucose, formamidopyrimidine nucleoside triphosphate, gamma glutamylglutamic acid, gamma-glutamylthreonine, ganglioside GA1 (d18:1/16:0), ganglioside GM2 (d18:1/18:1(11z)), L-beta-aspartyl-L-threonine, leucyl-valine, leukotriene F4, L-isoleucine, L-leucine, lysoPC(22:2(13z,16z)), N-a-acetyl-L-arginine, N-acetyltryptophan, N-gamma-L-glutamyl-D-alanine, PG(18:3(9Z,12Z,15Z)/20:3(5Z,8Z,11Z)), phenylalanylphenylalanine, phosphoribosyl-AMP, p-hydroxyphenylacetic acid, PS(14:0/14:1(9Z)), PS(20:4(5Z,8Z,11Z,14Z)/14:1(9Z)), pyruvate, tetrahexosylceramide (d18:1/16:0), trans-3-hydroxycotinine glucuronide, trimethylamine N-oxide, uridine, valine, vanylglycol, L-carnitine, tiglyl-CoA, 2,2-dimethylsuccinic acid, 3-methyl-2-oxovalerate, anserine, L-phenylalanine, L-tryptophan, N,N-Dimethylglycine, N6-acetyllysine, N-acetylysteine, propylene glycol, undecanoic acid, and xanthurenate.

Embodiment 74. The method of embodiment 72, wherein said altered level of said at least five metabolites is a decreased level, and wherein said at least five metabolites are selected from the group consisting of 1-methylhistidine, 2-aminomuconic acid semialdehyde, 2-methylguanosine, 2-oxoarginine, 2-pyrroloylglycine, 3-(3,5-diiodo-4-hydroxyphenyl)pyruvate, 3-dehydroxycarnitine, 3-hydroxyoctanoyl carnitine, 3-nitrotyrosine, 3′-O-methyladenosine, 4-guanidinobutanoic acid, 7-methylguanine, alanyl-serine, allantoic acid, argininic acid, arginyl-methionine, citric acid, creatine, cysteic acid, cysteinyl-cysteine, DL-2-aminooctanoic acid, dUMP, gamma-glutamyltyrosine, glutaminyl-lysine, glutarylcarnitine, glycerophosphocholine, glyceryl lactopalmitate, hippuric acid, IDP, L-acetylcarnitine, L-alanine, L-aspartyl-4-phosphate, L-carnitine, L-glutamine, L-histidine, L-isoleucine, L-phenylalanine, L-tyrosine, melatonin, N1-methyl-2-pyridone-5-carboxamide, N6,N6,N6-trimethyl-L-lysine, N-acetyl-L-methionine, nicotine imine, N-ribosylhistidine, O-isobutyryl-L-carnitine, phenol sulphate, phenylpyruvic acid, picolinoylglycine, proline betaine, pyrogallol-2-O-glucuronide, riboflavin, serinyl-hydroxyproline, sphinganine 1-phosphate, trans-3-coumarate, tyrosyl-glutamate, urocanic acid, 1H-indole-3-carboxaldehyde, 2-hydroxyadenine, allodesmosine, arginyl-histidine, guanine, hypoxanthine, LysoPC(14:0), LysoPC(15:0), LysoPC(18:3(6Z,9Z,12Z)), LysoPE(0:0/18:1(11Z)), phosphoric acid, pyrrolidine, uridine, 1,9-dimethylurate, 2-(3-carboxy-3-aminopropyl)-L-histidine, adenosine diphosphate ribose, adenosine thiamine triphosphate, ADP, ATP, cholesterol glucuronide, D-galactose, D-glucose, GTP, malonyl-camitin, N-acetyl-D-glucosamine, NAD⁺, NADH, NADP⁺, N-decanoylglycine, phosphoribosyl-AMP, purine, and tiglylcarnitine.

Embodiment 75. The method of any one of embodiments 72-74, wherein said detecting comprises LC-MS, NMR, GC-MS, CE-MS, FTIR, or combinations thereof.

Embodiment 76. A method for monitoring a mammal having been administered a radiation counter measure following radiation exposure, wherein said method comprises:
(a) detecting a presence or absence of at least one enriched metabolic pathway in a sample from said mammal;
(b) classifying said radiation counter measure as being an effective treatment for said mammal if said presence of said enriched metabolic pathways is not detected; and
(c) classifying said radiation counter measure as not being an effective treatment for said mammal if said presence of said enriched metabolic pathways is detected.

Embodiment 77. The method of embodiment 76, wherein said at least one enriched metabolic pathway is selected from the group consisting of Warburg effect, arginine and proline metabolism, glutamate metabolism, pyruvate metabolism, fatty acid metabolism, propanoate metabolism, nicotinate and nicotinamide metabolism, gluconeogenesis, ammonia recycling, pyrimidine metabolism, urea cycle, selenoamino acid metabolism, mitochondrial beta-oxidation of long chain saturated fatty acids, mitochondrial beta-oxidation of short chain saturated fatty acids, cysteine metabolism, oxidation of branched chain fatty acids, glycolysis, purine metabolism, carnitine synthesis, glutathione metabolism, lactose synthesis, beta oxidation of very long chain fatty acids, and valine, leucine and isoleucine degradation.

Embodiment 78. The method of any one of embodiments 72-77, wherein said mammal is a human.

Embodiment 79. The method of any one of embodiments 72-78, wherein said sample is selected from the group consisting of whole blood, serum, plasma, urine, CSF, saliva, jejunum tissue, lung tissue, heart tissue, kidney tissue, skin tissue, bone marrow, gastrointestinal tract tissue, cardiovascular system tissue, CNS tissue, hematopoietic cells, and a fecal sample.

Embodiment 80. The method of any one of embodiments 72-79, wherein said radiation exposure occurs during an event selected from the group consisting of an industrial accident, terrorist attack, military action, commercial exposure, medical procedure, environmental exposure, and any combinations thereof.

Embodiment 81. The method of any one of embodiments 72-80, wherein said radiation exposure comprises a radiation source selected from the group consisting of ionizing radiation, non-ionizing radiation, low energy particles, and high energy particles.

Embodiment 82. A method for monitoring a mammal having been administered a radiation counter measure following radiation exposure, wherein said method comprises:
(a) identifying said mammal as having been exposed to radiation;
(b) administering said radiation counter measure to said mammal;
(c) detecting a presence or absence of an altered level of at least five metabolites in a sample from said mammal;
(d) classifying said radiation counter measure as being an effective treatment for said mammal if said presence of said altered level is not detected; and
(e) classifying said radiation counter measure as not being an effective treatment for said mammal if said presence of said altered level is detected.

Embodiment 83. The method of embodiment 82, wherein said altered level of said at least five metabolites is an increased level, and wherein said at least five metabolites are selected from the group consisting of 10,11-dihydro-12r-hydroxy-leukotriene E4, 12-oxo-20-trihydroxy-leukotriene B4, 2-amino-3-phosphonopropionic acid, 3-hydroxybutyrate, 5-hydroxyindoleacetic acid, 5-hydroxytryptophol glucuronide, 7-methylguanosine, acetyldigitoxin, alpha-CEHC, D-glucose, formamidopyrimidine nucleoside triphosphate, gamma glutamylglutamic acid, gamma-glutamylthreonine, ganglioside GA1 (d18:1/16:0), ganglioside GM2 (d18:1/18:1(11z)), L-beta-aspartyl-L-threonine, leucyl-valine, leukotriene F4, L-isoleucine, L-leucine, lysoPC(22:2(13z,16z)), N-a-acetyl-L-arginine, N-acetyltryptophan, N-gamma-L-glutamyl-D-alanine, PG(18:3(9Z,12Z,15Z)/20:3(5Z,8Z,11Z)), phenylalanylphenylalanine, phosphoribosyl-AMP, p-hydroxyphenylacetic acid, PS(14:0/14:1(9Z)), PS(20:4(5Z,8Z,11Z,14Z)/14:1(9Z)), pyruvate, tetrahexosylceramide (d18:1/16:0), trans-3-hydroxycotinine glucuronide, trimethylamine N-oxide, uridine, valine, vanylglycol, L-carnitine, tiglyl-CoA, 2,2-dimethylsuccinic acid, 3-methyl-2-oxovalerate, anserine, L-phenylalanine, L-tryptophan, N,N-Dimethylglycine, N6-acetyllysine, N-acetylysteine, propylene glycol, undecanoic acid, and xanthurenate.

Embodiment 84. The method of embodiment 82, wherein said altered level of said at least five metabolites is a decreased level, and wherein said at least five metabolites are selected from the group consisting of 1-methylhistidine, 2-aminomuconic acid semialdehyde, 2-methylguanosine, 2-oxoarginine, 2-pyrroloylglycine, 3-(3,5-diiodo-4-hydroxyphenyl)pyruvate, 3-dehydroxycarnitine, 3-hydroxyoctanoyl carnitine, 3-nitrotyrosine, 3′-O-methyladenosine, 4-guanidinobutanoic acid, 7-methylguanine, alanyl-serine, allantoic acid, argininic acid, arginyl-methionine, citric acid, creatine, cysteic acid, cysteinyl-cysteine, DL-2-aminooctanoic acid, dUMP, gamma-glutamyltyrosine, glutaminyl-lysine, glutarylcarnitine, glycerophosphocholine, glyceryl lactopalmitate, hippuric acid, IDP, L-acetylcarnitine, L-alanine, L-aspartyl-4-phosphate, L-carnitine, L-glutamine, L-histidine, L-isoleucine, L-phenylalanine, L-tyrosine, melatonin, N1-methyl-2-pyridone-5-carboxamide, N6,N6,N6-trimethyl-L-lysine, N-acetyl-L-methionine, nicotine imine, N-ribosylhistidine, O-isobutyryl-L-carnitine, phenol sulphate, phenylpyruvic acid, picolinoylglycine, proline betaine, pyrogallol-2-O-glucuronide, riboflavin, serinyl-hydroxyproline, sphinganine 1-phosphate, trans-3-coumarate, tyrosyl-glutamate, urocanic acid, 1H-indole-3-carboxaldehyde, 2-hydroxyadenine, allodesmosine, arginyl-histidine, guanine, hypoxanthine, LysoPC(14:0), LysoPC(15:0), LysoPC(18:3(6Z,9Z,12Z)), LysoPE(0:0/18:1(11Z)), phosphoric acid, pyrrolidine, uridine, 1,9-dimethylurate, 2-(3-carboxy-3-aminopropyl)-L-histidine, adenosine diphosphate ribose, adenosine thiamine triphosphate, ADP, ATP, cholesterol glucuronide, D-galactose, D-glucose, GTP, malonyl-camitin, N-acetyl-D-glucosamine, NAD⁺, NADH, NADP⁺, N-decanoylglycine, phosphoribosyl-AMP, purine, and tiglylcarnitine.

Embodiment 85. The method of any one of embodiments 82-84, wherein said detecting comprises LC-MS, NMR, GC-MS, CE-MS, FTIR, or combinations thereof.

Embodiment 86. A method for monitoring a mammal having been administered a radiation counter measure following radiation exposure, wherein said method comprises:
(a) identifying said mammal as having been exposed to radiation;
(b) administering said radiation counter measure to said mammal;
(c) detecting a presence or absence of at least one enriched metabolic pathway in a sample from said mammal;
(d) classifying said radiation counter measure as being an effective treatment for said mammal if said presence of said enriched metabolic pathways is not detected; and
(e) classifying said radiation counter measure as not being an effective treatment for said mammal if said presence of said enriched metabolic pathways is detected.

Embodiment 87. The method of embodiment 86, wherein said at least one enriched metabolic pathway is selected from the group consisting of Warburg effect, arginine and proline metabolism, glutamate metabolism, pyruvate metabolism, fatty acid metabolism, propanoate metabolism, nicotinate and nicotinamide metabolism, gluconeogenesis, ammonia recycling, pyrimidine metabolism, urea cycle, selenoamino acid metabolism, mitochondrial beta-oxidation of long chain saturated fatty acids, mitochondrial beta-oxidation of short chain saturated fatty acids, cysteine metabolism, oxidation of branched chain fatty acids, glycolysis, purine metabolism, carnitine synthesis, glutathione metabolism, lactose synthesis, beta oxidation of very long chain fatty acids, and valine, leucine and isoleucine degradation.

Embodiment 88. The method of any one of embodiments 82-87, wherein said mammal is a human.

Embodiment 89. The method of any one of embodiments 82-88, wherein said sample is selected from the group consisting of whole blood, serum, plasma, urine, CSF, saliva, jejunum tissue, lung tissue, heart tissue, kidney tissue, skin tissue, bone marrow, gastrointestinal tract tissue, cardiovascular system tissue, CNS tissue, hematopoietic cells, and a fecal sample.

Embodiment 90. The method of any one of embodiments 82-89, wherein said radiation exposure occurs during an event selected from the group consisting of an industrial accident, terrorist attack, military action, commercial exposure, medical procedure, environmental exposure, and any combinations thereof.

Embodiment 91. The method of any one of embodiments 82-90, wherein said radiation exposure comprises a radiation source selected from the group consisting of ionizing radiation, non-ionizing radiation, low energy particles, and high energy particles.

Embodiment 92. A method for monitoring a mammal having been administered a radiation counter measure following radiation exposure, wherein said method comprises:
(a) detecting a presence or absence of an altered level of at least five metabolites in a sample from said mammal;
(b) classifying said radiation counter measure as being an effective treatment for said mammal if said presence of said altered level is not detected; and
(c) administering said radiation counter measure to said mammal.

Embodiment 93. A method for monitoring a mammal having been administered a first radiation counter measure following radiation exposure, wherein said method comprises:
(a) detecting a presence or absence of an altered level of at least five metabolites in a sample from said mammal;
(b) classifying said first radiation counter measure as not being an effective treatment for said mammal if said presence of said altered level is detected; and
(c) administering a second radiation counter measure to said mammal.

Embodiment 94. The method of embodiment 92 or embodiment 93, wherein said altered level of said at least five metabolites is an increased level, and wherein said at least five metabolites are selected from the group consisting of 10,11-dihydro-12r-hydroxy-leukotriene E4, 12-oxo-20-trihydroxy-leukotriene B4, 2-amino-3-phosphonopropionic acid, 3-hydroxybutyrate, 5-hydroxyindoleacetic acid, 5-hydroxytryptophol glucuronide, 7-methylguanosine, acetyldigitoxin, alpha-CEHC, D-glucose, formamidopyrimidine nucleoside triphosphate, gamma glutamylglutamic acid, gamma-glutamylthreonine, ganglioside GA1 (d18:1/16:0), ganglioside GM2 (d18:1/18:1(11z)), L-beta-aspartyl-L-threonine, leucyl-valine, leukotriene F4, L-isoleucine, L-leucine, lysoPC(22:2(13z,16z)), N-a-acetyl-L-arginine, N-acetyltryptophan, N-gamma-L-glutamyl-D-alanine, PG(18:3(9Z,12Z,15Z)/20:3(5Z,8Z,11Z)), phenylalanylphenylalanine, phosphoribosyl-AMP, p-hydroxyphenylacetic acid, PS(14:0/14:1(9Z)), PS(20:4(5Z,8Z,11Z,14Z)/14:1(9Z)), pyruvate, tetrahexosylceramide (d18:1/16:0), trans-3-hydroxycotinine glucuronide, trimethylamine N-oxide, uridine, valine, vanylglycol, L-carnitine, tiglyl-CoA, 2,2-dimethylsuccinic acid, 3-methyl-2-oxovalerate, anserine, L-phenylalanine, L-tryptophan, N,N-Dimethylglycine, N6-acetyllysine, N-acetylysteine, propylene glycol, undecanoic acid, and xanthurenate.

Embodiment 95. The method of embodiment 92 or embodiment 93, wherein said altered level of said at least five metabolites is a decreased level, and wherein said at least five metabolites are selected from the group consisting of 1-methylhistidine, 2-aminomuconic acid semialdehyde, 2-methylguanosine, 2-oxoarginine, 2-pyrroloylglycine, 3-(3,5-diiodo-4-hydroxyphenyl)pyruvate, 3-dehydroxycarnitine, 3-hydroxyoctanoyl carnitine, 3-nitrotyrosine, 3′-O-methyladenosine, 4-guanidinobutanoic acid, 7-methylguanine, alanyl-serine, allantoic acid, argininic acid, arginyl-methionine, citric acid, creatine, cysteic acid, cysteinyl-cysteine, DL-2-aminooctanoic acid, dUMP, gamma-glutamyltyrosine, glutaminyl-lysine, glutarylcarnitine, glycerophosphocholine, glyceryl lactopalmitate, hippuric acid, IDP, L-acetylcarnitine, L-alanine, L-aspartyl-4-phosphate, L-carnitine, L-glutamine, L-histidine, L-isoleucine, L-phenylalanine, L-tyrosine, melatonin, N1-methyl-2-pyridone-5-carboxamide, N6,N6,N6-trimethyl-L-lysine, N-acetyl-L-methionine, nicotine imine, N-ribosylhistidine, O-isobutyryl-L-carnitine, phenol sulphate, phenylpyruvic acid, picolinoylglycine, proline betaine, pyrogallol-2-O-glucuronide, riboflavin, serinyl-hydroxyproline, sphinganine 1-phosphate, trans-3-coumarate, tyrosyl-glutamate, urocanic acid, 1H-indole-3-carboxaldehyde, 2-hydroxyadenine, allodesmosine, arginyl-histidine, guanine, hypoxanthine, LysoPC(14:0), LysoPC(15:0), LysoPC(18:3(6Z,9Z,12Z)), LysoPE(0:0/18:1(11Z)), phosphoric acid, pyrrolidine, uridine, 1,9-dimethylurate, 2-(3-carboxy-3-aminopropyl)-L-histidine, adenosine diphosphate ribose, adenosine thiamine triphosphate, ADP, ATP, cholesterol glucuronide, D-galactose, D-glucose, GTP, malonyl-camitin, N-acetyl-D-glucosamine, NAD⁺, NADH, NADP⁺, N-decanoylglycine, phosphoribosyl-AMP, purine, and tiglylcarnitine.

Embodiment 96. The method of any one of embodiments 92-95, wherein said detecting comprises LC-MS, NMR, GC-MS, CE-MS, FTIR, or combinations thereof.

Embodiment 97. A method for monitoring a mammal having been administered a radiation counter measure following radiation exposure, wherein said method comprises:
(a) detecting a presence or absence of at least one enriched metabolic pathway in a sample from said mammal;
(b) classifying said radiation counter measure as being an effective treatment for said mammal if said presence of said enriched metabolic pathways is not detected; and
(c) administering said radiation counter measure to said mammal.

Embodiment 98. A method for monitoring a mammal having been administered a first radiation counter measure following radiation exposure, wherein said method comprises:
(a) detecting a presence or absence of at least one enriched metabolic pathway in a sample from said mammal;
(b) classifying said first radiation counter measure as not being an effective treatment for said mammal if said presence of said enriched metabolic pathways is detected; and
(c) administering a second radiation counter measure to said mammal.

Embodiment 99. The method of embodiment 97 or embodiment 98, wherein said at least one enriched metabolic pathway is selected from the group consisting of Warburg effect, arginine and proline metabolism, glutamate metabolism, pyruvate metabolism, fatty acid metabolism, propanoate metabolism, nicotinate and nicotinamide metabolism, gluconeogenesis, ammonia recycling, pyrimidine metabolism, urea cycle, selenoamino acid metabolism, mitochondrial beta-oxidation of long chain saturated fatty acids, mitochondrial beta-oxidation of short chain saturated fatty acids, cysteine metabolism, oxidation of branched chain fatty acids, glycolysis, purine metabolism, carnitine synthesis, glutathione metabolism, lactose synthesis, beta oxidation of very long chain fatty acids, and valine, leucine and isoleucine degradation.

Embodiment 100. The method of any one of embodiments 97-99, wherein said mammal is a human.

Embodiment 101. The method of any one of embodiments 97-100, wherein said sample is selected from the group consisting of whole blood, serum, plasma, urine, CSF, saliva, jejunum tissue, lung tissue, heart tissue, kidney tissue, skin tissue, bone marrow, gastrointestinal tract tissue, cardiovascular system tissue, CNS tissue, hematopoietic cells, and a fecal sample.

Embodiment 102. The method of any one of embodiments 97-101, wherein said radiation exposure occurs during an event selected from the group consisting of an industrial accident, terrorist attack, military action, commercial exposure, medical procedure, environmental exposure, and any combinations thereof.

Embodiment 103. The method of any one of embodiments 97-102, wherein said radiation exposure comprises a radiation source selected from the group consisting of ionizing radiation, non-ionizing radiation, low energy particles, and high energy particles.

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1-13. (canceled)

14. A method for treating a mammal having cancer, wherein said method comprises:
(a) detecting an absence of an altered level of at least five metabolites in a sample from said mammal; and
(b) administering a radiation therapy to said mammal.

15. The method of claim 14, wherein said altered level of said at least five metabolites is an increased level, and wherein said at least five metabolites are selected from the group consisting of 10,11-dihydro-12r-hydroxy-leukotriene E4, 12-oxo-20-trihydroxy-leukotriene B4, 2-amino-3-phosphonopropionic acid, 3-hydroxybutyrate, 5-hydroxyindoleacetic acid, 5-hydroxytryptophol glucuronide, 7-methylguanosine, acetyldigitoxin, alpha-CEHC, D-glucose, formamidopyrimidine nucleoside triphosphate, gamma glutamylglutamic acid, gamma-glutamylthreonine, ganglioside GA1 (d18:1/16:0), ganglioside GM2 (d18:1/18:1(11z)), L-beta-aspartyl-L-threonine, leucyl-valine, leukotriene F4, L-isoleucine, L-leucine, lysoPC(22:2(13z,16z)), N-a-acetyl-L-arginine, N-acetyltryptophan, N-gamma-L-glutamyl-D-alanine, PG(18:3(9Z,12Z,15Z)/20:3(5Z,8Z,11Z)), phenylalanylphenylalanine, phosphoribosyl-AMP, p-hydroxyphenylacetic acid, PS(14:0/14:l(9Z)), PS(20:4(5Z,8Z,11Z,14Z)/14:1(9Z)), pyruvate, tetrahexosylceramide (d18:1/16:0), trans-3-hydroxycotinine glucuronide, trimethylamine N-oxide, uridine, valine, vanylglycol, L-carnitine, tiglyl-CoA, 2,2-dimethylsuccinic acid, 3-methyl-2-oxovalerate, anserine, L-phenylalanine, L-tryptophan, N,N-Dimethylglycine, N6-acetyllysine, N-acetylysteine, propylene glycol, undecanoic acid, and xanthurenate.

16. The method of claim 14, wherein said altered level of said at least five metabolites is a decreased level, and wherein said at least five metabolites are selected from the group consisting of 1-methylhistidine, 2-aminomuconic acid semialdehyde, 2-methylguanosine, 2-oxoarginine, 2-pyrroloylglycine, 3-(3,5-diiodo-4-hydroxyphenyl)pyruvate, 3-dehydroxycarnitine, 3-hydroxyoctanoyl carnitine, 3-nitrotyrosine, 3′-O-methyladenosine, 4-guanidinobutanoic acid, 7-methylguanine, alanyl-serine, allantoic acid, argininic acid, arginyl-methionine, citric acid, creatine, cysteic acid, cysteinyl-cysteine, DL-2-aminooctanoic acid, dUMP, gamma-glutamyltyrosine, glutaminyl-lysine, glutarylcarnitine, glycerophosphocholine, glyceryl lactopalmitate, hippuric acid, IDP, L-acetylcarnitine, L-alanine, L-aspartyl-4-phosphate, L-carnitine, L-glutamine, L-histidine, L-isoleucine, L-phenylalanine, L-tyrosine, melatonin, N1-methyl-2-pyridone-5-carboxamide, N6,N6,N6-trimethyl-L-lysine, N-acetyl-L-methionine, nicotine imine, N-ribosylhistidine, 0-isobutyryl-L-carnitine, phenol sulphate, phenylpyruvic acid, picolinoylglycine, proline betaine, pyrogallol-2-O-glucuronide, riboflavin, serinyl-hydroxyproline, sphinganine 1-phosphate, trans-3-coumarate, tyrosyl-glutamate, urocanic acid, 1H-indole-3-carboxaldehyde, 2-hydroxyadenine, allodesmosine, arginyl-histidine, guanine, hypoxanthine, LysoPC(14:0), LysoPC(15:0), LysoPC(18:3(6Z,9Z,12Z)), LysoPE(0:0/18:1(11Z)), phosphoric acid, pyrrolidine, uridine, 1,9-dimethylurate, 2-(3-carboxy-3-aminopropyl)-L-histidine, adenosine diphosphate ribose, adenosine thiamine triphosphate, ADP, ATP, cholesterol glucuronide, D-galactose, D-glucose, GTP, malonyl-carnitin, N-acetyl-D-glucosamine, NAD⁺, NADH, NADP⁺, N-decanoylglycine, phosphoribosyl-AMP, purine, and tiglylcarnitine.

17. The method of claim 14, wherein said detecting comprises LC-MS, NMR, GC-MS, CE-MS, FTIR, or combinations thereof.

18. A method for treating a mammal having cancer, wherein said method comprises:
(a) detecting an absence of at least one enriched metabolic pathway in a sample from said mammal; and
(b) administering a radiation therapy to said mammal.

19. The method of claim 18, wherein said at least one enriched metabolic pathway is selected from the group consisting of Warburg effect, arginine and proline metabolism, glutamate metabolism, pyruvate metabolism, fatty acid metabolism, propanoate metabolism, nicotinate and nicotinamide metabolism, gluconeogenesis, ammonia recycling, pyrimidine metabolism, urea cycle, selenoamino acid metabolism, mitochondrial beta-oxidation of long chain saturated fatty acids, mitochondrial beta-oxidation of short chain saturated fatty acids, cysteine metabolism, oxidation of branched chain fatty acids, glycolysis, purine metabolism, carnitine synthesis, glutathione metabolism, lactose synthesis, beta oxidation of very long chain fatty acids, and valine, leucine and isoleucine degradation.

20. The method of claim 14, wherein said mammal is a human.

21. The method of claim 14, wherein said sample is selected from the group consisting of whole blood, serum, plasma, urine, CSF, saliva, jejunum tissue, lung tissue, heart tissue, kidney tissue, skin tissue, bone marrow, gastrointestinal tract tissue, cardiovascular system tissue, CNS tissue, hematopoietic cells, and a fecal sample.

22. A method for treating a mammal having cancer, wherein said method comprises:
(a) detecting a presence of an altered level of at least five metabolites in a sample from said mammal; and
(b) administering a cancer treatment to said mammal, wherein said cancer treatment is not a radiation therapy.

23. The method of claim 22, wherein said altered level of said at least five metabolites is an increased level, and wherein said at least five metabolites are selected from the group consisting of 10,11-dihydro-12r-hydroxy-leukotriene E4, 12-oxo-20-trihydroxy-leukotriene B4, 2-amino-3-phosphonopropionic acid, 3-hydroxybutyrate, 5-hydroxyindoleacetic acid, 5-hydroxytryptophol glucuronide, 7-methylguanosine, acetyldigitoxin, alpha-CEHC, D-glucose, formamidopyrimidine nucleoside triphosphate, gamma glutamylglutamic acid, gamma-glutamylthreonine, ganglioside GA1 (d18:1/16:0), ganglioside GM2 (d18:1/18:1(11z)), L-beta-aspartyl-L-threonine, leucyl-valine, leukotriene F4, L-isoleucine, L-leucine, lysoPC(22:2(13z,16z)), N-a-acetyl-L-arginine, N-acetyltryptophan, N-gamma-L-glutamyl-D-alanine, PG(18:3(9Z,12Z,15Z)/20:3(5Z,8Z,11Z)), phenylalanylphenylalanine, phosphoribosyl-AMP, p-hydroxyphenylacetic acid, PS(14:0/14:1(9Z)), PS(20:4(5Z,8Z,11Z,14Z)/14:1(9Z)), pyruvate, tetrahexosylceramide (d18:1/16:0), trans-3-hydroxycotinine glucuronide, trimethylamine N-oxide, uridine, valine, vanylglycol, L-carnitine, tiglyl-CoA, 2,2-dimethylsuccinic acid, 3-methyl-2-oxovalerate, anserine, L-phenylalanine, L-tryptophan, N,N-Dimethylglycine, N6-acetyllysine, N-acetylysteine, propylene glycol, undecanoic acid, and xanthurenate.

24. The method of claim 22, wherein said altered level of said at least five metabolites is a decreased level, and wherein said at least five metabolites are selected from the group consisting of 1-methylhistidine, 2-aminomuconic acid semialdehyde, 2-methylguanosine, 2-oxoarginine, 2-pyrroloylglycine, 3-(3,5-diiodo-4-hydroxyphenyl)pyruvate, 3-dehydroxycarnitine, 3-hydroxyoctanoyl carnitine, 3-nitrotyrosine, 3′-O-methyladenosine, 4-guanidinobutanoic acid, 7-methylguanine, alanyl-serine, allantoic acid, argininic acid, arginyl-methionine, citric acid, creatine, cysteic acid, cysteinyl-cysteine, DL-2-aminooctanoic acid, dUMP, gamma-glutamyltyrosine, glutaminyl-lysine, glutarylcarnitine, glycerophosphocholine, glyceryl lactopalmitate, hippuric acid, IDP, L-acetylcarnitine, L-alanine, L-aspartyl-4-phosphate, L-carnitine, L-glutamine, L-histidine, L-isoleucine, L-phenylalanine, L-tyrosine, melatonin, N1-methyl-2-pyridone-5-carboxamide, N6,N6,N6-trimethyl-L-lysine, N-acetyl-L-methionine, nicotine imine, N-ribosylhistidine, 0-isobutyryl-L-carnitine, phenol sulphate, phenylpyruvic acid, picolinoylglycine, proline betaine, pyrogallol-2-O-glucuronide, riboflavin, serinyl-hydroxyproline, sphinganine 1-phosphate, trans-3-coumarate, tyrosyl-glutamate, urocanic acid, 1H-indole-3-carboxaldehyde, 2-hydroxyadenine, allodesmosine, arginyl-histidine, guanine, hypoxanthine, LysoPC(14:0), LysoPC(15:0), LysoPC(18:3(6Z,9Z,12Z)), LysoPE(0:0/18:1(11Z)), phosphoric acid, pyrrolidine, uridine, 1,9-dimethylurate, 2-(3-carboxy-3-aminopropyl)-L-histidine, adenosine diphosphate ribose, adenosine thiamine triphosphate, ADP, ATP, cholesterol glucuronide, D-galactose, D-glucose, GTP, malonyl-carnitin, N-acetyl-D-glucosamine, NAD⁺, NADH, NADP⁺, N-decanoylglycine, phosphoribosyl-AMP, purine, and tiglylcarnitine.

25. The method of claim 22, wherein said detecting comprises LC-MS, NMR, GC-MS, CE-MS, FTIR, or combinations thereof.

26. A method for treating a mammal having cancer, wherein said method comprises:
(a) detecting a presence of at least one enriched metabolic pathway in a sample from said mammal; and
(b) administering a cancer treatment to said mammal, wherein said cancer treatment is not a radiation therapy.

27. The method of claim 26, wherein said at least one enriched metabolic pathway is selected from the group consisting of Warburg effect, arginine and proline metabolism, glutamate metabolism, pyruvate metabolism, fatty acid metabolism, propanoate metabolism, nicotinate and nicotinamide metabolism, gluconeogenesis, ammonia recycling, pyrimidine metabolism, urea cycle, selenoamino acid metabolism, mitochondrial beta-oxidation of long chain saturated fatty acids, mitochondrial beta-oxidation of short chain saturated fatty acids, cysteine metabolism, oxidation of branched chain fatty acids, glycolysis, purine metabolism, carnitine synthesis, glutathione metabolism, lactose synthesis, beta oxidation of very long chain fatty acids, and valine, leucine and isoleucine degradation.

28. The method of claim 22, wherein said mammal is a human.

29. The method of claim 22, wherein said sample is selected from the group consisting of whole blood, serum, plasma, urine, CSF, saliva, jejunum tissue, lung tissue, heart tissue, kidney tissue, skin tissue, bone marrow, gastrointestinal tract tissue, cardiovascular system tissue, CNS tissue, hematopoietic cells, and a fecal sample.

30. The method of claim 22, wherein said cancer treatment comprises administering an anti-cancer agent selected from the group consisting of a chemotherapeutic agent, a targeted cancer drug, an immunotherapy drug, and a hormone therapy drug.

31. The method of claim 22, wherein said cancer treatment comprises surgery.

32-45. (canceled)

46. The method of claim 18, wherein said mammal is a human.

47. The method of claim 18, wherein said sample is selected from the group consisting of whole blood, serum, plasma, urine, CSF, saliva, jejunum tissue, lung tissue, heart tissue, kidney tissue, skin tissue, bone marrow, gastrointestinal tract tissue, cardiovascular system tissue, CNS tissue, hematopoietic cells, and a fecal sample.

48. The method of claim 26, wherein said mammal is a human.

49. The method of claim 26, wherein said sample is selected from the group consisting of whole blood, serum, plasma, urine, CSF, saliva, jejunum tissue, lung tissue, heart tissue, kidney tissue, skin tissue, bone marrow, gastrointestinal tract tissue, cardiovascular system tissue, CNS tissue, hematopoietic cells, and a fecal sample.

50. The method of claim 26, wherein said cancer treatment comprises administering an anti-cancer agent selected from the group consisting of a chemotherapeutic agent, a targeted cancer drug, an immunotherapy drug, and a hormone therapy drug.

51. The method of claim 26, wherein said cancer treatment comprises surgery.
